SEQUENCE LISTING (171 sequences)

SEQ ID NO: 1 - DNA, 4572 bp, Artificial Sequence
  Synthetic polynucleotide
tcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagggcggccgcggcgcgccgttccggatctgcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagcttgacattgtaggacgtttaaacattaccctgttatccctaggccggcctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgttaattaagcggccgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccg

SEQ ID NO: 2 - DNA, 2474 bp, Artificial Sequence
  Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagggcggccgccagctggaattctacgtactgcagagtactgcggccgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 6638 bp, Artificial Sequence
  Synthetic polynucleotide
tcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagggcggccgcggcgcgccgttccggatctgcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagcttgacattgtaggacgtttaaacattaccctgttatccctaggatcctacgtatacatactccgaaggaggacaaatttatttattgtggtacaataaataagtggtacaataaataaattgtatgtaaaccccttccccttcgggacgtccccttacgggaatataaatattagtggcagttgcctgccaacaaatttatttattgtattaacataggcagtggcggtaccactgccactggcgtcctaatataaatattgggcaactaaagtttatcgcagtattaacataggcagtggcggtaccactgccactggcgtcctccttcggagtatgtaaacctgctaccgcagcaaataaattttattctattttaatactacaatatttagattcccgttaggggataggccaggcaattgtcactggcgtcatagtatatcaatattgtaacagattgacaccctttaagtaaacattttttttaggattcatatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcaatgacaattgcgatcggtacatatcaagagaaacgcacatggttcgatgacgctgatgactggcttcgtcaagaccgtttcgtattcgtaggttggtcaggtttattactattcccttgtgcttactttgcaactccggtccggcggccgcctcgagacgacttgtccgcttcatcagacacggctttcctaaccatcaatggtggattttcaggaaagacgtttaaagaagtggcataaagtttatttgttgaagaattggttttgtttccattcaaagaattgttagggataaaactttgcatttttttataatttgttataagtttttcaaacttatatgtttttaaaaatgcatttaattgcttattaatgcgttcattttgtaatgtttcaataggtcttgcttgcgctaatcgcagtattcccgatactttgtctgcttgtttttcgggtattgagaataagtaagtataatgatttaaaaaagtcatgttttgattaaatcttttttatatggttaaaaacattatggtatatctaaataaatttattttttactaaatctccaatttgcaatttagagatataattaaaactataaagttatttaagttaatttgtaatcaaatccaacacaaaaatgtttttatatagttaacatgttaaatttaacatatgttaaacaactaaaattctgtaacagagaacaataaaataaatgctagattttgtgtaatgccgaagtatatttatatacttccctttcaaaaaaataaatactcttgccactaaaattcatttgcctaggacgtccccttccccttacgggatgtttatatactaggacgtccccttccccttacgggatatttatatactccgaaggacgtccccttcgggcaaataaattttagtggcagttgcctgccaactgcctaggcaagtaaacttagggattttaatgcaataaataaatttgtccccttacgggacgtcagtggcagttgcctgccaactgcctaatataaatattagtggatatttatatactccgaaggaggcagttacctgccaactgccgaggcaaataaattttagtggcagtggtaccgccactgcctgctccctccttccccttcgggcaagtaaacttagcatgttgtcgacattaccctgttatccctaggccggcctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgttaattaagcggccgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccg

SEQ ID NO: 4 - DNA, 7855 bp, Artificial Sequence
  Synthetic polynucleotide
gtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgcggccgcagtactctgcagattttatgcaaaattaaagtcttgtgacaacagctttctccttaagtgcaaatatcgcccattctttcctcttttcgtatataaatgctgtaatagtaggatgtcgtacccgtaaaggtacgacattgaatattaatatactcctaagtttactttcccaatatttatattaggacgtccccttcgggtaaataaattttagtggcagtggtaccgccactccctattttaatactgcgaaggaggcagttggcaggcaactcgtcgttcgcagtatataaatatccactaatatttatattcccgtaaggggacgtcccgaaggggaaggggaaagaagcagtcgcctccttgcgaaaaggtttacttgcccgaccagtgaaaagcatgctgtaagatataaatctaccctgaaagggatgcatttcaccataatactatacaaatggtgttaccctttgaggatcataacggtgctactggaatatatggtctcttcatggatagacgatagccatttatttacccattaaggggacattagtggcctgtcactgctccttacgagacgccagtggacgttcgtcctagaaaatttatgcgctgcctagaagccccaaaagggaagtttactgactcgttagagcgtgcgctaacaggtttaaatacttcaatatgtatattaggacgccggtggcagtggtaccgccactgccaccgtcggaggacgtcccttacggtatattatatactaggattttaatactccgaaggaggcagtggcggtaccactgccactaatatttatattcccgtaagggacgtcctccttcggagtatgtaaacattctaagtttacttgcccaatatttatattaggcagttggcaggcaactgctagctctcctccttcggagtatgtaaacatcgcagtatataaatatccactaatatttatattcccgtaaggggacgtcccgaaggggaaggggaaggacgtcagtggcagttgcctgccaactgcctaggcaagtaaacttaggagtatataaatataggcagtcgcggtaccactgccactgacgtcctgccaactgcctaggcaagtaaacttaagtggcactaaaatgcatttgcccgaaggggaaggaggacgccagtggcagtggtaccgccactgcctccttcggagtattaaaatcctagtatgtaaatctgctagcgcaggaaataaattttattctatttatatactccgttaggaggtaagtaaaccccttccccttcgggacgtcagtgcagttgcctgccaactgcctaatataaatattagaccactaaagtttggcaactgccaactgttgtccttcggaggaaaaaaaatggttaactcgcaagcagttaacataactaaagtttgttactttaccgaagacgtttaccctttctcggttaaggagacggagacagttgcactgtgactgcctagtatagcaattttgtttttgtttatatgctcgacaaaatgactttcataaaaatataaagtagttagctagttattttatatcactataactagggttctcagaggcaccgaagtcacttgtaaaaatagtactttttaacttgtttaatcttcgtgttcttcaaaaggatcacgtaatttttttgaaggtggaccaaaactaacataaactgaatagccagttacacttaacagaagaaaccataaaaaaaaggtaaagaaaaaagctggactttccatagctcatttaataataaaattattctcttttcaacatatctcttagatagttcaaaagacttgacgactgtgtcccacatttttaaacaaaattaatctactcaaaattttgccctgagaaagaataacttacttcgtttttgcagtagccattcatgtcactttgaaactgtccttacaaagttaaacattaattaaaaattatttaatttttatataacaaatattatattaaataaaaaatgaacaaagaacttctaagatcgtctttagtgagtaattaaagagttttacttaccagacaaggcagttttttcattcttttaaagcaggcagttctgaaggggaaaagggactgcctactgcggtcctaggtaaatacatttttatgcaatttatttcttgtgctagtaggtttctatactcacaagaagcaaccccttgacgagagaacgttatcctcagagtatttataatcctgagagggaatgcactgaagaatattttccttattttttacagaaagtaaataaaatagcgctaataacgcttaattcatttaatcaattatggcaacaggaacttctaaagctaaaccatcaaaagtaaattcagacttccaagaacctggtttagttacaccattaggtactttattacgtccacttaactcagaagcaggtaaagtattaccaggctggggtacaactgttttaatggctgtatttatccttttatttgcagcattcttattaatcattttagaaatttacaacagttctttaattttagatgacgtttctatgagttgggaaactttagctaaagtttcttaattttatttaacacaaacataaaatataaaactgtttgttaaggctagctgctaagtcttcttttcgctaaggtaaactaagcaactcaaccatatttatattcggcagtggcaccgccaactgccactggccttccgttaagataaacgcgtggatctcacgtgactagtgatatctacgtaatcgatgaattcgatcccatttttataactggatctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcgtatggaattgcccaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaatttatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagaccttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattgattccaagcattatctaaaatactctgcaggcacgctagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttatttttatggtgtttaggttaaatacctaaacatcattttacatttttaaaattaagttctaaagttatcttttgtttaaatttgcctgtctttataaattacgatgtgccagaaaaataaaatcttagctttttattatagaatttatctttatgtattatattttataagttataataaaagaaatagtaacatactaaagcggatgtagcgcgtttatcttaacggaaggaattcggcgcctacgtacccgggtcgcgaggatccacgcgttaatagctcacttttctttaaatttaatttttaatttaaaggtgtaagcaaattgcctgacgagagatccacttaaaggatgacagtggcgggctactgcctacttccctccgggataaaatttatttgaaaaacgttagttacttcctaacggagcattgacatccccatatttatattaggacgtccccttcgggtaaataaattttagtggacgtccccttcgggcaaataaattttagtggacaataaataaatttgttgcctgccaactgcctaggcaagtaaacttgggagtattaaaataggacgtcagtggcagttgcctgccaactgcctatatttatatactgcgaagcaggcagtggcggtaccactgccactggcgtcctaatataaatattgggcaactaaagtttatagcagtattaacatcctatatttatatactccgaaggaacttgttagccgataggcgaggcaacaaatttatttattgtcccgtaaaaggatgcctccagcatcgaaggggaaggggacgtcctaggccataaaactaaagggaaatccatagtaactgatgttataaatttatagactccaaaaaacagctgcgttataaataacttctgttaaatatggccaaggggacaggggcactttcaactaagtgtacattaaaaattgacaattcaattttttttaattataatatatatttagtaaaatataacaaaaagcccccatcgtctaggtagaattccagctggcggccgccctatg

SEQ ID NO: 5 - DNA, 8458 bp, Artificial Sequence
  Synthetic polynucleotide
tcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagggcggccgcggcgcgccgttccggatctgcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagcttgacattgtaggacgtttaaacattaccctgttatccctaggatcctacgtaatcgatgaattcgatcccatttttataactggatctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcgtatggaattgcccaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaatttatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagaccttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattgattccaagcattatctaaaatactctgcaggcacgctagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttatttttatggtgtttaggttaaatacctaaacatcattttacatttttaaaattaagttctaaagttatcttttgtttaaatttgcctgtctttataaattacgatgtgccagaaaaataaaatcttagctttttattatagaatttatctttatgtattatattttataagttataataaaagaaatagtaacatactaaagcggatgtagcgcgtttatcttaacggaaggaattcggcgcctacgtatacatactccgaaggaggacaaatttatttattgtggtacaataaataagtggtacaataaataaattgtatgtaaaccccttccccttcgggacgtccccttacgggaatataaatattagtggcagttgcctgccaacaaatttatttattgtattaacataggcagtggcggtaccactgccactggcgtcctaatataaatattgggcaactaaagtttatcgcagtattaacataggcagtggcggtaccactgccactggcgtcctccttcggagtatgtaaacctgctaccgcagcaaataaattttattctattttaatactacaatatttagattcccgttaggggataggccaggcaattgtcactggcgtcatagtatatcaatattgtaacagattgacaccctttaagtaaacattttttttaggattcatatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcaatgacaattgcgatcggtacatatcaagagaaacgcacatggttcgatgacgctgatgactggcttcgtcaagaccgtttcgtattcgtaggttggtcaggtttattactattcccttgtgcttactttgcaactccggtccggcggccgcctcgagacgacttgtccgcttcatcagacacggctttcctaaccatcaatggtggattttcaggaaagacgtttaaagaagtggcataaagtttatttgttgaagaattggttttgtttccattcaaagaattgttagggataaaactttgcatttttttataatttgttataagtttttcaaacttatatgtttttaaaaatgcatttaattgcttattaatgcgttcattttgtaatgtttcaataggtcttgcttgcgctaatcgcagtattcccgatactttgtctgcttgtttttcgggtattgagaataagtaagtataatgatttaaaaaagtcatgttttgattaaatcttttttatatggttaaaaacattatggtatatctaaataaatttattttttactaaatctccaatttgcaatttagagatataattaaaactataaagttatttaagttaatttgtaatcaaatccaacacaaaaatgtttttatatagttaacatgttaaatttaacatatgttaaacaactaaaattctgtaacagagaacaataaaataaatgctagattttgtgtaatgccgaagtatatttatatacttccctttcaaaaaaataaatactcttgccactaaaattcatttgcctaggacgtccccttccccttacgggatgtttatatactaggacgtccccttccccttacgggatatttatatactccgaaggacgtccccttcgggcaaataaattttagtggcagttgcctgccaactgcctaggcaagtaaacttagggattttaatgcaataaataaatttgtccccttacgggacgtcagtggcagttgcctgccaactgcctaatataaatattagtggatatttatatactccgaaggaggcagttacctgccaactgccgaggcaaataaattttagtggcagtggtaccgccactgcctgctccctccttccccttcgggcaagtaaacttagcatgttgtcgacattaccctgttatccctaggccggcctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgttaattaagcggccgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccg

SEQ ID NO: 6 - DNA, 22908 bp, Artificial Sequence
  Synthetic polynucleotide
tgaatttatcagatctaactgaggagtaagaaacccccatgtcaaagaaaaacagaccaacaattgggcgaacccttaatccttcaatattaagcggatttgatagttcttcagcctctggcgatcgagtcgagcaggtattcaagttatcaactggtcgccaggccacatttattgaagaggtaatacctccgaaccaggtagaaagcgatacctttgttgatcagcataacaacgggcgtgaccaggcatctcttacgccaaaatcattaaaaagtatccgaagcactattaagcatcagcaattttaccctgcaataggtgttagacgggctacagggaaaattgaaattttggatggttcccggcgtcgagcttctgccatcttagagaacgtagggttgcgggttttagtcacggaccaggagatcagcgttcaggaagcgcaaaatttagcgaaagacgttcagacagcattgcagcacagcattcgagaaataggtctgcgtttgatgcgaatgaaaaatgatgggatgagtcagaaggatattgcagccaaagaagggctgtctcaggcgaaggtcacgcgtgctctccaggcagcgagtgctccggaagaattagtcgcccttttccctgtgcagtcggaattaaccttttcggactacaaaacgctttgtgctgttggcgacgaaatggggaacaagaatttagagtttgatcagcttattcaaaacatatccccggaaataaacgacatcttatccattgaagaaatggccgaagatgaagttaaaaataaaatcctgcgcttgataacaaaggaagcctcactactcacggataaaggttctaaagataagtccgtagttactgaattatggaaatttgaggacaaggatcgctttgcaaggaagcgcgtgaaaggccgtgcattttcttatgagtttaatcgactctcaaaagagttacaggaagaactcgacaggatgattgggcatatccttagaaagagcctcgataaaaagccgaagccttaaactttcgccattcaaatttcactattaactgactgtttttaaagtaaattactctaaaatttcaaggtgaaatcgccacgatttcaccttggattttaccttcctcccctcctcccgaaaaaaataaaaaaattgcttgtcacgagaaagtcaacaagtgactttcaataaaatctcttccgaaaagggattcacacaagtgccttgtgtttaaggaagagtaaattgagtaacttacgcgaataccagaatcgtattgcagatatcgcaaaacgctctaaagctgtgcttggctgggcaagcactgcgcagttcggtactgataaccaattcattaaagatgatgccgcgcgtgccgcatctatccttgaagctgcacgtaaagacccggtttttgcgggtatctctgataatgccaccgctcaaatcgctacagcgtgggcaagtgcactggctgactacgccgcagcacataaatctatgccgcgtccggaaattctggcctcctgccaccagacgctggaaaactgcctgatagagtccacccgcaatagcatggatgccactaataaagcgatgctggaatctgtcgcagcagagatgatgagcgtttctgacggtgttatgcgtctgcctttattcctcgcgatgatcctgcctgttcagttgggggcagctaccgctgatgcgtgtaccttcattccggttacgcgtgaccagtccgacatctatgaagtctttaacgtggcaggttcatcttttggttcttatgctgctggtgatgttctggacatgcaatccgtcggtgtgtacagccagttacgtcgccgctatgtgctggtggcaagctccgatggcaccagcaaaaccgcaaccttcaagatggaagacttcgaaggccagaatgtaccaatccgaaaaggtcgcactaacatctacgttaaccgtattaagtctgttgttgataacggttccggcagcctacttcactcgtttactaatgctgctggtgagcaaatcactgttacctgctctctgaactacaacattggtcagattgccctgtcgttctccaaagcgccggataaaggcactgagatcgcaattgagacggaaatcaatattgaagccgctcctgagctgatcccgctgatcaaccacgaaatgaagaaatacaccctgttcccaagtcagttcgttatcgcggctgagcacacggtacaggcggcgtatgaagcacagcgtgaatttggtctggacctgggttccctacagttccgcaccctgaaggaatacctgtctcatgaacaggatatgctgcgtcttcgcatcatgatctggcgcactcttgcgaccgacacctttgacatcgctctgccggttaaccagtcctttgatgtatgggcaaccatcattcgtggcaaattccagactgtatatcgcgacattattgagcgcgttaaatcttctggtgcgatggggatgtttgctggtgctgatgcagcatctttcttcaaacagttgccgaaggatttcttccagccagccgaagactatatccagactccgtatgttcactacatcggtaccccatttaggaccacccacagcacctaacaaaacggcatcagccttcttggaggcttccagcgcctcatctggaagtggaacacctgtagcatcgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtagatatactagaagttctcctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctacaactccacttattgttaggtagaattgtccgttagttgtttattaattgcaataatggggcgtccagttttggcaacagtgtcctcttaccaggacacctatgagtttgcctcatggcaaactagaggtgttgaaagtatgcatggttataattagagcaattcattaccctctgaatcctgccggtataccccattgttcgttatctttatttttggctaaaaccgcattaagagcttcgtttaccgtcatgcaatgcggtaggttatcgaagtttgatatcccgccaatatcaggcgaacgcttgttcttcaggtaagcatatttccgcgcagccgcctctactttctgcttgaactcatgtttttgagtgcgttttttggataaccgcagattgtcagcctttgcttttgccttagcgatccatgaagtcaattttttgaggctggttgttccggcaccgccggaaactgatctttttgtttttttaacttgtgacttcttattctttattgccacgtcatcctgacagggggagggggtatcattttgacatgggggtgtggataaaaaattaaataaagccaatgtcttagcgagaacagctttaaccttggttgccgctgaagagatctttaatttgctttctatcagcgcatttttggcttgttgtgcgaaggccaaaaaggatggtgtaaaccggtacaggttagcgcgacgttcacggtgatcgccgataacaatctctacagacagaattcctttgtttacagcttcacggaatgcacgaacgacggttgattggctataaccagtttctgccgcgatcaggcggtgaggcttgtgaatgaagtattcactggttgttgccgcgagatttgcacattgcgacaggatatgcccggcgctacgggatagaccggagtgtgttacaaagcaggccaattcatagccagaaaaagtaaaatcgctcatcgttatacagctcaggaaagtgactttagccagcattacaatgctggtggttcttactacgtctgttagcgcgttgccgcgacaggtaccagcacaccagcatcaagcaatcgcttcatcagccactgctgacctttgccggttatacgagtcgtgaaagaaatcctgcttccattgcttgtatcgatcacggtttctttaagggtgaaatacccacgagatatgtattcttgtttggggacgttcctgcgttcaccggttgcgatcagaattccgttatcacgcaaccaggtgaagagatagttttggcccaggccgagcactttggcatagttgccgattagaaccccgctggcggtagcaacgcgttcagcgaattcgactttaggtgcatccataagcattttttgctccagccgttgcttttgctctgccaggtcggcagccaaacggagagcttcagggagactctgcggaatagcaggttgtaatcttccggttcgatagtcgataaatgtctggtttaccttcagccgaaacgcgggagaaatccagcctgcgtactccacagcgagcaattcatgggcaaaagtgccgccgccacggccttcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggtagtcgatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaggctctcaagggcatcggtcgagcttgacattgtaggacgtttaaacattaccctgttatccctaggatcctacgtaatcgatgaattcgatcccatttttataactggatctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcgtatggaattgcccaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaatttatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagaccttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattgattccaagcattatctaaaatactctgcaggcacgctagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttatttttatggtgtttaggttaaatacctaaacatcattttacatttttaaaattaagttctaaagttatcttttgtttaaatttgcctgtctttataaattacgatgtgccagaaaaataaaatcttagctttttattatagaatttatctttatgtattatattttataagttataataaaagaaatagtaacatactaaagcggatgtagcgcgtttatcttaacggaaggaattcggcgcctacgtatacatactccgaaggaggacaaatttatttattgtggtacaataaataagtggtacaataaataaattgtatgtaaaccccttccccttcgggacgtccccttacgggaatataaatattagtggcagttgcctgccaacaaatttatttattgtattaacataggcagtggcggtaccactgccactggcgtcctaatataaatattgggcaactaaagtttatcgcagtattaacataggcagtggcggtaccactgccactggcgtcctccttcggagtatgtaaacctgctaccgcagcaaataaattttattctattttaatactacaatatttagattcccgttaggggataggccaggcaattgtcactggcgtcatagtatatcaatattgtaacagattgacaccctttaagtaaacattttttttaggattcatatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcaatgacaattgcgatcggtacatatcaagagaaacgcacatggttcgatgacgctgatgactggcttcgtcaagaccgtttcgtattcgtaggttggtcaggtttattactattcccttgtgcttactttgcaactccggtccggcggccgcctcgagacgacttgtccgcttcatcagacacggctttcctaaccatcaatggtggattttcaggaaagacgtttaaagaagtggcataaagtttatttgttgaagaattggttttgtttccattcaaagaattgttagggataaaactttgcatttttttataatttgttataagtttttcaaacttatatgtttttaaaaatgcatttaattgcttattaatgcgttcattttgtaatgtttcaataggtcttgcttgcgctaatcgcagtattcccgatactttgtctgcttgtttttcgggtattgagaataagtaagtataatgatttaaaaaagtcatgttttgattaaatcttttttatatggttaaaaacattatggtatatctaaataaatttattttttactaaatctccaatttgcaatttagagatataattaaaactataaagttatttaagttaatttgtaatcaaatccaacacaaaaatgtttttatatagttaacatgttaaatttaacatatgttaaacaactaaaattctgtaacagagaacaataaaataaatgctagattttgtgtaatgccgaagtatatttatatacttccctttcaaaaaaataaatactcttgccactaaaattcatttgcctaggacgtccccttccccttacgggatgtttatatactaggacgtccccttccccttacgggatatttatatactccgaaggacgtccccttcgggcaaataaattttagtggcagttgcctgccaactgcctaggcaagtaaacttagggattttaatgcaataaataaatttgtccccttacgggacgtcagtggcagttgcctgccaactgcctaatataaatattagtggatatttatatactccgaaggaggcagttacctgccaactgccgaggcaaataaattttagtggcagtggtaccgccactgcctgctccctccttccccttcgggcaagtaaacttagcatgttgtcgacattaccctgttatccctaggccggcctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaatgaaatgagatgatacttgcttatctcatagttaactggcataaattttagtataggttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgatttaggcaaaagttactatttctggctcgtgtaatatatgtatgctaatgtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatattaggacttccacaccaactagtaatatgacaataaattcaagatattcttcatgagaatggcccagctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcaattctttagtataaatttcactctgaaccatcttggaaggaccggataattatttgaaatctctttttcaattgtatatgtgttatgtagtatactctttcttcaacaattaaatactctcggtagccaagttggtttaaggcgcaagactttaatttatcactacggaattggcctattaggcctacccactagtcaattcgggaggatcgaaacggcagatcgcaaaaaacagtacatacagaaggagacatgaacatgaacatcaaaaaaattgtaaaacaagccacagttctgacttttacgactgcacttctggcaggaggagcgactcaagccttcgcgaaagaaaataaccaaaaagcatacaaagaaacgtacggcgtctctcatattacacgccatgatatgctgcagatccctaaacagcagcaaaacgaaaaataccaagtgcctcaattcgatcaatcaacgattaaaaatattgagtctgcaaaaggacttgatgtgtgggacagctggccgctgcaaaacgctgacggaacagtagctgaatacaacggctatcacgttgtgtttgctcttgcgggaagcccgaaagacgctgatgacacatcaatctacatgttttatcaaaaggtcggcgacaactcaatcgacagctggaaaaacgcgggccgtgtctttaaagacagcgataagttcgacgccaacgatccgatcctgaaagatcagacgcaagaatggtccggttctgcaacctttacatctgacggaaaaatccgtttattctacactgactattccggtaaacattacggcaaacaaagcctgacaacagcgcaggtaaatgtgtcaaaatctgatgacacactcaaaatcaacggagtggaagatcacaaaacgatttttgacggagacggaaaaacatatcagaacgttcagcagtttatcgatgaaggcaattatacatccggcgacaaccatacgctgagagaccctcactacgttgaagacaaaggccataaataccttgtattcgaagccaacacgggaacagaaaacggataccaaggcgaagaatctttatttaacaaagcgtactacggcggcggcacgaacttcttccgtaaagaaagccagaagcttcagcagagcgctaaaaaacgcgatgctgagttagcgaacggcgccctcggtatcatagagttaaataatgattacacattgaaaaaagtaatgaagccgctgatcacttcaaacacggtaactgatgaaatcgagcgcgcgaatgttttcaaaatgaacggcaaatggtacttgttcactgattcacgcggttcaaaaatgacgatcgatggtattaactcaaacgatatttacatgcttggttatgtatcaaactctttaaccggcccttacaagccgctgaacaaaacagggcttgtgctgcaaatgggtcttgatccaaacgatgtgacattcacttactctcacttcgcagtgccgcaagccaaaggcaacaatgtggttatcacaagctacatgacaaacagaggcttcttcgaggataaaaaggcaacatttgcgccaagcttcttaatgaacatcaaaggcaataaaacatccgttgtcaaaaacagcatcctggagcaaggacagctgacagtcaactaataacagcaaaaagaaaatgccgatacttcattggcattttcttttatttctcaacaagatggtgaattgactagtgggtagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattgatccggaacccttaatataacttcgtataatgtatgctatacgaagttattaggtccctcgactacgtcgttaaggccgtttctgacagagtaaaattcttgagggaactttcaccattatgggaaatggttcaagaaggtattgacttaaactccatcaaatggtcaggtcattgagtgttttttatttgttgtattttttttttttagagaaaatcctccaatatataaattaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccgcgcttgcggaagcatcagcaaataaggccagcacagccagcgcagttgccgctttggttcctgattctgttcttgatgaattaaacaaagcggcacagtaacaaaggacttcattgataatttttcttcaggaggaagacatgtcattcttttctacgttaaaaacagctttgtctttgaaggagaaacttgctgctactggtgttcttgttctgatttgcgcacttgttggtgctgggtttgcatgggaacgtcatcagctaaagcaagccatagagaaaattggcagtcttgatcaggctgttaaggaacgtgataagtcaataatggatcttaaccagaccattgagacgatgaacaaagcagagcaacattttcacagccaggaagtgaaaaatgaatcagaacaagccaagtatgctgacaggcaaatggaacgaaaagctgaagttcagaaacaactggttgcggcgggtaatgttcgccagcgtattcctgctgacactcagcggttgctccgggagtcgatcagcgaatttaacgccgacgccgacaaaggttaaccaccctgcccccaaaagtgcatttatgtgtaggatgccagagtttagcagtgaatattttgatgatctgccagcgtatatcctcgatacagaaacgatgctgatggggattaacaggaagaatcgcaacgttaatgattacaaccgagctattagcggtaactaaaagggatttttatgtctgataaagtaacagtaaagcaaactatcaacaaagcgacttcaatctacaaaattgagcaaatcactgttggcaagccaggatctgaacaataccgtcgtgctttcgagcttgccgatcagcttggtttaaaacacccggattgcattgagcatgtatttccgacctatgctgatgagcaatgtactcatgttcttaccgaagaggattttttcagcactgaagaacgagaaggcgttgatcgctgcattggtgtgatttgttcttcggtaagtgatgagttattccctaatgtgcctgaatatggtggtattggataccaattcctgtacgagggcgatgagcttaaatgctatgaacatggtcttctcatcgaaagcgtagaataatacgactcccttccaaccggctacgttggccggtttttcacttatccacattatccactggatagatccaataatcaggtccatacagatcccaattagatccatatagatccctgatcgttgcaggccgcgccacgtctggcttagaagtgtatcgcgatgtgtgctggagggaaaacgatgtgtgctggagggataaaaatgtgtgctgacgggttgctaatgtgtgctggcgggatataggatgtgtgttgacgggaaagcttgggtagttatcaccacttataaaaactatccacacaattcggaaaaagtaatatgaatcaatcatttatctccgatattctttacgcagacattgaaagtaaggcaaaagaactaacagttaattcaaacaacactgtgcagcctgtagcgttgatgcgcttgggggtattcgtgccgaagccatcaaagagcaaaggagaaagtaaagagattgatgccaccaaagcgttttcccagctggagatagctaaagccgagggttacgatgatattaaaatcaccggtcctcgactcgatatggatactgatttcaaaacgtggatcggtgtcatctacgcgttcagcaaatacggcttgtcctcaaacaccatccagttatcgtttcaggaattcgctaaagcctgtggtttcccctcaaaacgtctggatgcgaaactgcgtttaaccattcatgaatcacttggacgcttgcgtaacaagggtatcgcttttaagcgcggaaaagatgctaaaggcggctatcagactggtctgctgaaggtcgggcgttttgatgctgaccttgatctgatagagctggaggctgattcgaagttgtgggagctgttccagcttgattatcgcgttctgttgcaacaccacgccttgcgtgcccttccgaagaaagaagctgcacaagccatttacactttcatcgaaagccttccgcagaacccgttgccgctatcgttcgcgcgaatccgtgagcgcctggctttgcagtcagctgttggcgagcaaaaccgtatcattaagaaagcgatagaacagcttaaaacaatcggctatctcgactgttctattgagaagaaaggccgggaaagttttgtaatcgtccattctcgcaatccaaagctgaaactccccgaataagtgtgtgctggagggaaaccgcattaaaaagatgtgtgctgccgggaaggcttgtccaatttcctgtttttgatgtgcgctggagggggacgcccctcagtttgcccagactttccctccagcacacatctgtccatccgcttttccctccagtgcacatgtaattctctgcctttccctccagcacacatatttgataccagcgatccctccacagcacataattcaatgcgacttccctctatcgcacatcttagacttttattctccctccagcacacatcgaagctgccgggcaagccgttctcaccagttgatagagagtgaagcttggctgcccattgaagcaggaaatcaccaaaatgattcaggctacaacctgaacgtagaagaaatccgcgtcctttatgcgtggaggatgccaaagcatgttgtgacacacttggcaaaggagtaagcatgcagagaatgctatgtacaagcatctacgcatacattattattttatgcagcatttttaattaaattcaaaaatacagcataaaggatgactttcgatgagtgattccagccagcttcacaaggttgctcaaagagcaaacagaatgctcaatgttctgactgaacaagtacagttgcaaaaggatgagctacacgcgaacgagttttaccaggtctatgcgaaagcggcactggcaaaattgcctctactgactcgagcgaacgttgactatgccgtaagtgaaatggaagaaaagggttatgttttcgataaacgccctgctggctcttcaatgaaatatgcgatgtcaattcagaacatcattgacatatatgaacatcgcggagtgccaaaataccgggatcgctacagcgaagcgtatgtgattttcatctccaatcttaaaggcggtgtgtcaaaaactgtatcgacggtttctctggcgcatgcaatgcgtgctcaccctcatcttcttatggaggatttaaggattctggttattgaccttgatccgcaatcttcagcaacgatgtttttaagccataaacactctattggtatcgtaaacgcaacatctgcacaggctatgttgcagaatgtaagccgtgaagagctgttagaggagtttattgttccttctgttgtacctggggttgacgttatgcctgcgtcgattgacgatgcctttattgcatccgattggagagagctgtgcaatgagcatctaccgggtcagaacatccatgctgtcctgaaagaaaatgtgattgataagctgaagagcgattatgactttatcctcgttgatagtggtcctcaccttgacgccttcctgaaaaatgctttggcctcggccaatatactgtttacacctctgccgccagcaactgtcgatttccactcatcgcttaaatacgttgcccgccttcctgagttggtgaaactcatttcggatgaaggctgcgagtgccagcttgcgactaacattggttttatgtccaagttgagtaacaaggcagaccataagtattgccatagcctggctaaagaagtgttcggtggggatatgcttgatgtcttcctccctcgccttgacggttttgaacgctgcggcgagtcttttgacactgttatttcagctaacccggcaacgtatgttggtagtgctgatgcattgaagaacgcgcgaattgccgcggaagattttgctaaagcagtttttgaccgtat

SEQ ID NO: 7 - DNA, 7870 bp, Artificial Sequence
  Synthetic polynucleotide
gtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgcggccgcagtactctgcagattttatgcaaaattaaagtcttgtgacaacagctttctccttaagtgcaaatatcgcccattctttcctcttttcgtatataaatgctgtaatagtaggatgtcgtacccgtaaaggtacgacattgaatattaatatactcctaagtttactttcccaatatttatattaggacgtccccttcgggtaaataaattttagtggcagtggtaccgccactccctattttaatactgcgaaggaggcagttggcaggcaactcgtcgttcgcagtatataaatatccactaatatttatattcccgtaaggggacgtcccgaaggggaaggggaaagaagcagtcgcctccttgcgaaaaggtttacttgcccgaccagtgaaaagcatgctgtaagatataaatctaccctgaaagggatgcatttcaccataatactatacaaatggtgttaccctttgaggatcataacggtgctactggaatatatggtctcttcatggatagacgatagccatttatttacccattaaggggacattagtggcctgtcactgctccttacgagacgccagtggacgttcgtcctagaaaatttatgcgctgcctagaagccccaaaagggaagtttactgactcgttagagcgtgcgctaacaggtttaaatacttcaatatgtatattaggacgccggtggcagtggtaccgccactgccaccgtcggaggacgtcccttacggtatattatatactaggattttaatactccgaaggaggcagtggcggtaccactgccactaatatttatattcccgtaagggacgtcctccttcggagtatgtaaacattctaagtttacttgcccaatatttatattaggcagttggcaggcaactgctagctctcctccttcggagtatgtaaacatcgcagtatataaatatccactaatatttatattcccgtaaggggacgtcccgaaggggaaggggaaggacgtcagtggcagttgcctgccaactgcctaggcaagtaaacttaggagtatataaatataggcagtcgcggtaccactgccactgacgtcctgccaactgcctaggcaagtaaacttaagtggcactaaaatgcatttgcccgaaggggaaggaggacgccagtggcagtggtaccgccactgcctccttcggagtattaaaatcctagtatgtaaatctgctagcgcaggaaataaattttattctatttatatactccgttaggaggtaagtaaaccccttccccttcgggacgtcagtgcagttgcctgccaactgcctaatataaatattagaccactaaagtttggcaactgccaactgttgtccttcggaggaaaaaaaatggttaactcgcaagcagttaacataactaaagtttgttactttaccgaagacgtttaccctttctcggttaaggagacggagacagttgcactgtgactgcctagtatagcaattttgtttttgtttatatgctcgacaaaatgactttcataaaaatataaagtagttagctagttattttatatcactataactagggttctcagaggcaccgaagtcacttgtaaaaatagtactttttaacttgtttaatcttcgtgttcttcaaaaggatcacgtaatttttttgaaggtggaccaaaactaacataaactgaatagccagttacacttaacagaagaaaccataaaaaaaaggtaaagaaaaaagctggactttccatagctcatttaataataaaattattctcttttcaacatatctcttagatagttcaaaagacttgacgactgtgtcccacatttttaaacaaaattaatctactcaaaattttgccctgagaaagaataacttacttcgtttttgcagtagccattcatgtcactttgaaactgtccttacaaagttaaacattaattaaaaattatttaatttttatataacaaatattatattaaataaaaaatgaacaaagaacttctaagatcgtctttagtgagtaattaaagagttttacttaccagacaaggcagttttttcattcttttaaagcaggcagttctgaaggggaaaagggactgcctactgcggtcctaggtaaatacatttttatgcaatttatttcttgtgctagtaggtttctatactcacaagaagcaaccccttgacgagagaacgttatcctcagagtatttataatcctgagagggaatgcactgaagaatattttccttattttttacagaaagtaaataaaatagcgctaataacgcttaattcatttaatcaattatggcaacaggaacttctaaagctaaaccatcaaaagtaaattcagacttccaagaacctggtttagttacaccattaggtactttattacgtccacttaactcagaagcaggtaaagtattaccaggctggggtacaactgttttaatggctgtatttatccttttatttgcagcattcttattaatcattttagaaatttacaacagttctttaattttagatgacgtttctatgagttgggaaactttagctaaagtttcttaattttatttaacacaaacataaaatataaaactgtttgttaaggctagctgctaagtcttcttttcgctaaggtaaactaagcaactcaaccatatttatattcggcagtggcaccgccaactgccactggccttccgttaagataaacgcgtggatctcacgtgactagtgatatctacgtaatcgatgaattcgatcccatttttataactggatctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcgtatggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataattccaagcattatctaaaatactctgcaggcacgctagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttatttttatggtgtttaggttaaatacctaaacatcattttacatttttaaaattaagttctaaagttatcttttgtttaaatttgcctgtctttataaattacgatgtgccagaaaaataaaatcttagctttttattatagaatttatctttatgtattatattttataagttataataaaagaaatagtaacatactaaagcggatgtagcgcgtttatcttaacggaaggaattcggcgcctacgtacccgggtcgcgaggatccacgcgttaatagctcacttttctttaaatttaatttttaatttaaaggtgtaagcaaattgcctgacgagagatccacttaaaggatgacagtggcgggctactgcctacttccctccgggataaaatttatttgaaaaacgttagttacttcctaacggagcattgacatccccatatttatattaggacgtccccttcgggtaaataaattttagtggacgtccccttcgggcaaataaattttagtggacaataaataaatttgttgcctgccaactgcctaggcaagtaaacttgggagtattaaaataggacgtcagtggcagttgcctgccaactgcctatatttatatactgcgaagcaggcagtggcggtaccactgccactggcgtcctaatataaatattgggcaactaaagtttatagcagtattaacatcctatatttatatactccgaaggaacttgttagccgataggcgaggcaacaaatttatttattgtcccgtaaaaggatgcctccagcatcgaaggggaaggggacgtcctaggccataaaactaaagggaaatccatagtaactgatgttataaatttatagactccaaaaaacagctgcgttataaataacttctgttaaatatggccaaggggacaggggcactttcaactaagtgtacattaaaaattgacaattcaattttttttaattataatatatatttagtaaaatataacaaaaagcccccatcgtctaggtagaattccagctggcggccgccctatg

SEQ ID NO: 8 - DNA, 1353 bp, Saccharomyces cerevisiae
tcgcgcgtttcggtgatgacggtgaaaacctctgacacatgcagctcccggagacggtcacagcttgtctgtaagcggatgccgggagcagacaagcccgtcagggagcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcagga

SEQ ID NO: 9 - DNA, 2474 bp, Saccharomyces cerevisiae
tcctgatgcggtattttctccttacgcatctgtgcggtatttcacaccgcatatcgacggtcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttacgacgtagtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 10 - DNA, 1177 bp, Saccharomyces cerevisiae
aattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcg

SEQ ID NO: 11 - DNA, 3008 bp, Saccharomyces cerevisiae
tgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaacccccccccccccccccgatgcgccgcgtgcggctgctggagatggcggacgcgatggatatgttctgccaagggttgggtcgacgctaccttaagagagacgcgtgcggccgcaagcttgcatgcctgcaggtcgatcgactctagaaatcgatagatctgaattaattcttgaataatacataacttttcttaaaagaatcaaagacagataaaatttaagagatattaaatattagtgagaagccgagaattttgtaacaccaacataacactgacatctttaacaacttttaattatgatacatttcttacgtcatgattgattattacagctatgctgacaaatgactcttgttgcatggctacgaaccgggtaatactaagtgattgactcttgctgaccttttattaagaactaaatggacaatattatggagcatttcatgtataaattggtgcgtaaaatcgttggatctctcttctaagtacatcctactataacaatcaagaaaaacaagaaaatcggacaaaacaatcaagtatggattctagaacagttggtatattaggagggggacaattgggacgtatgattgttgaggcagcaaacaggctcaacattaagacggtaatactagatgctgaaaattctcctgccaaacaaataagcaactccaatgaccacgttaatggctccttttccaatcctcttgatatcgaaaaactagctgaaaaatgtgatgtgctaacgattgagattgagcatgttgatgttcctacactaaagaatcttcaagtaaaacatcccaaattaaaaatttacccttctccagaaacaatcagattgatacaagacaaatatattcaaaaagagcatttaatcaaaaatggtatagcagttacccaaagtgttcctgtggaacaagccagtgagacgtccctattgaatgttggaagagatttgggttttccattcgtcttgaagtcgaggactttggcatacgatggaagaggtaacttcgttgtaaagaataaggaaatgattccggaagctttggaagtactgaaggatcgtcctttgtacgccgaaaaatgggcaccatttactaaagaattagcagtcatgattgtgaggtctgttaacggtttagtgttttcttacccaattgtagagactatccacaaggacaatatttgtgacttatgttatgcgcctgctagagttccggactccgttcaacttaaggcgaagttgttggcagaaaatgcaatcaaatcttttcccggttgtggtatatttggtgtggaaatgttctatttagaaacaggggaattgcttattaacgaaattgccccaaggcctcacaactctggacattataccattgatgcttgcgtcacttctcaatttgaagctcatttgagatcaatattggatttgccaatgccaaagaatttcacatctttctccaccattacaacgaacgccattatgctaaatgttcttggagacaaacatacaaaagataaagagctagaaacttgcgaaagagcattggcgactccaggttcctcagtgtacttatatggaaaagagtctagacctaacagaaaagtaggtcacataaatattattgcctccagtatggcggaatgtgaacaaaggctgaactacattacaggtagaactgatattccaatcaaaatctctgtcgctcaaaagttggacttggaagcaatggtcaaaccattggttggaatcatcatgggatcagactctgacttgccggtaatgtctgccgcatgtgcggttttaaaagattttggcgttccatttgaagtgacaatagtctctgctcatagaactccacataggatgtcagcatatgctatttccgcaagcaagcgtggaattaaaacaattatcgctggagctggtggggctgctcacttgccaggtatggtggctgcaatgacaccacttcctgtcatcggtgtgcccgtaaaaggttcttgtctagatggagtagattctttacattcaattgtgcaaatgcctagaggtgttccagtagctaccgtcgctattaataatagtacgaacgctgcgctgttggctgtcagactgcttggcgcttatgattcaagttatacaacgaaaatggaacagtttttattaaagcaagaagaagaagttcttgtcaaagcacaaaagttagaaactgtcggttacgaagcttatctagaaaacaagtaatatataagtttattgatatacttgtacagcaaataattataaaatgatatacctattttttaggctttgttatgattacatcaaatgtggacttcatacatagaaatcaacgcttacaggtgtccttttttaagaatttcatacataagatctctcgaggatccccgggtaccgagctcgaattcgcggccgcccgcgggttaaccctagggcatgcactagtggcctaattggccgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaa

SEQ ID NO: 12 - DNA, 4879 bp, Saccharomyces cerevisiae
agcagttgctttctcctatgggaagagctttctaagtctgaagaagtaaacagttctttgctatttcacacttcctggttgatggtcacttgctgcctgaaatatatatatatgtatgacatatgtacttgttttcttttttgtgcctttgttacgtctatattcattgaaactgattattcgattttcttcttgctgaccgcttctagaggcatcgcacagttttagcgaggaaaactcttcaatagttttgccagcggaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgta

SEQ ID NO: 13 - DNA, 1475 bp, Artificial Sequence
  Synthetic polynucleotide
ttaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaata

SEQ ID NO: 14 - DNA, 931 bp, Artificial Sequence
  Synthetic polynucleotide
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 15 - DNA, 1382 bp, Artificial Sequence
  Synthetic polynucleotide
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacat

SEQ ID NO: 16 - DNA, 679 bp, Artificial Sequence
  Synthetic polynucleotide
cccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtga

SEQ ID NO: 17 - DNA, 875 bp, Artificial Sequence
  Synthetic polynucleotide
ttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttcatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtagtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccatcggca

SEQ ID NO: 18 - DNA, 1582 bp, Artificial Sequence
  Synthetic polynucleotide
actagtatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcatatggtaccagtatctttcacaagtcttttagcagcatctccaccttcacgtgcaagttgccgtccagctgctgaagtggaatcagttgcagtagaaaaacgtcaaacaattcaaccaggtacaggttacaataacggttacttttattcttactggaatgatggacacggtggtgttacatatactaatggacctggtggtcaatttagtgtaaattggagtaactcaggcaattttgttggaggaaaaggttggcaacctggtacaaagaataaggtaatcaatttctctggtagttacaaccctaatggtaattcttatttaagtgtatacggttggagccgtaacccattaattgaatattatattgtagagaactttggtacatacaacccttcaacaggtgctactaaattaggtgaagttacttcagatggatcagtttatgatatttatcgtactcaacgcgtaaatcaaccatctataattggaactgccactttctaccaatactggagtgtaagacgtaatcatcgttcaagtggtagtgttaatacagcaaaccactttaatgcatgggctcaacaaggtttaacattaggtacaatggactatcaaattgtagctgttgaaggttatttttcatcaggtagtgcttctatcactgttagcggtaccggtgaaaacttatactttcaaggctcaggtggcggtggaagtgattacaaagatgatgatgataaaggaaccggttaatctagacttagcttcaactaactctagctcaaacaactaatttttttttaaactaaaataaatctggttaaccatacctggtttattttagtttagtttatacacacttttcatatatatatacttaatagctaccataggcagttggcaggacgtccccttacgggacaaatgtatttattgttgcctgccaactgcctaatataaatattagtggacgtccccttccccttacgggcaagtaaacttagggattttaatgctccgttaggaggcaaataaattttagtggcagttgcctcgcctatcggctaacaagttccttcggagtatataaatatcctgccaactgccgatatttatatactaggcagtggcggtaccactcgacactagt

SEQ ID NO: 19 - DNA, 2017 bp, Artificial Sequence
  Synthetic polynucleotide
actagtatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcatatggtaccacacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagttgctgacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaaggtaccggtgaaaacttatactttcaaggctcaggtggcggtggaagtgattacaaagatgatgatgataaaggaaccggttaatctagacttagcttcaactaactctagctcaaacaactaatttttttttaaactaaaataaatctggttaaccatacctggtttattttagtttagtttatacacacttttcatatatatatacttaatagctaccataggcagttggcaggacgtccccttacgggacaaatgtatttattgttgcctgccaactgcctaatataaatattagtggacgtccccttccccttacgggcaagtaaacttagggattttaatgctccgttaggaggcaaataaattttagtggcagttgcctcgcctatcggctaacaagttccttcggagtatataaatatcctgccaactgccgatatttatatactaggcagtggcggtaccactcgacactagt

SEQ ID NO: 20 - DNA, 6521 bp, Artificial Sequence
  Synthetic polynucleotide
tcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataggcggccgcggcgcgccgttccggatctgcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagcttgacattgtaggacgtttaaacattaccctgttatccctaggatcctacgtatacatactccgaaggaggacaaatttatttattgtggtacaataaataagtggtacaataaataaattgtatgtaaaccccttccccttcgggacgtccccttacgggaatataaatattagtggcagttgcctgccaacaaatttatttattgtattaacataggcagtggcggtaccactgccactggcgtcctaatataaatattgggcaactaaagtttatcgcagtattaacataggcagtggcggtaccactgccactggcgtcctccttcggagtatgtaaacctgctaccgcagcaaataaattttattctattttaatactacaatatttagattcccgttaggggataggccaggcaattgtcactggcgtcatagtatatcaatattgtaacagattgacaccctttaagtaaacattttttttaggattcatatgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcaatgacaattgcgatcggtacatatcaagagaaacgcacatggttcgatgacgctgatgactggcttcgtcaagaccgtttcgtattcgtaggttggtcaggtttattactattcccttgtgcttactttgcaactccggtccggcggccgcctcgagacgcttaccagacaaggcagttttttcattcttttaaagcaggcagttctgaaggggaaaagggactgcctactgcggtcctaggtaaatacatttttatgcaatttatttcttgtgctagtaggtttctatactcacaagaagcaaccccttgacgagagaacgttatcctcagagtatttataatcctgagagggaatgcactgaagaatattttccttattttttacagaaagtaaataaaatagcgctaataacgcttaattcatttaatcaattatggcaacaggaacttctaaagctaaaccatcaaaagtaaattcagacttccaagaacctggtttagttacaccattaggtactttattacgtccacttaactcagaagcaggtaaagtattaccaggctggggtacaactgttttaatggctgtatttatccttttatttgcagcattcttattaatcattttagaaatttacaacagttctttaattttagatgacgtttctatgagttgggaaactttagctaaagtttcttaattttatttaacacaaacataaaatataaaactgtttgttaaggctagctgctaagtcttcttttcgctaaggtaaactaagcaactcaaccatatttatattcggcagtggcaccgccactgccactggccttccgttaagataaacgcgttaatagctcacttttctttaaatttaatttttaatttaaaggtgtaagcaaattgcctgacgagagatccacttaaaggatgacagtggcgggctactgcctacttccctccgggataaaatttatttgaaaaacgttagttacttcctaacggagcattgacatccccatatttatattaggacgtccccttcgtcgacattaccctgttatccctaggccggcctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgttaattaagcggccgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccg

SEQ ID NO: 21 - DNA, 46 bp, Artificial Sequence
  Primer
ttggttgcggccgcttaattaacgatgtatattttcctgtacaatc

SEQ ID NO: 22 - DNA, 58 bp, Artificial Sequence
  Primer
gtttaaacattaccctgttatccctaggccggcctaagaaaccattattatcatgaca

SEQ ID NO: 23 - DNA, 58 bp, Artificial Sequence
  Primer
ggccggcctagggataacagggtaatgtttaaacgtcctacaatgtcaagctcgaccg

SEQ ID NO: 24 - DNA, 46 bp, Artificial Sequence
  Primer
ttggttgcggccgcggcgcgccgttccggatctgcatcgcaggatg

SEQ ID NO: 25 - DNA, 54 bp, Artificial Sequence
  Primer
ttggtttagggataacagggtaatgtcgacaacatgctaagtttacttgcccga

SEQ ID NO: 26 - DNA, 52 bp, Artificial Sequence
  Primer
actccggtccggcggccgcctcgagacgacttgtccgcttcatcagacacgg

SEQ ID NO: 27 - DNA, 52 bp, Artificial Sequence
  Primer
cgtctcgaggcggccgccggaccggagttgcaaagtaagcacaagggaatag

SEQ ID NO: 28 - DNA, 59 bp, Artificial Sequence
  Primer
tggttattaccctgttatccctaggatcctacgtatacatactccgaaggaggacaaat

SEQ ID NO: 29 - DNA, 48 bp, Artificial Sequence
  Primer
cgtctcgaggcggccgccggaccggagtcatccgccccatctaataaa

SEQ ID NO: 30 - DNA, 56 bp, Artificial Sequence
  Primer
ttggttattaccctgttatccctaggatcctacgtacagtggcggtaccacaataa

SEQ ID NO: 31 - DNA, 53 bp, Artificial Sequence
  Primer
ttggtttagggataacagggtaatgtcgacgtatgtaaaccccttcgggcaac

SEQ ID NO: 32 - DNA, 50 bp, Artificial Sequence
  Primer
actccggtccggcggccgcctcgagacgccgtaaacagataggaatgacg

SEQ ID NO: 33 - DNA, 48 bp, Artificial Sequence
  Primer
cgtctcgaggcggccgccggaccggagtgaatccaggcatcttgggta

SEQ ID NO: 34 - DNA, 56 bp, Artificial Sequence
  Primer
ttggttattaccctgttatccctaggatcctacgtagggaaaggtgcaactacctg

SEQ ID NO: 35 - DNA, 50 bp, Artificial Sequence
  Primer
ttggtttagggataacagggtaatgtcgacaacatgcttggcactggttt

SEQ ID NO: 36 - DNA, 54 bp, Artificial Sequence
  Primer
actccggtccggcggccgcctcgagacgtggtaatttatttggtaatttggtca

SEQ ID NO: 37 - DNA, 48 bp, Artificial Sequence
  Primer
cgtctcgaggcggccgccggaccggagtaaccaaccattgtgtgacca

SEQ ID NO: 38 - DNA, 56 bp, Artificial Sequence
  Primer
ttggttattaccctgttatccctaggatcctacgtaagagtacgggatgtgggatg

SEQ ID NO: 39 - DNA, 53 bp, Artificial Sequence
  Primer
ttggtttagggataacagggtaatgtcgacccataagtaaactcccttttgga

SEQ ID NO: 40 - DNA, 50 bp, Artificial Sequence
  Primer
actccggtccggcggccgcctcgagacgtaaaattgtttgtgtggtctgg

SEQ ID NO: 41 - DNA, 52 bp, Artificial Sequence
  Primer
cgtctcgaggcggccgccggaccggagtaaatgtaacttttgttgtcgatcc

SEQ ID NO: 42 - DNA, 60 bp, Artificial Sequence
  Primer
ttggttattaccctgttatccctaggatcctacgtagggtaaataaattttagtggacgt

SEQ ID NO: 43 - DNA, 54 bp, Artificial Sequence
  Primer
ttggtttagggataacagggtaatgtcgacgaaggggacgtcctaatataaata

SEQ ID NO: 44 - DNA, 50 bp, Artificial Sequence
  Primer
actccggtccggcggccgcctcgagacgcttaccagacaaggcagttttt

SEQ ID NO: 45 - DNA, 48 bp, Artificial Sequence
  Primer
cgtctcgaggcggccgccggaccggagtgctgctggttatgcagttga

SEQ ID NO: 46 - DNA, 56 bp, Artificial Sequence
  Primer
ttggttattaccctgttatccctaggatcctacgtagaggaccaaatcctgcgtta

SEQ ID NO: 47 - DNA, 50 bp, Artificial Sequence
  Primer
ttggtttagggataacagggtaatgtcgactttgcttgcctacaagagca

SEQ ID NO: 48 - DNA, 48 bp, Artificial Sequence
  Primer
actccggtccggcggccgcctcgagacgttgcattaaaatccggaagg

SEQ ID NO: 49 - DNA, 36 bp, Artificial Sequence
  Primer
ttggttcccgggtcgcgcgtttcggtgatgacggtg

SEQ ID NO: 50 - DNA, 42 bp, Artificial Sequence
  Primer
ttggttgtcgacccgcggtgatgcggtattttctccttacgc

SEQ ID NO: 51 - DNA, 36 bp, Artificial Sequence
  Primer
ttggttcccgggtcctgatgcggtattttctcctta

SEQ ID NO: 52 - DNA, 36 bp, Artificial Sequence; identical to SEQ ID NO: 49
  Primer

SEQ ID NO: 53 - DNA, 36 bp, Artificial Sequence
  Primer
ttggttcccgggaagcttgcatgcctgcaggtcgat

SEQ ID NO: 54 - DNA, 36 bp, Artificial Sequence
  Primer
ttggttcccgggagcagttgctttctcctatgggaa

SEQ ID NO: 55 - DNA, 36 bp, Artificial Sequence
  Primer
ttggttcccgggttaggtctagagatctgtttagct

SEQ ID NO: 56 - DNA, 50 bp, Artificial Sequence
  Primer
ttggttgtcgacggccggccactagttcgcgcgtttcggtgatgacggtg

SEQ ID NO: 57 - DNA, 50 bp, Artificial Sequence
  Primer
ttggttgtcgacggccggccactagttgatgcggtattttctccttacgc

SEQ ID NO: 58 - DNA, 50 bp, Artificial Sequence
  Primer
ttggttgtcgacggccggccactagtgatcctcgagagatcttatgtatg

SEQ ID NO: 59 - DNA, 50 bp, Artificial Sequence
  Primer
ttggttgtcgacggccggccactagttacaataaaccaagatgaagctgc

SEQ ID NO: 60 - DNA, 50 bp, Artificial Sequence
  Primer
ttggttgtcgacggccggccactagttattaagggttctcgagagctcgt

SEQ ID NO: 61 - DNA, 39 bp, Artificial Sequence
  Primer
ttggttcccggggatatcaatacattcaaatatgtatcc

SEQ ID NO: 62 - DNA, 41 bp, Artificial Sequence
  Primer
ttggttcccggggatatcatccttttaaattaaaaatgaag

SEQ ID NO: 63 - DNA, 42 bp, Artificial Sequence
  Primer
ttggttcccggggatatcttctcatgtttgacagcttatcat

SEQ ID NO: 64 - DNA, 42 bp, Artificial Sequence
  Primer
aaccaacccggggatatcatgttctgccaagggttggtttgc

SEQ ID NO: 65 - DNA, 42 bp, Artificial Sequence
  Primer
ttggttcccggggatatccccaatccaggtcctgaccgttct

SEQ ID NO: 66 - DNA, 42 bp, Artificial Sequence
  Primer
aaccaacccggggatatctcacttattcaggcgtagcaacca

SEQ ID NO: 67 - DNA, 42 bp, Artificial Sequence
  Primer
ttggttcccggggatatcttgccgggtgacgcacaccgtgga

SEQ ID NO: 68 - DNA, 42 bp, Artificial Sequence
  Primer
aaccaacccggggatatctgccgatggccgcggcgttgtgac

SEQ ID NO: 69 - DNA, 23 bp, Artificial Sequence
  Primer
catactactcagtgcagcttcac

SEQ ID NO: 70 - DNA, 25 bp, Artificial Sequence
  Primer
gtgaaggagcatgttcggcacacag

SEQ ID NO: 71 - DNA, 25 bp, Artificial Sequence
  Primer
ctgtgtgccgaacatgctccttcac

SEQ ID NO: 72 - DNA, 24 bp, Artificial Sequence
  Primer
ttgtcatattactagttggtgtgg

SEQ ID NO: 73 - DNA, 24 bp, Artificial Sequence
  Primer
ggtcggcgacaactcaatcgacag

SEQ ID NO: 74 - DNA, 23 bp, Artificial Sequence
  Primer
caacggatgttttattgcctttg

SEQ ID NO: 75 - DNA, 24 bp, Artificial Sequence
  Primer
gcaaggattttcttaacttcttcg

SEQ ID NO: 76 - DNA, 25 bp, Artificial Sequence
  Primer
atgaagtcctttgttactgtgccgc

SEQ ID NO: 77 - DNA, 23 bp, Artificial Sequence
  Primer
caggaattcgctaaagcctgtgg

SEQ ID NO: 78 - DNA, 24 bp, Artificial Sequence
  Primer
gagaataaaagtctaagatgtgcg

SEQ ID NO: 79 - DNA, 24 bp, Artificial Sequence
  Primer
gacatatatgaacatcgcggagtg

SEQ ID NO: 80 - DNA, 24 bp, Artificial Sequence
  Primer
cttcaatgcatcagcactaccaac

SEQ ID NO: 81 - DNA, 23 bp, Artificial Sequence
  Primer
ggtcagattgccctgtcgttctc

SEQ ID NO: 82 - DNA, 24 bp, Artificial Sequence
  Primer
cagtttcatttgatgctcgatgag

SEQ ID NO: 83 - DNA, 25 bp, Artificial Sequence
  Primer
ggtagaattgtccgttagttgttta

SEQ ID NO: 84 - DNA, 25 bp, Artificial Sequence
  Primer
aacagacgtagtaagaaccaccagc

SEQ ID NO: 85 - DNA, 25 bp, Artificial Sequence
  Primer
ctccagaagcgttaatgtctggctt

SEQ ID NO: 86 - DNA, 24 bp, Artificial Sequence
  Primer
cgaccatcagggacagcttcaagg

SEQ ID NO: 87 - DNA, 20 bp, Artificial Sequence
  Primer
ggtctccagaacttgctgct

SEQ ID NO: 88 - DNA, 20 bp, Artificial Sequence
  Primer
cctatcccctaacgggaatc

SEQ ID NO: 89 - DNA, 22 bp, Artificial Sequence
  Primer
agattttgtgtaatgccgaagt

SEQ ID NO: 90 - DNA, 20 bp, Artificial Sequence
  Primer
tgccgtaatcattgaccaga

SEQ ID NO: 91 - DNA, 20 bp, Artificial Sequence
  Primer
ggtgcgtaaaatcgttggat

SEQ ID NO: 92 - DNA, 20 bp, Artificial Sequence
  Primer
tttttcggcgtacaaaggac

SEQ ID NO: 93 - DNA, 20 bp, Artificial Sequence
  Primer
ctcgcctatcggctaacaag

SEQ ID NO: 94 - DNA, 20 bp, Artificial Sequence
  Primer
cacaagaagcaaccccttga

SEQ ID NO: 95 - DNA, 23 bp, Artificial Sequence
  Primer
aaatttaacgtaacgatgagttg

SEQ ID NO: 96 - DNA, 23 bp, Artificial Sequence
  Primer
gcactacctgatgaaaaataacc

SEQ ID NO: 97 - DNA, 23 bp, Artificial Sequence
  Primer
ggaaggggacgtaggtacataaa

SEQ ID NO: 98 - DNA, 23 bp, Artificial Sequence
  Primer
ttagaacgtgttttgttcccaat

SEQ ID NO: 99 - DNA, 22 bp, Artificial Sequence
  Primer
cgttcttctgagaaatggctta

SEQ ID NO: 100 - DNA, 20 bp, Artificial Sequence
  Primer
ttacgcccttgctctgattt

SEQ ID NO: 101 - DNA, 45 bp, Artificial Sequence
  Primer
caattttcttgcccaaaagctgcaggcagttctaaaaaagtccat

SEQ ID NO: 102 - DNA, 45 bp, Artificial Sequence
  Primer
tatggacttttttagaactgcctgcagcttttgggcaagaaaatt

SEQ ID NO: 103 - DNA, 20 bp, Artificial Sequence
  Primer
actggagccatttcaaggtg

SEQ ID NO: 104 - DNA, 36 bp, Artificial Sequence
  Primer
ttggttgggcggccgcttacgcccttgctctgattt

SEQ ID NO: 105 - DNA, 36 bp, Artificial Sequence
  Primer
ttggttgggcggccgcactggagccatttcaaggtg

SEQ ID NO: 106 - DNA, 24 bp, Artificial Sequence
  Primer
tagctcatccatcaagactggtaa

SEQ ID NO: 107 - DNA, 45 bp, Artificial Sequence
  Primer
cagtttccgaataactgtcgacttctccgttaagtgcatttactt

SEQ ID NO: 108 - DNA, 23 bp, Artificial Sequence
  Primer
gtattacaaaacaaagtaaatgc

SEQ ID NO: 109 - DNA, 24 bp, Artificial Sequence
  Primer
accaacctttaaactttgatacgg

SEQ ID NO: 110 - DNA, 40 bp, Artificial Sequence
  Primer
ttggttgggcggccgctagctcatccatcaagactggtaa

SEQ ID NO: 111 - DNA, 40 bp, Artificial Sequence
  Primer
ttggttgggcggccgcaccaacctttaaactttgatacgg

SEQ ID NO: 112 - DNA, 27 bp, Artificial Sequence
  Primer
ccattaggtcttactagtgtaacagca

SEQ ID NO: 113 - DNA, 24 bp, Artificial Sequence
  Primer
ctttgcagaggctagcagaattac

SEQ ID NO: 114 - DNA, 24 bp, Artificial Sequence
  Primer
gtggaagagatgaaggttacgatt

SEQ ID NO: 115 - DNA, 24 bp, Artificial Sequence
  Primer
cttgccgcagtattaacatcctat

SEQ ID NO: 116 - DNA, 22 bp, Artificial Sequence
  Primer
ctgcggtagcaggtttacatac

SEQ ID NO: 117 - DNA, 19 bp, Artificial Sequence
  Primer
gcgataatggcctgcttct

SEQ ID NO: 118 - DNA, 25 bp, Artificial Sequence
  Primer
tgcaataaataaatttgtcccctta

SEQ ID NO: 119 - DNA, 20 bp, Artificial Sequence
  Primer
tgtcccacctgcttctgaat

SEQ ID NO: 120 - DNA, 60 bp, Artificial Sequence
  Primer
cgcttgcttgttacgaagtaaagcatcgagtaaattaattgggatatcttgccgggtgac

SEQ ID NO: 121 - DNA, 60 bp, Artificial Sequence
  Primer
tcctttttcagaaatatgcatatctccttgggataagtgatcttacaataaaccaagatg

SEQ ID NO: 122 - DNA, 24 bp, Artificial Sequence
  Primer
gtatttactactttacgcgcagca

SEQ ID NO: 123 - DNA, 24 bp, Artificial Sequence
  Primer
ttgtctacaacaacttcaccgttt

SEQ ID NO: 124 - DNA, 24 bp, Artificial Sequence
  Primer
agttcctggttctgcattattagc

SEQ ID NO: 125 - DNA, 24 bp, Artificial Sequence
  Primer
tagcaattgggttgatcagttcta

SEQ ID NO: 126 - DNA, 24 bp, Artificial Sequence
  Primer
gggacgtaaaatagcagtaagcat

SEQ ID NO: 127 - DNA, 24 bp, Artificial Sequence
  Primer
tatgttcacgcccattataaacac

SEQ ID NO: 128 - DNA, 21 bp, Artificial Sequence
  Primer
tttattgttgcctgccaactg

SEQ ID NO: 129 - DNA, 21 bp, Artificial Sequence
  Primer
cacgcattccttagtgtggat

SEQ ID NO: 130 - DNA, 24 bp, Artificial Sequence
  Primer
tcagccatacttaacaccaaaaga

SEQ ID NO: 131 - DNA, 24 bp, Artificial Sequence
  Primer
gcaaactgccacatatctacaaac

SEQ ID NO: 132 - DNA, 24 bp, Artificial Sequence
  Primer
ttaatgggaaatgacctgattttt

SEQ ID NO: 133 - DNA, 24 bp, Artificial Sequence
  Primer
aagacatccctgtaagagaaatgc

SEQ ID NO: 134 - DNA, 24 bp, Artificial Sequence
  Primer
aatttattggcacgaagggtatta

SEQ ID NO: 135 - DNA, 24 bp, Artificial Sequence
  Primer
ctacccatttagttccttcgctta

SEQ ID NO: 136 - DNA, 24 bp, Artificial Sequence
  Primer
tgtgtggtagacccaaattaaatg

SEQ ID NO: 137 - DNA, 24 bp, Artificial Sequence
  Primer
tcttggatatctgggagtaagagg

SEQ ID NO: 138 - DNA, 20 bp, Artificial Sequence
  Primer
gtacgctgctagcgaaatcc

SEQ ID NO: 139 - DNA, 20 bp, Artificial Sequence
  Primer
taggacggcagtggcaagta

SEQ ID NO: 140 - DNA, 20 bp, Artificial Sequence
  Primer
aacgtagctgaaggggaaaa

SEQ ID NO: 141 - DNA, 20 bp, Artificial Sequence
  Primer
cattggccattgcttgatta

SEQ ID NO: 142 - DNA, 20 bp, Artificial Sequence
  Primer
atactgaacgggcagtttgc

SEQ ID NO: 143 - DNA, 20 bp, Artificial Sequence
  Primer
tcacttgttcgatgccatgt

SEQ ID NO: 144 - DNA, 20 bp, Artificial Sequence
  Primer
ccaagctcaaaagaagcaca

SEQ ID NO: 145 - DNA, 20 bp, Artificial Sequence
  Primer
cttttccttttcgggcaaac

SEQ ID NO: 146 - DNA, 24 bp, Artificial Sequence
  Primer
agctctttacacacaccaccaata

SEQ ID NO: 147 - DNA, 24 bp, Artificial Sequence
  Primer
gctacgttaccttgccataaagtt

SEQ ID NO: 148 - DNA, 20 bp, Artificial Sequence
  Primer
gcagttgcctcctttacagt

SEQ ID NO: 149 - DNA, 21 bp, Artificial Sequence
  Primer
atttttccgtaggaggaacga

SEQ ID NO: 150 - DNA, 24 bp, Artificial Sequence
  Primer
cctgatttaacgattactggaatg

SEQ ID NO: 151 - DNA, 24 bp, Artificial Sequence
  Primer
acctgtaggcggttatatactcca

SEQ ID NO: 152 - DNA, 20 bp, Artificial Sequence
  Primer
ccgaactgaggttgggttta

SEQ ID NO: 153 - DNA, 20 bp, Artificial Sequence
  Primer
gggggagcgaataggattag

SEQ ID NO: 154 - DNA, 21 bp, Artificial Sequence
  Primer
cgtataaaccccgaaggaaac

SEQ ID NO: 155 - DNA, 21 bp, Artificial Sequence
  Primer
caaaacggtggatttacatgg

SEQ ID NO: 156 - DNA, 21 bp, Artificial Sequence
  Primer
ccaacctacgaatacgaaacg

SEQ ID NO: 157 - DNA, 21 bp, Artificial Sequence
  Primer
caattgtcactggcgtcatag

SEQ ID NO: 158 - DNA, 20 bp, Artificial Sequence
  Primer
ttccacgtcctgtgtggtta

SEQ ID NO: 159 - DNA, 20 bp, Artificial Sequence
  Primer
aaattcggtaaaagccacca

SEQ ID NO: 160 - DNA, 24 bp, Artificial Sequence
  Primer
aaactactctcaaattgcgcttct

SEQ ID NO: 161 - DNA, 24 bp, Artificial Sequence
  Primer
taaccataatgcatcattcaaacc

SEQ ID NO: 162 - DNA, 20 bp, Artificial Sequence
  Primer
aaaaaccactgcctttggtg

SEQ ID NO: 163 - DNA, 20 bp, Artificial Sequence
  Primer
ggctacgtctccgaactcac

SEQ ID NO: 164 - DNA, 20 bp, Artificial Sequence
  Primer
gcatggacaggtgttgattg

SEQ ID NO: 165 - DNA, 20 bp, Artificial Sequence
  Primer
cgcttttagcaactttgacg

SEQ ID NO: 166 - DNA, 60 bp, Artificial Sequence
  Primer
ctactatgagtacctaaccggtcatcccacatcccgtactctcgaaacgcgcgacccggg

SEQ ID NO: 167 - DNA, 60 bp, Artificial Sequence
  Primer
cgggcatttttattaattatccaaaagggagtttacttatggggaatcatagtttcatga

SEQ ID NO: 168 - DNA, 21 bp, Artificial Sequence
  Primer
tctgtgatcgaacccattctc

SEQ ID NO: 169 - DNA, 21 bp, Artificial Sequence
  Primer
aatacgcccggtagtgatctt

SEQ ID NO: 170 - DNA, 21 bp, Artificial Sequence
  Primer
gaacaccgcatttggtttgta

SEQ ID NO: 171 - DNA, 21 bp, Artificial Sequence
  Primer
gaatttccttacgggcatttt

(19) United States
(12) Patent Application Publication
(10) Pub. No.: US 2009/0269816 A1
(43) Pub. Date: Oct. 29, 2009

(54) System for capturing and modifying large pieces of genomic DNA and constructing organisms with synthetic chloroplasts

(75) Inventors: MICHAEL MENDEZ, DEL MAR, CA (US); BRYAN O'NEILL, SAN DIEGO, CA (US); KARI MIKKELSON, SAN DIEGO, CA (US)

Correspondence Address: WILSON, SONSINI, GOODRICH & ROSATI, 650 PAGE MILL ROAD, PALO ALTO, CA, 94304-1050, US

(73) Assignee: Sapphire Energy, Inc., San Diego, CA (US)

(21) Appl. No.: 12/384,893

(22) Filed: Apr. 8, 2009

Related U.S. Application Data
(63) Continuation-in-part of application No. 12/287,230, filed on Oct. 6, 2008.
(60) Provisional application No. 60/978,024, filed on Oct. 5, 2007.

(51) Int. Cl. C12P 19/34; C12N 1/12

(52) U.S. Cl. 435/91.4; 435/257.2

(57) ABSTRACT

The functional analysis of genes frequently requires the manipulation of large genomic regions. A yeast-bacteria shuttle vector is described that can be used to clone large regions of DNA by homologous recombination. Also described is a method for isolating entire genomes, including chloroplast genomes, or large portions thereof, and manipulating the same. Also described are methods for determining minimal genomes, minimal pathway requirements, and minimal organelle genomes.

### CROSS-REFERENCE TO RELATED APPLICATIONS

This application is a continuation in part and claims the benefit of priority of co-pending U.S. patent application Ser. No. 12/287,230 (filed Oct. 6, 2007) which claims priority to and benefit of U.S. Provisional Application No. 60/978,024 (filed Oct. 5, 2007), now abandoned, each of which application is incorporated herein by reference.

### BACKGROUND

For the functional analysis of many genes, investigators need to isolate and manipulate large DNA fragments. The advent of genomics and the study of genomic regions of DNA have generated a need for vectors capable of carrying large DNA regions.

In general, two types of yeast vector systems are presently available. The first type of vector is one capable of transferring small insert DNA between yeast and bacteria. A second type of vector is a fragmenting vector which creates interstitial or terminal deletions in yeast artificial chromosomes (YACs). The small insert shuttle vectors are able to recombine with and recover homologous sequences. They are centromere-based and replicate stably and autonomously in yeast, but also contain a high-copy origin of replication for maintenance as bacterial plasmids. However, these vectors are limited by their small insert capacity. The second type of vector (also known as fragmenting vectors) has recombinogenic sequences, but is unable to transfer the recovered insert DNA to bacteria for large preparations of DNA.

Researchers use fragmentation techniques to narrow down the region of interest in YACs. However, isolating sufficient quantities of YAC DNA from agarose gels for microinjection or electroporation remains cumbersome. Purification remains a problem when the YAC comigrates with an endogenous chromosome. In addition, YACs may be chimeric or contain additional DNA regions that are not required for the particular functional study.

Types of vectors available for cloning large fragments in bacteria are cosmids, P1s and bacterial artificial chromosomes (BACs). These vectors are limited to bacteria and cannot be shuttled to yeast for modification by homologous recombination. Bacterial vectors are also limited in their use for transforming plants and algae. For example, though chloroplasts are thought to originate from the endosymbiosis of photosynthetic bacteria into eukaryotic hosts translation of chloroplasts in more complex. Adding to the complexity of genetically engineering plants and algae is the presence of multiple chloroplasts with multiple copies of the chloroplast genome. Thus, there exists a need for developing a method to express proteins from large fragments of DNA in the chloroplasts of plants and algae

### SUMMARY

The present disclosure relates to compositions and methods of isolating, characterizing, and/or modifying large DNA, including entire genomes of bacteria and chloroplasts. The compositions include shuttle vectors into which target DNA may be inserted. The methods include modifying or manipulating target DNA by removing, adding or rearranging portions and introducing the modified DNA into a host.

One aspect of the present disclosure provides an isolated vector comprising a yeast element, a bacterial origin of replication, and at least 20 kb genomic DNA. In some vectors, the yeast element is a yeast centromere, a yeast autonomous replicating sequence, yeast auxotrophic marker, or a combination thereof. The DNA may be from a non-vascular photosynthetic organism, for example a macroalgae, microalgae, Ch. vulgaris, C. reinhardtii, D. salina, S. quadricanda or H. pluvalis. In some embodiments, the genomic DNA is modified, for example by insertion of a heterologous or homologous polynucleotide, deletion of one or more nucleic acid bases, mutation of one or more nucleic acid bases, rearrangement of one or more polynucleotides, or a combination thereof. In some instances, the modification is synthetic. Vectors of the present disclosure, when transformed into a host cell, may result in production of a product not naturally produced by the host cell. Some examples of such products include biomass-degrading enzymes, a fatty acids, terpenes or terpenoids. In some host cells, expression of the vector results in an increase production of a product naturally produced by said host cell, for example, a biomass-degrading enzyme, a terpene or a terpenoid. The vectors of the present disclosure may further comprise one or more selection markers, for example, a yeast marker, a yeast antibiotic resistance marker, a yeast auxotrophic marker, a bacterial marker, a bacterial antibiotic resistance marker, a bacterial auxotrophic marker, an algae marker, an algae antibiotic resistance marker, an algae auxotrophic marker, or a combination thereof. Vectors of the present disclosure may also contain chloroplast genomic DNA which comprises 1) 1-200 genes; 2) all essential chloroplast genes; and/or 3) 30-400 kb.

Also described herein is a host cell comprising the vectors described herein. Exemplary host cells may be naturally non-photosynthetic or photosynthetic and include, for example, Saccharomyces cerevisiae, Escherichia coli, macroalgae, microalgae, Ch. vulgaris, C. reinhardtii, D. salina, S. quadricanda or H. pluvalis.

Another aspect provides, a method for producing a vector where the method involves inserting targeting DNA into a vector—where the vector comprises a yeast centromere, a yeast autonomous replicating sequence, and a bacterial origin of replication, transforming an organism with the vector and capturing a portion of a chloroplast genome, thus producing a vector with a portion of a chloroplast genome. In some instances, the targeting DNA is chloroplast genomic DNA. This method may be used to capture a portion of a genome which is 10-400 kb in length. In some instances, the capturing step occurs by recombination. The captured portion of a chloroplast genome may be co-transformed into an organism with a vector, thus the recombination step may occur in vivo. Organisms used to practice methods disclosed herein may be eukaryotic and/or photosynthetic. In some instances, the organism is a non-vascular photosynthetic organism, for example a macroalgae, microalgae, Ch. vulgaris, C. reinhardtii, D. salina, S. quadricanda or H. pluvalis. Organisms used to practice methods disclosed herein may also be non-photosynthetic, for example yeast. In some instances, a non-photosynthetic organism may contain exogenous chloroplast DNA. In some embodiments, an additional step of modifying a portion of a chloroplast genome is utilized. A modification may be achieved through homologous recombination. Such recombination may occur in an organism, for example a eukaryotic and/or photosynthetic organism. In some instances, the organism is a non-vascular photosynthetic organism, for example a macroalgae, microalgae, Ch. vulgaris, C. reinhardtii, D. salina, S. quadricanda or H. pluvalis. In other instances, the organism may be non-photosynthetic, for example a yeast. In embodiments with a modification step, the step may comprise addition of a polynucleotide, deletion of one or more nucleic acid bases, mutation of one or more nucleic acid bases, rearrangement or a polynucleotide, or combination thereof.

Further disclosed herein is an isolated vector comprising essential chloroplast genes, a selectable marker and a manipulation in one or more nucleic acids in the vector. In some instances, essential chloroplast genes are cloned from a non-vascular photosynthetic organism such as macroalgae, microalgae, Ch. vulgaris, C. reinhardtii, D. salina, S. quadricanda or H. pluvalis. Essential chloroplast genes for use in the vectors described herein may be synthetic. The vectors described herein may further comprise an expression cassette, which may further comprise a region for integration into target DNA, for example organelle DNA. The vectors described herein may also contain one or more selection markers, for example, an auxotrophic marker, an antibiotic resistance marker, a chloroplast marker, or combinations thereof. In some instances, the essential chloroplast genes are those required for chloroplast function, photosynthesis, carbon fixation, production of one or more hydrocarbons, or a combination thereof. Essential chloroplast genes may comprise up to 200 genes and/or consist of up to 400 kb. In some of the vectors described herein a manipulation in one or more nucleic acids is an addition, deletion, mutation, or rearrangement. In some instances, expression of the vector in a host cell produces a product not naturally produced by said host cell. In other instances, expression of a vector of the present disclosure results in an increase production of a product naturally produced by said host cell. Examples of such products are biomass degrading enzymes, fatty acids, terpenes or terpenoids.

As described herein, one aspect provided is an isolated chloroplast comprising a vector of the present disclosure. In another aspect, a host cell comprising a vector of the present disclosure is provided. Host cells useful in the present disclosure may be naturally non-photosynthetic or naturally photosynthetic. Examples of useful organisms include Saccharomyces cerevisiae, Escherichia coli, macroalgae, microalgae, Ch. vulgaris, C. reinhardtii, D. salina, S. quadricanda or H. pluvalis.

In another aspect, a method is provided for transforming a cell or organism where the method comprises inserting into said cell or organism a vector comprising all essential chloroplast genes and optionally one or more genes not naturally occurring in said cell or organism. In some embodiments, the method further comprises the step of eliminating all or substantially all chloroplast genomes in said cell or organism. A cell or organism useful for this method may be photosynthetic, non-photosynthetic and/or eukaryotic. A cell or organism useful for this method may be non-vascular. In some instances, the vector for use in this method may also comprise an expression cassette and the expression cassette may be capable of integrating into non-nuclear DNA. In one embodiment the one or more genes not naturally occurring in the cell or organism is a gene in the isoprenoid pathway, MVA pathway, or MEP pathway. In another embodiment, the essential chloroplast genes are those that are required for chloroplast function, photosynthesis, carbon fixation, production of one or more hydrocarbons, or any combination thereof.

Further provided herein is a method for modifying an organism comprising the steps of transforming the organism with a vector comprising one or more polynucleotides sufficient to perform chloroplast function. In some instances, a vector useful for this method further comprises a sequence for production or secretion of a compound from said organism. In some instances, the compound is an isoprenoid. In other instances, the vector comprises all essential chloroplast genes. In still other instances, the essential chloroplast genes are rearranged or mutated. An organism useful for some embodiments comprises essentially no chloroplast genome prior to transformation.

Yet another method provided herein is a method for making a product from an organism comprising the step of transforming said organism with a vector comprising at least 20 kb of genomic DNA and one or more of the following: (i) a gene not naturally occurring in said organism; (ii) a deletion in a gene naturally occurring in said organism; (iii) a rearrangement of genes naturally occurring in said organism; and (iv) a mutation in a gene naturally occurring in said organism. In some instances, the organism is naturally photosynthetic. In other instances, the additional genes encode enzymes in the isoprenoid pathway, MVA pathway, or MEP pathway. In still another embodiment, the present disclosure provides a method for transforming a cell or organism comprising inserting into said cell or organism a chloroplast and a vector comprising all essential chloroplast genes.

The present disclosure also provides a method of producing an artificial chloroplast genome comprising the steps of: (a) providing a vector comprising one or more essential chloroplast genes; (b) adding to said vector a DNA fragment; (c) transforming a cell or organism with the vector produced by step (b); and (d) determining whether chloroplast function exists with said added DNA fragment. In some instances, the added DNA fragments comprises one or more coding regions for an enzyme in the isoprenoid, MVA or MEP pathway.

The present disclosure also provides a shuttle vector comprising a chloroplast genome. A genome may be modified. Also provided herein is a vector comprising an isolated, functional chloroplast genome. A chloroplast genome useful in such a vector may be modified.

Further provided herein is a method of producing an artificial chloroplast genome comprising the steps of: (a) providing a vector comprising all essential chloroplast genes; and (b) removing, adding, mutating, or rearranging DNA from the chloroplast genome. Such a method may further comprise the steps of transforming a redacted genome into a host organism; and (d) determining chloroplast function in the host organism. In some instances, steps (b), (c), and (d) are repeated. In still other instances, the chloroplast genome is from an organism selected from the group consisting of: macroalgae, microalgae, Ch. vulgaris, C. reinhardtii, D. salina, S. quadricanda or H. pluvalis. In other instances, the host organism is selected from the group consisting of: macroalgae, microalgae, Ch. vulgaris, C. reinhardtii, D. salina, S. quadricanda or H. pluvalis. For some embodiments, the method may further comprise the step of removing redundant DNA from a chloroplast genome. In other embodiments, the vector comprises all or substantially all of a chloroplast genome. A chloroplast genome useful in the present disclosure may be cloned from a photosynthetic organism or may be a synthetic chloroplast genome. In some instances, the vector further comprises a gene not naturally occurring in the host organism, for example a gene from the isoprenoid pathway, MVA pathway, or MEP pathway.

Yet another method provided herein is a method of producing an artificial chloroplast genome comprising the steps of: (a) providing a vector comprising an entire chloroplast genome; (b) deleting a portion of said entire chloroplast genome; and (c) determining whether chloroplast function exists without said deleted portion. In another aspect of the present disclosure, a composition comprising an isolated and functional chloroplast genome is provided. In some instances, a composition comprises a modification to said chloroplast genome.

Further provided herein is an ex vivo vector comprising a nucleic acid comprising at least about 10% of a chloroplast genome and a manipulation in one or more nucleic acids in the vector. In some instances, the nucleic acid is cloned from a non-vascular photosynthetic organism, for example a macroalgae, microalgae, Ch. vulgaris, C. reinhardtii, D. salina, S. quadricanda or H. pluvalis. In some instances, the nucleic acid is synthetic. A vector of the present disclosure may further comprise an expression cassette and an expression cassette may further comprise a region for integration into target DNA. In some instances, the target DNA is organelle DNA. A vector of the present disclosure may further comprise one or more selection markers, for example an auxotrophic marker, an antibiotic resistance marker, a chloroplast marker, or combinations thereof. In some embodiments, a manipulation in one or more nucleic acids in a vector may be an addition, deletion, mutation, or rearrangement. Expression of the vector may result in production of a product not naturally produced by a host cell and/or an increase production of a product naturally produced by a host cell. Examples of some products of the present disclosure include a terpene, terpenoid, fatty acid, or biomass degrading enzyme.

Also provided herein is an ex vivo vector comprising a nucleic acid comprising at least about 20 kilobases of a chloroplast genome and a manipulation in one or more nucleic acids in said vector. In some instances, the nucleic acid is cloned from a non-vascular photosynthetic organism, for example a macroalgae, microalgae, Ch. vulgaris, C. reinhardtii, D. salina, S. quadricanda or H. pluvalis. In some instances, the nucleic acid is synthetic. A vector of the present disclosure may further comprise an expression cassette and an expression cassette may further comprise a region for integration into target DNA. In some instances, the target DNA is organelle DNA. A vector useful in the present disclosure may further comprise one or more selection markers, for example an auxotrophic marker, an antibiotic resistance marker, a chloroplast marker, or combinations thereof. In some embodiments, a manipulation in one or more nucleic acids in a vector may be an addition, deletion, mutation, or rearrangement. Expression of the vector may result in production of a product not naturally produced by a host cell and/or an increase production of a product naturally produced by a host cell. Examples of some products of the present disclosure include a terpene, terpenoid, fatty acid, or biomass degrading enzyme.

Further provided herein is a method of producing a vector containing a reconstructed genome, comprising: introducing two or more vectors into a host cell, wherein the vectors comprise fragments of a genome, recombining the vectors into a single vector comprising at least about 90% of a genome, thereby producing a vector containing a reconstructed genome. In some instances, the host cell is eukaryotic, for example, S. cerevisiae. In other instances, the genome is an organelle genome. The organelle may be a chloroplast, for example a chloroplast from an alga, particularly a microalgae such as Ch. vulgaris, C. reinhardtii, D. salina, S. quadricanda or H. pluvalis. In some instances, the two or more vectors comprise a selectable marker. In other instances, at least one of said fragments is synthetic. In still other instances, a further step comprising modifying a portion of the genome is useful in this method. Such a modification may comprise an addition, deletion, mutation, or rearrangement. In other embodiments, the modification is the addition of an exogenous nucleic acid which results in the production or increased production of a terpene, terpenoid, fatty acid or biomass degrading enzyme.

Another aspect provides a means for inserting heterologous DNA in target regions using targeted insertion vectors produced by a one step PCR mediated method rather than cloning. Such targeted selection vectors can be used to modify DNA sequences, such as genomic DNA. In some embodiments, this method is used to insert selection markers into heterologous DNA to stabilize fragile regions and/or modify target regions. Briefly, two PCR primers are designed such that the sequence of the first approximately 40-42 nucleotides (5′->3′) of each primer are identical to the target sequences, and the final 18-20 nucleotides are identical to sequences within a vector containing the modified DNA to be inserted, for example, a selection marker cassette. The result is that during the PCR amplification, the modified DNA sequence adds flanking sequences that target the modified sequence(s) to the desired region. Such flanking sequences may also be referred to as targeting sequences. Thus a targeting sequence is a polynucleotide sequence that directs integration of a vector by homologous recombination into a particular site.

Also provided herein is a method of producing a vector containing a reconstructed genome, comprising the steps of (a) introducing two or more vectors into a host cell, wherein said vectors comprise fragments of a genome; (b) recombining said vectors into a single vector comprising at least about 90% of a genome, thereby producing a reconstructed genome. In some instances, the reconstructed genome has at least one modification. A modification can be made by homologous recombination in a yeast cell using a targeted integration vector. In some instances, the targeted integration vector comprises a segment containing the modification to be introduced and two targeting segments of 40 to 1000 bases on each end of the integration vector. In some embodiments, the targeted integration vector is produced using the polymerase chain reaction and said targeting segments are from about 40 bases to about 200 bases. In other embodiments, the targeted integration vector is made using a set of primers, each primer comprising an area of 18 to 20 bases identical to the segment of modified DNA and an area of about 40 to 200 bases identical to the targeted integration site. In other instances, the at least one modification comprises an addition, deletion, mutation or rearrangement. Fragments of the genome can be obtained from a non vascular photosynthetic organism, for example, a microalgae or a macroalgae. A photosynthetic organism useful in the present invention can be Ch. Vulgaris, C. reinhardtii D. salina, S. quadricanda and H. pluvalis. In some instances, the at least one modification introduces an exogenous coding region. In other instances, the exogenous coding region encodes an enzyme in a pathway for synthesis of a fatty acid, a terpene or a terpenoid.

Further provided herein is plant comprising a reconstituted genome produced by a method of the present invention. In some instances, the plant is a non-vascular photosynthetic organism. A non-vascular photosynthetic organism useful for the present invention can be a microalgae or a macroalgae, for example, Ch. vulgaris, C. reinhardtii, D. salina, S. quadricanda or H. pluvalis. In some instances, the plant is non-photosynthetic, which can be due to a natural or induced mutation. In some embodiments, a plant of the present invention is made photoautotrophic by the presence of the synthetic chloroplast genome.

Still further provided herein is a method for stabilizing and or modifying one or more target regions of DNA comprising: obtaining a DNA cassette comprising a selection marker, a heterologous DNA sequence or both; obtaining a set of primers, said primers comprising in a 5′ to 3′ order, approximately 40-200 nucleotides identical to a sequence in a target region to be modified and 18 to 20 nucleotides identical to a 3′ end sequence of the DNA cassette; amplifying the DNA cassette by polymerase chain reaction to produce an insertion vector comprising the DNA cassette flanked by nucleotide sequences identical to the target region to be modified or stabilized; and inserting said insertion vector into the target region of DNA by homologous recombination. In some embodiments, the primers comprise, in a 5′ to 3′ order, approximately 40-60 nucleotides identical to a sequence in the target region to be modified and 18 to 20 nucleotides identical to a 3′ end sequence of the DNA cassette.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

In the figures, the following abbreviations are used: HIS3: yeast HIS3 gene; TRP1: yeast TRP1 gene; URA3: yeast URA3 gene; ADE2: yeast ADE2 gene; LYS2: yeast LYS2 gene; TEL: yeast telomere; CEN: yeast centromere; ARS: autonomously replicating sequences, yeast origin of replication; 5FOA: 5-fluoroorotic acid; Kan: kanamycin resistance gene; P1 plasmid rep: P1 plasmid replicon; p1 lytic rep: p1 lytic replicon.

FIG. 1 provides a general description of a hybrid vector of the present disclosure. 1A Vector schematic. 1B DNA shuttling between organisms.

FIG. 2 is a schematic showing construction of a hybrid vector.

FIG. 3 is a schematic of selectable markers for modification and/or stabilization.

FIGS. 4A-4C are schematics showing sites of integration in chloroplast genome DNA. Circled numbers indicate target sites for modification. The box indicates the site targeted by the hybrid gap-filling vector.

FIG. 5 is a schematic for introduction of hybrid vector into chloroplast genome DNA.

FIG. 6 is PCR data showing integration of hybrid vector (and stabilization vector) in algae.

FIG. 7 shows analysis of captured DNA. 7A Restriction digest with EcoRI of isolated vectors containing chloroplast (L, ladder; C, parent hybrid vector; 1, Clone 1; and 2; Clone 2). 7B Restriction digest with EcoRI of isolates of Clone 1 that were passaged through yeast (L, ladder; C; 1, Clone 1; and A-M; yeast isolates). 7C Southern analysis of Clones 1 and 2 digested with EcoRI and probed with radioactive HindIII-digested total DNA from C. reinhardtii.

FIGS. 8A-8B are schematics showing architecture of isolated ex vivo vectors containing chloroplast genome DNA.

FIG. 9 shows growth of parent and transformed algae cells under various selection conditions.

FIGS. 10A-10C are schematics of restriction analysis for manipulation vectors. A) Schematic of vector architecture. B) Analysis of modified vector by restriction analysis with EcoRI

FIG. 11 shows modification of a chloroplast genome to produce a biomass-degrading enzyme. 11A PCR screen of isolated transformants. 11B Endoxylanase activity from isolated transformants.

FIG. 12 shows a PCR screen of isolated transformants modified to produce isoprenoids with the FPP synthase expression cassette targeted to site 3 (12A) or site 4 (12B).

FIG. 13 is a schematic showing capture of a partial chloroplast genome using recombination in yeast.

FIG. 14 is a schematic showing capture of an entire chloroplast genome using recombination in yeast.

FIG. 15 is a schematic showing reassembly of an entire chloroplast genome using recombination in yeast.

FIG. 16 is a schematic of a genome assembled using the method of Example 14.

FIG. 17 shows the result of PCR analysis showing proper assembly of a chloroplast genome as described in Example 14 and the targeted integration of DNA sequences as described in Example 15.

### DETAILED DESCRIPTION

While preferred embodiments of the present invention have been shown and described herein; it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Technical and scientific terms used herein have the meanings commonly understood by one of ordinary skill in the art to which the instant disclosure pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of yeast genetics useful for selected aspects of the disclosure include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This disclosure teaches methods and describes tools for capturing and modifying large pieces of DNA. It is especially useful for modifying genomic DNA, including the whole genome of an organism or organelle, or a part thereof. Novel prophetic uses of the invention are also described. The disclosure relates to the manipulation and delivery of large nucleic acids. The disclosure further relates to recombinational cloning vectors and systems and to methods of using the same.

Contemporary methods for genetically engineering genomes (e.g., chloroplast genomes) are time intensive (>1 month) and allow for only a limited number of manipulations at a time. If multiple modifications to a target genome are desired, the process must be iterated, further increasing the time required to generate a desired strain. Because metabolic engineering and/or synthetic biology require numerous modifications to a genome, these technologies are not amenable to rapid introduction of modifications to a genome. Thus, a new technology that allows for multiple modification of the chloroplast genome in a short amount of time will enable the application of metabolic engineering and/or synthetic biology to chloroplast genomes. The disclosure herein describes such technology.

The instant disclosure provides a versatile, recombinational approach to the capture, cloning, and manipulation of large nucleic acids from target cells and organelles (e.g., chloroplasts). One aspect of the present disclosure provides a recombinational cloning system. More specifically, the disclosure provides vectors, which in some embodiments, rely on homologous recombination technologies to mediate the isolation and manipulation of large nucleic acid segments. Another aspect provides methods for using such recombinational cloning vectors to clone, to manipulate and to deliver large nucleic acids to target cells and/or organelles such as chloroplasts.

In one embodiment, homologous recombination is performed in vitro. In another embodiment, homologous recombination is performed in vivo. In still another embodiment, homologous recombination occurs in an algae cell. In yet another embodiment, homologous recombination occurs in a yeast cell. In one preferred embodiment, homologous recombination occurs in Saccharomyces cerevisiae or Saccharomyces pombe. In yeast, the combination of efficient recombination processes and the availability of numerous selectable markers provides for rapid and complex engineering of target DNA sequences. Once all of the modifications are made to an ex vivo vector containing chloroplast genome DNA, the entire vector can be introduced into a chloroplast in a single transformation step. Thus, employing yeast technology will enable the application of metabolic engineering and/or synthetic biology to chloroplast genomes. One aspect of the present disclosure provides an isolated vector comprising a yeast element, a bacterial origin of replication, and at least 20 kb genomic DNA. In some vectors, the yeast element is a yeast centromere, a yeast autonomous replicating sequence, or a combination thereof. The DNA may be from a non-vascular photosynthetic organism, for example a macroalgae, microalgae, Ch. vulgaris, C. reinhardtii, D. salina, S. quadricanda or H. pluvalis. In some embodiments, the genomic DNA is modified, for example by insertion of a heterologous or homologous polynucleotide, deletion of one or more nucleic acid bases, mutation of one or more nucleic acid bases, rearrangement of one or more polynucleotides, or a combination thereof. In some instances, the modification is synthetic. Vectors of the present disclosure, when transformed into a host cell, may result in production of a product not naturally produced by the host cell. Some examples of such products include biomass-degrading enzymes, a fatty acids, terpenes or terpenoids. In some host cells, expression of the vector results in an increase production of a product naturally produced by said host cell, for example, a biomass-degrading enzyme, a terpene or a terpenoid. The vectors of the present disclosure may further comprise one or more selection markers, for example, a yeast marker, a yeast antibiotic resistance marker, a bacterial marker, a bacterial antibiotic resistance marker, an algae marker, an algae antibiotic resistance marker or a combination thereof. Vectors of the present disclosure may also contain chloroplast genomic DNA which comprises 1) 1-200 genes; 2) all essential chloroplast genes; and/or 3) 30-400 kb.

Also described herein is a host cell comprising the vectors described herein. Exemplary host cells may be naturally non-photosynthetic or photosynthetic and include, for example, Saccharomyces cerevisiae, Escherichia coli, macroalgae, microalgae, Ch. vulgaris, C. reinhardtii, D. salina, S. quadricanda or H. pluvalis.

In another aspect of the disclosure, a method for producing a vector is provided where the method involves inserting targeting DNA into a vector—where the vector comprises a yeast centromere, a yeast autonomous replicating sequence, and a bacterial origin of replication, transforming an organism with the vector and capturing a portion of a chloroplast genome, thus producing a vector with a portion of a chloroplast genome. In some instances, the targeting DNA is chloroplast genomic DNA. This method may be used to capture a portion of a genome which is 10400 kb in length. In some instances, the capturing step occurs by recombination. The captured portion of a chloroplast genome may be co-transformed into an organism with a vector, thus the recombination step may occur in vivo. Organisms used to practice methods disclosed herein may be eukaryotic and/or photosynthetic. In some instances, the organism is a non-vascular photosynthetic organism, for example a macroalgae, microalgae, Ch. vulgaris, C. reinhardtii, D. salina, S. quadricanda or H. pluvalis. Organisms used to practice methods disclosed herein may also be non-photosynthetic, for example yeast. In some instances, a non-photosynthetic organism may contain exogenous chloroplast DNA. In some embodiments, an additional step of modifying a portion of a chloroplast genome is utilized. A modification may be achieved through homologous recombination. Such recombination may occur in an organism, for example a eukaryotic and/or photosynthetic organism. In some instances, the organism is a non-vascular photosynthetic organism, for example a macroalgae, microalgae, Ch. vulgaris, C. reinhardtii, D. salina, S. quadricanda or H. pluvalis. In other instances, the organism may be non-photosynthetic, for example a yeast. In embodiments with a modification step, the step may comprise addition of a polynucleotide, deletion of one or more nucleic acid bases, mutation of one or more nucleic acid bases, rearrangement or a polynucleotide, or combination thereof.

Further disclosed herein is an isolated vector comprising essential chloroplast genes, a selectable marker and a manipulation in one or more nucleic acids in the vector. In some instances, essential chloroplast genes are cloned from a non-vascular photosynthetic organism such as macroalgae, microalgae, Ch. vulgaris, C. reinhardtii, D. salina, S. quadricanda or H. pluvalis. Essential chloroplast genes for use in the vectors described herein may be synthetic. The vectors described herein may further comprise an expression cassette, which may further comprise a region for integration into target DNA, for example organelle DNA. The vectors described herein may also contain one or more selection markers, for example, an auxotrophic marker, an antibiotic resistance marker, a chloroplast marker, or combinations thereof. In some instances, the essential chloroplast genes are those required for chloroplast function, photosynthesis, carbon fixation, production of one or more hydrocarbons, or a combination thereof. Essential chloroplast genes may comprise up to 200 genes and/or consist of up to 400 kb. In some of the vectors described herein a manipulation in one or more nucleic acids is an addition, deletion, mutation, or rearrangement. In some instances, expression of the vector in a host cell produces a product not naturally produced by said host cell. In other instances, expression of a vector of the present disclosure results in an increase production of a product naturally produced by said host cell. Examples of such products are biomass degrading enzymes, fatty acids, terpenes or terpenoids.

As described herein, one aspect of the present disclosure is an isolated chloroplast comprising a vector of the present disclosure. In another aspect, a host cell comprising a vector described herein is provided. Host cells useful in the present disclosure may be naturally non-photosynthetic or naturally photosynthetic. Examples of useful organisms include Saccharomyces cerevisiae, Escherichia coli, macroalgae, microalgae, Chlorella vulgaris, C. reinhardtii, D. salina, S. quadricanda or H. pluvalis.

Another aspect provides a method for transforming a cell or organism where the method comprises inserting into said cell or organism a vector comprising all essential chloroplast genes and optionally one or more genes not naturally occurring in said cell or organism. In one embodiment, the one or more not naturally occurring genes comprise a complete metabolic pathway. In another embodiment, the not naturally occurring metabolic pathway produces a molecule that is not metabolized by the cell or organism and preferably is not toxic to the cell or organism. In some embodiments, the method further comprises the step of eliminating substantially all chloroplast genomes in said cell or organism. A cell or organism useful for this method may be photosynthetic, non-photosynthetic and/or eukaryotic. A cell or organism useful for this method may be non-vascular. In some instances, the vector for use in this method may also comprise an expression cassette and the expression cassette may be capable of integrating into non-nuclear DNA. In one embodiment the one or more genes not naturally occurring in the cell or organism is a gene in the isoprenoid pathway, MVA pathway, or MEP pathway. In another embodiment, the essential chloroplast genes are those that are required for chloroplast function, photosynthesis, carbon fixation, production of one or more hydrocarbons, or a combination thereof.

Further provided herein is a method for modifying an organism comprising the steps of transforming the organism with a vector comprising one or more polynucleotides sufficient to perform chloroplast function. In some instances, a vector useful for this method further comprises a sequence for production or secretion of a compound from said organism. In some instances, the compound is an isoprenoid. In other instances, the vector comprises all essential chloroplast genes. In still other instances, the essential chloroplast genes are rearranged or mutated. An organism useful for some embodiments comprises essentially no chloroplast genome prior to transformation.

Yet another method provided herein is a method for making a product from an organism comprising the step of transforming said organism with a vector comprising at least 20 kb of genomic DNA and one or more of the following: (i) a gene not naturally occurring in said organism; (ii) a deletion in a gene naturally occurring in said organism; (iii) a rearrangement of genes naturally occurring in said organism; and (iv) a mutation in a gene naturally occurring in said organism. In some instances, the organism is naturally photosynthetic. In other instances, the additional genes encode enzymes in the isoprenoid pathway, MVA pathway, or MEP pathway. In still another embodiment, the present disclosure provides a method for transforming a cell or organism comprising inserting into said cell or organism a chloroplast and a vector comprising all essential chloroplast genes.

The present disclosure also provides a method of producing an artificial chloroplast genome comprising the steps of: (a) providing a vector comprising one or more essential chloroplast genes; (b) adding to said vector a DNA fragment; (c) transforming a cell or organism with the vector produced by step (b); and (d) determining whether chloroplast function exists with said added DNA fragment. In some instances, the added DNA fragments comprises one or more coding regions for an enzyme in the isoprenoid, MVA or MEP pathway.

The present disclosure also provides a shuttle vector comprising a chloroplast genome. A genome may be modified. Also provided herein is a vector comprising an isolated, functional chloroplast genome. A chloroplast genome useful in such a vector may be modified:

Further provided herein is a method of producing an artificial chloroplast genome comprising the steps of: (a) providing a vector comprising all essential chloroplast genes; and (b) removing, adding, mutating, or rearranging DNA from the chloroplast genome. Such a method may further comprise the steps of transforming a redacted genome into a host organism; and (d) determining chloroplast function in the host organism. In some instances, steps (b), (c), and (d) are repeated. In still other instances, the chloroplast genome is from an organism selected from the group consisting of: macroalgae, microalgae, Ch. vulgaris, C. reinhardtii, D. salina, S. quadricanda or H. pluvalis. In other instances, the host organism is selected from the group consisting of: macroalgae, microalgae, Ch. vulgaris, C. reinhardtii, D. salina, S. quadricanda or H. pluvalis. For some embodiments, the method may further comprise the step of removing redundant DNA from a chloroplast genome. In other embodiments, the vector comprises all or substantially all of a chloroplast genome. A chloroplast genome may be cloned from a photosynthetic organism or may be a synthetic chloroplast genome. In some instances, the vector further comprises a gene not naturally occurring in the host organism, for example a gene from the isoprenoid pathway, MVA pathway, or MEP pathway.

Yet another method provided herein is a method of producing an artificial chloroplast genome comprising the steps of: (a) providing a vector comprising an entire chloroplast genome; (b) deleting a portion of said entire chloroplast genome; and (c) determining whether chloroplast function exists without said deleted portion. In another aspect of the present disclosure, a composition comprising an isolated and functional chloroplast genome is provided. In some instances, a composition comprises a modification to said chloroplast genome.

Further provided herein is an ex vivo vector comprising a nucleic acid comprising at least about 10% of a chloroplast genome and a manipulation in one or more nucleic acids in the vector. In some instances, the nucleic acid is cloned from a non-vascular photosynthetic organism, for example a macroalgae, microalgae, Ch. vulgaris, C. reinhardtii, D. salina, S. quadricanda or H. pluvalis. In some instances, the nucleic acid is synthetic. A vector may further comprise an expression cassette and an expression cassette may further comprise a region for integration into target DNA. In some instances, the target DNA is organelle DNA. A useful vector may further comprise one or more selection markers, for example an auxotrophic marker, an antibiotic resistance marker, a chloroplast marker, or combinations thereof. In some embodiments, a manipulation in one or more nucleic acids in a vector may be an addition, deletion, mutation, or rearrangement. Expression of the vector may result in production of a product not naturally produced by a host cell and/or an increase production of a product naturally produced by a host cell. Examples of some products of the present disclosure include a terpene, terpenoid, fatty acid, or biomass degrading enzyme.

Also provided herein is an ex vivo vector comprising a nucleic acid comprising at least about 20 kilobases of a chloroplast genome and a manipulation in one or more nucleic acids in said vector. In some instances, the nucleic acid is cloned from a non-vascular photosynthetic organism, for example a macroalgae, microalgae, Ch. vulgaris, C. reinhardtii, D. salina, S. quadricanda or H. pluvalis. In some instances, the nucleic acid is synthetic. A vector may further comprise an expression cassette and an expression cassette may further comprise a region for integration into target DNA. In some instances, the target DNA is organelle DNA. A vector useful in the present disclosure may further comprise one or more selection markers, for example an auxotrophic marker, an antibiotic resistance marker, a chloroplast marker, or combinations thereof. In some embodiments, a manipulation in one or more nucleic acids in a vector may be an addition, deletion, mutation, or rearrangement. Expression of the vector may result in production of a product not naturally produced by a host cell and/or an increase production of a product naturally produced by a host cell. Examples of some products of the present disclosure include a terpene, terpenoid, fatty acid, or biomass degrading enzyme.

Further provided herein is a method of producing a vector containing a reconstructed genome, comprising: introducing two or more vectors into a host cell, wherein the vectors comprise fragments of a genome, recombining the vectors into a single vector comprising at least about 90% of a genome, thereby producing a vector containing a reconstructed genome. In some instances, the host cell is eukaryotic, for example, S. cerevisiae. In other instances, the genome is an organelle genome. The organelle may be a chloroplast, for example a chloroplast from an alga, particularly a microalgae such as Ch. vulgaris, C. reinhardtii, D. salina, S. quadricanda or H. pluvalis. In some instances, the two or more vectors comprise a selectable marker. In other instances, at least one of said fragments is synthetic. In still other instances, a further step comprising modifying a portion of the genome is useful in this method. Such a modification may comprise an addition, deletion, mutation, or rearrangement. In other embodiments, the modification is the addition of an exogenous nucleic acid which results in the production or increased production of a terpene, terpenoid, fatty acid or biomass degrading enzyme.

Large DNA Cloning and Content

An advantage of this disclosure is that it provides for the cloning, manipulation, and delivery of a vector containing chloroplast genome DNA consisting of up to all chloroplast genes (or sequences). The chloroplast genome DNA contained in the vector can be obtained by recombination of a hybrid cloning vector with one contiguous fragment of DNA or by recombination of two or more contiguous fragments of DNA.

The methods and compositions of the present disclosure may include captured and/or modified large pieces of DNA may comprise DNA from an organelle, such as mitochondrial DNA or plastid DNA (e.g., chloroplast DNA). The captured and/or modified large pieces of DNA may also comprise the entirety of an organelle's genome, e.g., a chloroplast genome. In other embodiments, the captured and/or modified large pieces of DNA comprise a portion of a chloroplast genome. A chloroplast genome may originate from any vascular or non-vascular plant, including algae, bryophytes (e.g., mosses, ferns), gymnosperms (e.g., conifers), and angiosperms (e.g., flowering plants—trees, grasses, herbs, shrubs). A chloroplast genome, or essential portions thereof, may comprise synthetic DNA, rearranged DNA, deletions, additions, and/or mutations. A chloroplast genome, or portions thereof, may comprise a one or more deletions, additions, mutations, and/or rearrangements. The deletions, additions, mutations, and/or rearrangements may be naturally found in an organism, for example a naturally occurring mutation, or may not be naturally found in nature. The chloroplast or plastid genomes of a number of organisms are widely available, for example, at the public database from the NCBI Organelle Genomes section available at http://www.ncbi.nlm.nih.gov/genomes/static/euk_o.

The target DNA sequence described herein may comprise up to 1, 2, 3, 4, or 5 deletions, additions, mutations, and/or rearrangements as compared to a control sequence (naturally occurring sequence). In some embodiments, the mutations may be functional or nonfunctional. For example, a functional mutation may have an effect on a cellular function when the mutation is present in a host cell as compared to a control cell without the mutation. A non-functional mutation may be silent in function, for example, there is no discernable difference in phenotype of a host cell without the mutation as compared to a cell with the mutation.

Captured and/or modified large pieces of DNA (e.g., target DNA), may comprise a minimal or minimized chloroplast genome (e.g., the minimum number of genes and/or DNA fragment, required for chloroplast functionality). The captured and/or modified DNA may comprise the essential chloroplast genes, it may comprise a portion or all, or substantially all of the essential chloroplast genes. An essential gene may be a gene that is essential for one or more metabolic processes or biochemical pathways. An essential gene may be a gene required for chloroplast function, such as photosynthesis, carbon fixation, or hydrocarbon production. An essential gene may also be a gene that is essential for gene expression, such as transcription, translation, or other process(es) that affect gene expression. The essential genes may comprise mutations or rearrangements. Essential genes may also comprise a minimally functional set of genes to perform a function. For example, a particular function (e.g., photosynthesis) may be performed inefficiently by a set of genes/gene products, however, this set would still comprise essential genes because the function is still performed.

Modified DNA may comprise up to 5, 10, 15, 20, 25, 30, 40, or 50 essential genes. In some embodiments, the DNA may comprise essential chloroplast genomic sequence of up to 150 kb in length. The DNA may comprise essential chloroplast genes as well as non-essential chloroplast gene sequences. The DNA may be single stranded or double stranded, linear or circular, relaxed or supercoiled. The DNA may also be in the form of an expression cassette. For example, an expression cassette may comprise an essential gene to be expressed in a host cell. The expression cassette may comprise one or more essential genes as well as DNA sequences that promote the expression of the essential genes. The expression cassette may also comprise a region for integration into target DNA of a host. The expression cassette may also comprise one or more essential genes and one or more genes not naturally occurring in a host cell comprising the expression cassette. One of ordinary skill in the arts will easily ascertain various combinations of the aforementioned aspects of the expression cassettes.

In other instances, captured and/or modified pieces of DNA may comprise the entire genome of a plastid or organelle. For example, about 10%, 11%, 12%, 13%, 14%, 15%, 16%; 17%, 18%, 19%, 20%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of a plastid genome, or more. In one embodiment the captured and/or modified large pieces of DNA may comprise 10-100%, 20-100%, 30-100%, 40-100%, 50-100%, 60-100%, 70-100%, 80-100%, or 90-100% of the entire genome of a plastid or cell.

In still other instances, the captured and/or modified large pieces of DNA may comprise about 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb, 36 kb, 37 kb, 38 kb, 39 kb, 40 kb, 41 kb, 42 kb, 43 kb, 44 kb, 45 kb, 46 kb, 47 kb, 48 kb, 49 kb, 50 kb, 51 kb, 52 kb, 53 kb, 54 kb, 55 kb, 56 kb, 57 kb, 58 kb, 59 kb, 60 kb, 61 kb, 62 kb, 63 kb, 64 kb, 65 kb, 66 kb, 67 kb, 68 kb, 69 kb, 70 kb, 71 kb, 72 kb, 73 kb, 74 kb, 75 kb, 76 kb, 77 kb, 78 kb, 79 kb, 80 kb, 81 kb, 82 kb, 83 kb, 84 kb, 85 kb, 86 kb, 87 kb, 88 kb, 89 kb, 90 kb, 91 kb, 92 kb, 93 kb, 94 kb, 95 kb, 96 kb, 97 kb, 98 kb, 99 kb, 100 kb, 101 kb, 102 kb, 103 kb, 104 kb, 105 kb, 106 kb, 107 kb, 108 kb, 109 kb, 110 kb, 111 kb, 112 kb, 113 kb, 114 kb, 115 kb, 116 kb, 117 kb, 118 kb, 119 kb, 120 kb, 121 kb, 122 kb, 123 kb, 124 kb, 125 kb, 126 kb, 127 kb, 128 kb, 129 kb, 130 kb, 131 kb, 132 kb, 133 kb, 134 kb, 135 kb, 136 kb, 137 kb, 138 kb, 139 kb, 140 kb, 141 kb, 142 kb, 143 kb, 144 kb, 145 kb, 146 kb, 147 kb, 148 kb, 149 kb, 150 kb, 151 kb, 152 kb, 153 kb, 154 kb, 155 kb, 156 kb, 157 kb, 158 kb, 159 kb, 160 kb, 161 kb, 162 kb, 163 kb, 164 kb, 165 kb, 166 kb, 167 kb, 168 kb, 169 kb, 170 kb, 171 kb, 172 kb, 173 kb, 174 kb, 175 kb, 176 kb, 177 kb, 178 kb, 179 kb, 180 kb, 181 kb, 182 kb, 183 kb, 184 kb, 185 kb, 186 kb, 187 kb, 188 kb, 189 kb, 190 kb, 191 kb, 192 kb, 193 kb, 194 kb, 195 kb, 196 kb, 197 kb, 198 kb, 199 kb, 200 kb, 201 kb, 202 kb, 203 kb, 204 kb, 205 kb, 206 kb, 207 kb, 208 kb, 209 kb, 210 kb, 211 kb, 212 kb, 213 kb, 214 kb, 215 kb, 216 kb, 217 kb, 218 kb, 219 kb, 220 kb, 221 kb, 222 kb, 223 kb, 224 kb, 225 kb, 226 kb, 227 kb, 228 kb, 229 kb, 230 kb, 231 kb, 232 kb, 233 kb, 234 kb, 235 kb, 236 kb, 237 kb, 238 kb, 239 kb, 240 kb, 241 kb, 242 kb, 243 kb, 244 kb, 245 kb, 246 kb, 247 kb, 248 kb, 249 kb, 50 kb, 51 kb, 252 kb, 253 kb, 254 kb, 255 kb, 256 kb, 257 kb, 258 kb, 259 kb, 260 kb, 261 kb, 262 kb, 263 kb, 264 kb, 265 kb, 266 kb, 267 kb, 268 kb, 269 kb, 270 kb, 271 kb, 272 kb, 273 kb, 274 kb, 275 kb, 276 kb, 277 kb, 278 kb, 279 kb, 280 kb, 281 kb, 282 kb, 283 kb, 284 kb, 285 kb, 286 kb, 287 kb, 288 kb, 289 kb, 290 kb, 291 kb, 292 kb, 293 kb, 294 kb, 295 kb, 296 kb, 297 kb, 298 kb, 299 kb, 300 kb, 301 kb, 302 kb, 303 kb, 304 kb, 305 kb, 306 kb, 307 kb, 308 kb, 309 kb, 310 kb, 311 kb, 312 kb, 313 kb, 314 kb, 315 kb, 316 kb, 317 kb, 318 kb, 319 kb, 320 kb, 321 kb, 322 kb, 323 kb, 324 kb, 325 kb, 326 kb, 327 kb, 328 kb, 329 kb, 330 kb, 331 kb, 332 kb, 333 kb, 334 kb, 335 kb, 336 kb, 337 kb, 338 kb, 339 kb, 340 kb, 341 kb, 342 kb, 343 kb, 344 kb, 345 kb, 346 kb, 347 kb, 348 kb, 349 kb, 350 kb, 351 kb, 352 kb, 353 kb, 354 kb, 355 kb, 356 kb, 357 kb, 358 kb, 359 kb, 360 kb, 361 kb, 362 kb, 363 kb, 364 kb, 365 kb, 366 kb, 367 kb, 368 kb, 369 kb, 370 kb, 371 kb, 372 kb, 373 kb, 374 kb, 375 kb, 376 kb, 377 kb, 378 kb, 379 kb, 380 kb, 381 kb, 382 kb, 383 kb, 384 kb, 385 kb, 386 kb, 387 kb, 388 kb, 389 kb, 390 kb, 391 kb, 392 kb, 393 kb, 394 kb, 395 kb, 396 kb, 397 kb, 398 kb, 399 kb, 400 kb or more genomic (e.g., nuclear or organelle) DNA. In some embodiments the captured and or modified large pieces of DNA may comprise about 10-400 kb, 50-350 kb, 100-300 kb, 100-200 kb, 200-300 kb, 150-200 kb, 200-250 kb genomic DNA

In still other instances, the captured and or modified large pieces of DNA may comprise about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 50, 51, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300 or more open reading frames, partial open reading frames, pseudogenes and/or repeating sequences.

This disclosure also provides vectors comprising a cassette-able chloroplast genome or portion thereof (e.g., a removable DNA fragment comprising a chloroplast genome or functional portion thereof). A vector of the present disclosure may comprise functional chloroplast units (e.g., a unit essential for metabolic processes, photosynthesis, gene expression, photosystem I, photosystem II). Vectors of the present disclosure may comprise a transplantable chloroplast genome or portion thereof. Additionally, the vectors of the present disclosure may comprise a transferable chloroplast genome or portion thereof. In other embodiments, the vectors comprise: 1) one or more large pieces of modified DNA; 2) all genes necessary to carry out photosynthesis; 3) all genes required for chloroplast survival and/or function; 4) essential chloroplast genes; and/or 5) sufficient naturally occurring or modified chloroplast genes to perform one or more chloroplast functions, such as photosynthesis. A vector may comprise a portion, substantially all, or all of the essential chloroplast genes. A vector may comprise up to 5, 10, 15, 20, 25, 30, 40, 50, 60, 70, 80, 90, 100 or more essential genes.

A vector may comprise chloroplast DNA of 10, 20, 30, 40, 50, 60, 70, 80, 90, 100, 110, 120, 130, 140, 150, 160, 170, 180, 190, 200, 210, 220, 230, 240, 250 kb or more in length. A vector may comprise essential chloroplast genes as well as non-essential chloroplast gene sequences. A vector may comprise one or more, or all, essential chloroplast genes and/or one or more genes not naturally occurring in a host cell comprising a vector. In some embodiments, a vector may comprise chloroplast genes and genes not naturally occurring in the chloroplast. A vector may comprise one or more essential chloroplast genes and/or one or more DNA sequences or genes involved in chloroplast function, photosynthesis, carbon fixation, and/or hydrocarbon production. For example, a vector may comprise a sequence required for photosynthesis and a sequence involved in the isoprenoid production, MVA, and/or MEP pathways, such as a DNA sequence encoding a terpene synthase, or other polypeptide that produces a hydrocarbon, such as a terpene or isoprenoid. The disclosure further provides methods for cloning, manipulating and delivering a large target nucleic acid to a cell or particle, such as, for example, yeasts or bacteria. Certain embodiments of this method make use of a hybrid yeast-bacteria cloning system (See, e.g., U.S. Pat. Nos. 5,866,404 and 7,083,971 and Hokanson et al., (2003) Human Gene Ther.: 14: 329-339). The vectors herein (e.g., cloning system) is comprised of a shuttle vector that contains elements for function and replication in both yeast and bacteria, allowing it to stably function and replicate in either organism. This composition of functional and replicative elements yields a hybrid system which enjoys the benefits of both genetic engineering systems. The genetics of yeast (e.g., S. cerevisiae) are well understood and a powerful assortment of molecular biology tools exists for genetic engineering in yeast.

Another aspect produces a gap-filled vector by homologous recombination among the two arms and the target nucleic acid. In still another embodiment, at least one arm further comprises an origin of replication. In another embodiment, each arm further comprises a rare restriction endonuclease recognition site. Homologous recombination may be performed in vitro or in vivo, for example, in a fungal cell (e.g., S. cerevisiae, Sz. pombe or U. maydis). Also provided is a eukaryotic host cell harboring the recombinational cloning system or vector according to the disclosure, for example, in a fungal cell (e.g., S. cerevisiae, Sz. pombe or U. maydis).

A gap-filled linear vector may be converted to a circular vector in vitro (e.g. using T4 ligase) or in vivo, for example, in a bacterium. The circular vectors of interest can be amplified, purified, cut and used to recover sufficient amounts of DNA to be introduced either directly into a cell or into a suitable delivery system for subsequent delivery to a target cell. The methodology offers great versatility to clone and to modify any large bacterial or non-bacterial genome, and easily facilitate the use thereof as recombinational vectors. Direct delivery of a gap-filled vector into a cell may be performed by methods well known in the field such as, for example, calcium phosphate transformation methodologies or electroporation (see Sambrook et al., supra).

Accordingly, provided is a method for producing a recombinant delivery unit including the steps of: (a) producing a gap-filled vector containing a target sequence; (b) optionally circularizing the gap-filled vector segments; (c) propagating the vector; and (d) introducing the gap-filled vector in a delivery unit.

Bacterial systems are useful for amplifying and purifying DNA, and for functionally testing the genetic modifications and their effect on pathways. One embodiment provides cloning system will aid in the cloning and modifying of any large genome and easily facilitate the cloning and introduction of pathways. With the ability to deliver whole pathways, certain embodiments allow for a system biological approach to problem solving.

In general, target DNA (e.g., genomic DNA) may be captured by creating sites allowing for homologous recombination in the vector. For example, such sites may be created by, but not limited to, gap-repair cloning, wherein a gap is created in the vector, usually by restrictive enzyme digestion prior to transformation into the yeast. When the target DNA is mixed with the vector, the target DNA is recombined into the vector. This operation is called “gap filling.” This recombination can occur in bacteria, yeast, the original host organism, another organism, or in vitro. In some embodiments, recombination is performed in yeast because of the high rate of homologous recombination. Once captured, the target DNA can be modified in many ways including adding, altering, or removing DNA sequences. In some embodiments, the target DNA is genomic DNA. In other embodiments, the target DNA is organelle (e.g., mitochondria or chloroplast) DNA.

In some embodiments, target DNA is modified by adding, altering or removing genes, coding sequences, partial coding sequences, regulatory elements, positive and/or negative selection markers, recombination sites, restriction sites, and/or codon bias sites. For example, the target DNA sequence may be codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. The codons of the present disclosure may be A/T rich, for example, A/T rich in the third nucleotide position of the codons. Typically, the A/T rich codon bias is used for algae. In some embodiments, at least 50% of the third nucleotide position of the codons are A or T. In other embodiments, at least 60%, 70%, 80%, 90%, or 99% of the third nucleotide position of the codons are A or T. (see also U.S. Publication No. 2004/0014174).

Such manipulations are well known in the art and can be performed in numerous ways. In some embodiments, the modifications may be performed using cloned sequences. In other embodiments, the modifications may be performed using synthetic DNA.

Genetic manipulations include cloning large pieces of target DNA (e.g., chromosomes, genomes) and/or dividing and reorganizing target DNA based on functional relations between genes, such as metabolic pathways or operons. Genetic manipulations also include introducing and removing metabolic pathways, recombining DNA into functional units (e.g., metabolic pathways, synthetic operons), and/or determining sites of instability in large pieces of DNA (e.g., sites where a native or non-native host tends to delete or recombine a sequence of DNA).

Target DNA may be DNA from a prokaryote. Target DNA may also be genomic DNA, mitochondrial DNA, or chloroplast DNA from a eukaryote. Examples of such organisms from which genomic and/or organelle DNA may serve as target DNA include, but are not limited to Z. mobilis, algae (e.g., macroalgae or microalgae, such as Chlamydomonas reinhardtii), a rhodophyte, a chlorophyte, a heterokontophyte, a tribophyte, a glaucophyte, a chlorarachniophyte, a euglenoid, a haptophyte, a cryptomonad, a dinoflagellum, or a phytoplankton. One of skill in the art will recognize that these organisms are listed only as examples and that the methods disclosed herein are applicable to the large DNA from any organism, including bacteria, plants, fungi, protists, and animals. Genetic manipulations of the present disclosure may include stabilizing large pieces of DNA by removing or inserting sequences that force transformed cells to preserve certain sequences of DNA and to stably maintain the sequences in its progeny. Genetic manipulations may also include altering codons of the target DNA, vector DNA, and/or synthetic DNA to reflect any codon bias of the host organism. Additionally, genetic manipulations of the present disclosure may include determining the minimal set of genes required for an organism to be viable. In another embodiment, the genetic manipulations of the present disclosure include determining the minimal set of genes required for a certain metabolic pathway to be created or maintained.

The genetic manipulations may include determining redundant genes both within a genome, and between two genomes (e.g., redundancy between the nuclear and chloroplast genome). Additionally, the genetic manipulations may include determining a functional sequence of DNA that could be artificially synthesized (e.g. the genes in a certain metabolic pathway, the genes of a functional genome). In another embodiment, the genetic manipulations of the present disclosure include creating DNA and genomes packaged into cassettes (e.g., sites within a vector where genes can be easily inserted or removed). The genetic manipulations of the present disclosure may also include creating a nuclear or organelle genome that is viable in multiple species (e.g. a transplantable chloroplast genome). Furthermore, the genetic manipulations may include a method for testing the viability of any of these manipulations or creations (e.g., transferring a shuttle vector back into a host system and assaying for survival).

Vectors, Markers and Transformation

A vector or other recombinant nucleic acid molecule may include a nucleotide sequence encoding a selectable marker. The term or “selectable marker” or “selection marker” refers to a polynucleotide (or encoded polypeptide) that confers a detectable phenotype. A selectable marker generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (Giacomin, Plant Sci. 116:59-72, 1996; Scikantha, J. Bacteriol. 178:121, 1996; Gerdes, FEBS Lett. 389:44-47, 1996; see, also, Jefferson, EMBO J. 6:3901-3907, 1997, fl-glucuronidase). A selectable marker generally is a molecule that, when present or expressed in a cell, provides a selective advantage (or disadvantage) to the cell containing the marker, for example, the ability to grow in the presence of an agent that otherwise would kill the cell.

A selectable marker can provide a means to obtain prokaryotic cells or plant cells or both that express the marker and, therefore, can be useful as a component of a vector of the disclosure (see, for example, Bock, supra, 2001). Examples of selectable markers include, but are not limited to, those that confer antimetabolite resistance, for example, dihydrofolate reductase, which confers resistance to methotrexate (Reiss, Plant Physiol. (Life Sci. Adv.) 13:143-149, 1994); neomycin phosphotransferase, which confers resistance to the aminoglycosides neomycin, kanamycin and paromycin (Herrera-Estrella, EMBO J. 2:987-995, 1983), hygro, which confers resistance to hygromycin (Marsh, Gene 32:481-485, 1984), trpB, which allows cells to utilize indole in place of tryptophan; hisD, which allows cells to utilize histinol in place of histidine (Hartman, Proc. Natl. Acad. Sci., USA 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (White et al., Nucl. Acids Res. 18:1062, 1990; Spencer et al., Theor. Appl. Genet. 79:625-631, 1990), a mutant EPSPV-synthase, which confers glyphosate resistance (Hinchee et al., BioTechnology 91:915-922, 1998), a mutant acetolactate synthase, which confers imidazolione or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells and tetracycline; ampicillin resistance for prokaryotes such as E. coli; and bleomycin, gentamycin, glyphosate, hygromycin, kanamycin, methotrexate, phleomycin, phosphinotricin, spectinomycin, streptomycin, sulfonamide and sulfonylurea resistance in plants (see, for example, Maliga et al., Methods in Plant Molecular Biology, Cold Spring Harbor Laboratory Press, 1995, page 39).

Methods for nuclear and plastid transformation are routine and well known for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

Microprojectile mediated transformation also can be used to introduce a polynucleotide into a plant cell chloroplast (Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known in the art (see, e.g.; Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (Duan et al., Nature Biotech. 14:494-498, 1996; Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, the glass bead agitation method, and the like.

Transformation frequency may be increased by replacement of recessive rRNA or r-protein antibiotic resistance genes with a dominant selectable marker, including, but not limited to the bacterial aadA gene (Svab and Maliga, Proc. Natl. Acad. Sci., USA 90:913-917, 1993). Approximately 15 to 20 cell division cycles following transformation are generally required to reach a homoplastidic state. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein.

Any of the nucleotide sequences of target DNA, vector DNA, or synthetic DNA on the vectors can further include codons biased for expression of the nucleotide sequences in the organism transformed. In some instances, codons in the nucleotide sequences are A/T rich in a third nucleotide position of the codons. For example, at least 50% of the third nucleotide position of the codons may be A or T. In other instances, the codons are G/C rich, for example at least 50% of the third nucleotide positions of the codons may be G or C.

The nucleotide sequences of the shuttle vectors of the present disclosure can be adapted for chloroplast expression. For example, a nucleotide sequence herein can comprise a chloroplast specific promoter or chloroplast specific regulatory control region. The nucleotide sequences can also be adapted for nuclear expression. For example, a nucleotide sequence can comprise a nuclear specific promoter or nuclear specific regulatory control regions. The nuclear sequences can encode a protein with a targeting sequence that encodes a chloroplast targeting protein (e.g., a chloroplast transit peptide), or a signal peptide that directs a protein to the endomembrane system for deposition in the endoplasmic reticulum or plasma membrane.

In embodiments where a vector encodes genes capable of fuel production, fuel products are produced by altering the enzymatic content of the cell to increase the biosynthesis of specific fuel molecules. For example, nucleotides sequences (e.g., an ORF isolated from an exogenous source) encoding biosynthetic enzymes can be introduced into the chloroplast of a photosynthetic organism. Nucleotide sequences encoding fuel biosynthetic enzymes can also be introduced into the nuclear genome of the photosynthetic organisms. Nucleotide sequences introduced into the nuclear genome can direct accumulation of the biosynthetic enzyme in the cytoplasm of the cell, or may direct accumulation of the biosynthetic enzyme in the chloroplast of the photosynthetic organism.

Any of the nucleotide sequences herein may further comprise a regulatory control sequence. Regulatory control sequences can include one or more of the following: a promoter, an intron, an exon, processing elements, 3′ untranslated region, 5′ untranslated region, RNA stability elements, or translational enhancers A promoter may be one or more of the following: a promoter adapted for expression in the organism, an algal promoter, a chloroplast promoter, and a nuclear promoter, any of which may be a native or synthetic promoters. A regulatory control sequence can be inducible or autoregulatable. A regulatory control sequence can include autologous and/or heterologous sequences. In some cases, control sequences can be flanked by a first homologous sequence and a second homologous sequence. The first and second homologous sequences can each be at least 500 nucleotides in length. The homologous sequences can allow for either homologous recombination or can act to insulate the heterologous sequence to facilitate gene expression.

Vectors may also comprise sequences involved in producing products useful as biopharmaceuticals, such as, but not limited to, antibodies (including functional portions thereof), interleukins and other immune modulators, and antibiotics. See, e.g., Mayfield et al., (2003) Proc. Nat'l Acad. Sci.: 100 (43842) and U.S. Pub. No. 2004/0014174.

Vectors of the present disclosure may comprise a cassette-able bacterial genome or portion thereof (e.g., a removable DNA fragment comprising a bacterial genome or functional portion thereof). Additionally, may comprise functional genomic units (e.g., a unit essential for metabolic processes, biochemical pathways, gene expression). Vectors disclosed herein may comprise a transplantable bacterial genome or portion thereof. Vectors may comprise a transferable bacterial genome or portion thereof.

In some embodiments, the large piece of target DNA is modified. The modified DNA may comprise all genes necessary to carry out ethanologenesis, all genes required for the Entner-Duodorff pathway, the glucose tolerance pathway, the ethanol tolerance pathway, the carboxylic acid byproduct resistance pathway, the acetic acid tolerance pathway, the sugar transport pathway, sugar fermentation pathways, and/or the cellulose and hemicellulose digestive pathways.

Hybrid cloning systems and methods disclosed herein combine the high versatility of yeast as a system for the capture and manipulation of a given nucleic acid and the high efficiency of bacterial systems for the amplification of such nucleic acid. Recombinational vectors relying on homologous recombination to mediate the isolation, manipulation and delivery of large nucleic acid fragments were constructed. Described herein are methods for using such recombinational cloning vectors to clone, to manipulate and to deliver large nucleic acids. Finally, methods are provided for using such recombinational cloning systems as potentiators of biochemical pathway analysis, organelle analysis, and synthetic chloroplast construction.

The vectors disclosed herein may be introduced into yeast. The yeast may be a suitable strain of Saccharomyces cerevisiae; however, other yeast models may be utilized. Introduction of vectors into yeast may allow for genetic manipulation of the vectors. Yeast vectors have been described extensively in the literature and methods of manipulating the same also are well known as discussed hereinafter (see e.g., Ketner et al. (1994) Proc. Natl. Acad. Sci. (USA) 91:6186 6190).

Following genetic manipulation, the cloning system may allow for the transition to a bacterial environment, suitable for the preparation of larger quantities of nucleic acids. Representative examples of a bacterial type vectors include, but are not limited to, the P1 artificial chromosome, bacterial artificial chromosome (BAC) and single copy plasmid F factors (Shizuya et al. (1992) Proc. Natl. Acad. Sci. 89:8794 8797). Similarly, bacterial vectors are well known in the art (e.g., Ioannou et al. (1994) Nature 6:84 89). The disclosure also provides a shuttle vector comprising a yeast selectable marker, a bacterial selectable marker, a telomere, a centromere, a yeast origin of replication, and/or a bacterial origin of replication.

Shuttle vectors of the present disclosure may enable homologous recombination in yeast to capture and to integrate in a vector of interest a target nucleic acid of interest. Shuttle vectors may allow for the manipulation of target DNA in any of the hosts to which the vectors can be introduced. In some embodiments, after desired manipulations, shuttle vector components may be removed, leaving just the modified target DNA. Such extraction of vector sequences can be performed using standard methodologies and may occur in any host cell. The target nucleic acid of interest can be a large nucleic acid, and can include, for example, a vector, such as a viral vector, including the foreign gene of interest contained therein. The target nucleic acid can also be a bacterial (including archaebacteria and eubacteria), viral, fungal, protist, plant or animal genome, or a portion thereof. For example, the target nucleic acid of one embodiment of the present disclosure comprises an entire prokaryotic genome. As an additional example, a target nucleic acid of the present disclosure may comprises the chloroplast genome of a eukaryotic organism.

Shuttle vectors according to the present disclosure may comprise an appropriately oriented DNA that functions as a telomere in yeast and a centromere. Any suitable telomere may be used. Suitable telomeres include without limitation telomeric repeats from many organisms, which can provide telomeric function in yeast. The terminal repeat sequence in humans (TTAGGG)_{N}, is identical to that in trypanosomes and similar to that in yeast ((TG)₁₋₃)_{N} and Tetrahymena (TTGGG)_{N} (Szostak & Blackburn (1982) Cell 29:245 255; Brown (1988) EMBO J. 7:2377 2385; and Moyzis et al. (1988) Proc. Natl. Acad. Sci. 85:6622 6626).

The term “centromere” is used herein to identify a nucleic acid, which mediates the stable replication and precise partitioning of the vectors of the disclosure at meiosis and at mitosis thereby ensuring proper segregation into daughter cells. Suitable centromeres include, without limitation, the yeast centromere, CEN4, which confers mitotic and meiotic stability on large linear plasmids (Murray & Szostak (1983) Nature 305:189 193; Carbon (1984) Cell 37:351 353; and Clark et al. (1990) Nature 287:504 509)).

In some embodiments, at least one of the two segments of the circular vector according to the disclosure includes at least one replication system that is functional in a host cell/particle of choice. As it will become apparent hereinafter, one of skill will realize that the manipulation, amplification and/or delivery of a target nucleic acid of choice may entail the use of more than one host cell/particle. Accordingly, more than one replication system functional in each host cell/particle of choice may be included.

When a host cell(s) is a prokaryote, particularly E. coli, replication system(s) include those which are functional in prokaryotes, such as, for example, P1 plasmid replicon, ori, P1 lytic replicon, ColE1, BAC, single copy plasmid F factors and the like. Either one or both segments, and/or the circular vector, may further include a yeast origin of replication capable of supporting the replication of large nucleic acids. Non-limiting examples of replication regions include the autonomously replicating sequence or “ARS element.” ARS elements were identified as yeast sequences that conferred high-frequency transformation. Tetrahymena DNA termini have been used as ARS elements in yeast along with ARS1 and ARS4 (Kiss et al. (1981) Mol. Cell. Biol. 1:535 543; Stinchcomb et al. (1979) Nature 282:39; and Barton & Smith (1986) Mol. Cell. Biol. 6:2354). For each segment (e.g., those corresponding to the yeast and bacterial elements of the gap-filling shuttle vector) there may be two or more origins of replication.

The first and/or the second segment according to one aspect may be joined in a circularized vector form (e.g., plasmid form). Circularization can occur in vivo or in vitro using the segment of interest. Alternatively, a circular vector of interest can be used. As used herein, the term “vector” designates a plasmid or phage DNA or other nucleic acid into which DNA or other nucleic acid may be cloned. The vector may replicate autonomously in a host cell and may be characterized further by one or a small number of restriction endonuclease recognition sites at which such nucleic acids may be cut in a determinable fashion and into which nucleic acid fragments may be inserted. The vector further may contain a selectable marker suitable for the identification of cells transformed with the vector.

Target nucleic acids may vary considerably in complexity. The target nucleic acid may include viral, prokaryotic or eukaryotic DNA, cDNA, exonic (coding), and/or intronic (noncoding) sequences. Hence, the target nucleic acid may include one or more genes. A target nucleic acid may be a chromosome, genome, or operon and/or a portion of a chromosome, genome or operon. A target nucleic acid may comprise coding sequences for all the genes in a pathway, the minimum complement of genes necessary for survival of an organelle, and/or the minimum complement of genes necessary for survival of an organism. A target nucleic acid may comprise Zymomonas mobilis DNA sequence, including, but not limited to genomic DNA and/or cDNA. A target nucleic acid may comprise eukaryotic chloroplast DNA sequence, including but not limited to, chloroplast genome DNA and/or cDNA. A target nucleic acid may comprise cyanobacteria DNA, including but not limited to genomic DNA and/or cDNA. The target nucleic acid also may be of any origin and/or nature.

It may be desirable for the gene to also comprise a promoter operably linked to the coding sequence in order to effectively promote transcription. Enhancers, repressors and other regulatory sequences may also be included in order to modulate activity of the gene, as is well known in the art. A gene as provided herein can refer to a gene that is found in the genome of the individual host cell (i.e., endogenous) or to a gene that is not found in the genome of the individual host cell (i.e., exogenous or a “foreign gene”). Foreign genes may be from the same species as the host or from different species. For transfection of a cell using DNA containing a gene with the intent that the gene will be expressed in the cell, the DNA may contain any control sequences necessary for expression of the gene in the required orientation for expression. The term “intron” as used herein, refers to a DNA sequence present in a given gene which is not translated into protein and is generally found between exons.

Genetic elements, or polynucleotides comprising a region that encodes a polypeptide or a region that regulates transcription or translation or other processes important to expression of the polypeptide in a host cell, or a polynucleotide comprising both a region that encodes a polypeptide and a region operably linked thereto that regulates expression. Genetic elements may be comprised within a vector that replicates as an episomal element; that is, as a molecule physically independent of the host cell genome. They may be comprised within mini-chromosomes, such as those that arise during amplification of transfected DNA by methotrexate selection in eukaryotic cells. Genetic elements also may be comprised within a host cell genome; not in their natural state but, rather, following manipulation such as isolation, cloning and introduction into a host cell in the form of purified DNA or in a vector, among others.

Vectors of the present disclosure may contain sufficient linear identity or similarity (homology) to have the ability to hybridize to a portion of a target nucleic acid made or which is single-stranded, such as a gene, a transcriptional control element or intragenic DNA. Without being bound to theory, such hybridization is ordinarily the result of base-specific hydrogen bonding between complementary strands, preferably to form Watson-Crick base pairs. As a practical matter, such homology can be inferred from the observation of a homologous recombination event. In some embodiments, such homology is from about 8 to about 1000 bases of the linear nucleic acid. In other embodiments, such homology is from about 12 to about 500 bases. One skilled in the art will appreciate that homology may extend over longer stretches of nucleic acids.

Homologous recombination is a type of genetic recombination, a process of physical rearrangement occurring between two strands of DNA. Homologous recombination involves the alignment of similar sequences, a crossover between the aligned DNA strands, and breaking and repair of the DNA to produce an exchange of material between the strands. The process homologous recombination naturally occurs in organisms and is also utilized as a molecular biology technique for introducing genetic changes into organism.

The vectors described herein may be modified further to include functional entities other than the target sequence which may find use in the preparation of the construct(s), amplification, transformation or transfection of a host cell, and—if applicable—for integration in a host cell. For example, the vector may comprise regions for integration into host DNA. Integration may be into nuclear DNA of a host cell. In some embodiments, integration may be into non-nuclear DNA, such as chloroplast DNA. Other functional entities of the vectors may include, but are not limited to, markers, linkers and restriction sites.

A target nucleic acid may include a regulatory nucleic acid. This refers to any sequence or nucleic acid which modulates (either directly or indirectly, and either up or down) the replication, transcription and/or expression of a nucleic acid controlled thereby. Control by such regulatory nucleic acid may make a nucleic acid constitutively or inducibly transcribed and/or translated. Any of the nucleotide sequences herein may further comprise a regulatory control sequence. Examples of regulatory control sequences can include, without limitation, one or more of the following: a promoter, an intron, an exon, processing elements, 3′ untranslated region, 5′ untranslated region, RNA stability elements, or translational enhancers. A promoter may be one or more of the following: a promoter adapted for expression in the organism (e.g., bacterial, fungal, viral, plant, mammalian, or protist), an algal promoter, a chloroplast (or other plastid) promoter, a mitochondrial promoter, and a nuclear promoter, any of which may be a native or synthetic promoters. A regulatory control sequence can be inducible or autoregulatable. A regulatory control sequence can include autologous and/or heterologous sequences. In some cases, control sequences can be flanked by a first homologous sequence and a second homologous sequence. The first and second homologous sequences can each be at least 500 nucleotides in length. The homologous sequences can allow for either homologous recombination or can act to insulate the heterologous sequence to facilitate gene expression.

In some instances, target DNA, vector DNA or other DNA present in a shuttle vector does not result in production of a polypeptide product but rather allows for secretion of the product from the cell. In these cases, the nucleotide sequence may encode a protein that enhances or initiates or increases the rate of secretion of a product from an organism to the external environment. Thus, segments of vectors and/or vectors of the disclosure may include a transcriptional regulatory region such as, for example, a transcriptional initiation region. One skilled in the art will appreciate that a multitude of transcriptional initiation sequences have been isolated and are available, including thymidine kinase promoters, beta-actin promoters, immunoglobin promoters, methallothionein promoters, human cytomegalovirus promoters and SV40 promoters.

One embodiment provides a method of producing a gap-filled vector. A gap-filled vector may undergo homologous recombination and insertion of a target nucleic acid according to the disclosure by filling in the region (gap) between the sequences homologous to the 5′ and the 3′ regions of the target nucleic acid. Hence, in some embodiments, one would contact the instant cloning system with a target nucleic acid under conditions that allow homologous recombination.

Another method combines: (i) a first segment including a first nucleic acid homologous to the 5′ terminus of a target nucleic acid, a first selectable marker and a first cyclization element; (ii) a target nucleic acid; and (iii) a second segment including a second nucleic acid homologous to the 3′ terminus of a target nucleic acid, a second selectable marker and a second cyclization element, under conditions which allow homologous recombination. One embodiment of the disclosure produces a gap-filled vector by homologous recombination between the two arms and the target nucleic acid. The exchange between the homologous regions found in the arms and the target nucleic acid is effected by homologous recombination at any point between the homologous nucleic acids. With respect to a circular vector of the present disclosure, the “gap filling” essentially is insertion (i.e., subcloning) of the target sequence into the vector.

Homologous recombination may be effected in vitro according to methodologies well known in the art. For example, the method of the disclosure can be practiced using yeast lysate preparations. Homologous recombination may take place in vivo. Hence, the methods disclosed herein may be practiced using any host cell capable of supporting homologous recombination events such as, for example, bacteria, yeast and mammalian cells. One skilled in the art will appreciate that the choice of a suitable host depends on the particular combination of selectable markers used in the cloning system of the method.

Techniques that may be used to introduce the vector into a host cell of interest include calcium phosphate/DNA coprecipitation, electroporation, bacterial-protoplast fusion, microinjection of DNA into the nucleus and so on. One of skill will appreciate that a number of protocols may be used virtually interchangeably, for example, to transfect mammalian cells, as set forth for example in Keown et al. (Meth. Enzymol. 185:527 537, 1990).

Transformation may be achieved by using a soil bacterium, such as Agrobacterium tumefaciens. Agrobacterium tumefaciens may carry an engineered plasmid vector, or carrier of selected extra genes. Plant tissue, such as leaves, are cut in small pieces, eg. 10×10 mm, and soaked for 10 minutes in a fluid containing suspended Agrobacterium. Some cells along the cut are transformed by the bacterium, that inserts its DNA into the cell. Placed on selectable rooting and shooting media, the plants will regrow. Some plants species can be transformed just by dipping the flowers into suspension of Agrobacteria and then planting the seeds in a selective medium.

Another methodology is use of a “gene gun” approach. The gene gun is part of a method called the biolistic (also known as bioballistic) method, and under certain conditions, DNA (or RNA) become “sticky,” adhering to biologically inert particles such as metal atoms (usually tungsten or gold). By accelerating this DNA-particle complex in a partial vacuum and placing the target tissue within the acceleration path, DNA is effectively introduced. Uncoated metal particles could also be shot through a solution containing DNA surrounding the cell thus picking up the genetic material and proceeding into the living cell. A perforated plate stops the shell cartridge but allows the slivers of metal to pass through and into the living cells on the other side. The cells that take up the desired DNA, identified through the use of a marker gene (in plants the use of GUS is most common), are then cultured to replicate the gene and possibly cloned. The biolistic method is most useful for inserting genes into plant cells such as pesticide or herbicide resistance. Different methods have been used to accelerate the particles: these include pneumatic devices; instruments utilizing a mechanical impulse or macroprojectile; centripetal, magnetic or electrostatic forces; spray or vaccination guns; and apparatus based on acceleration by shock wave, such as electric discharge (for example, see Christou and McCabe, 1992, Agracetus, Inc. Particle Gun Transformation of Crop Plants Using Electric Discharge (ACCELL™ Technology)).

Transformation can be performed, for example, according to the method of Cohen et al. (Proc. Natl. Acad. Sci. USA, 69:2110 (1972)), the protoplast method (Mol. Gen. Genet., 168:111 (1979)), or the competent method (J. Mol. Biol., 56:209 (1971)) when the hosts are bacteria (E. coli, Bacillus subtilis, and such), the method of Hinnen et al. (Proc. Natl. Acad. Sci. USA, 75:1927 (1978)), or the lithium method (J. Bacteriol., 153:163 (1983)) when the host is S. cerevisiae, the method of Graham (Virology, 52:456 (1973)) when the hosts are animal cells, and the method of Summers et al. (Mol. Cell. Biol., 3:2156-2165 (1983)) when the hosts are insect cells. Typically, following a transformation event, potential transformants are plated on nutrient media for selection and/or cultivation.

The nutrient media preferably comprises a carbon source, an inorganic nitrogen source, or an organic nitrogen source necessary for the growth of host cells (transformants). Examples of the carbon source are glucose, dextran, soluble starch, and sucrose, and examples of the inorganic or organic nitrogen source are ammonium salts, nitrates, amino acids, corn steep liquor, peptone, casein, meat extract, soy bean cake, and potato extract. If desired, the media may comprise other nutrients (for example, an inorganic salt (for example, calcium chloride, sodium dihydrogenphosphate, and magnesium chloride), vitamins, antibiotics (for example, tetracycline, neomycin, ampicillin, kanamycin, etc.). Media for some photosynthetic organisms may not require a carbon source as such organisms may be photoautotrophs and, thus, can produce their own carbon sources.

Cultivation and/or selection are performed by methods known in the art. Cultivation and selection conditions such as temperature, pH of the media, and cultivation time are selected appropriately for the vectors, host cells and methods of the present disclosure. One of skill in the art will recognize that there are numerous specific media and cultivation/selection conditions which can be used depending on the type of host cell (transformant) and the nature of the vector (e.g., which selectable markers are present). The media herein are merely described by way of example and are not limiting.

When the hosts are bacteria, actinomycetes, yeast, or filamentous fingi, media comprising the nutrient source(s) mentioned above are appropriate. When the host is E. coli, examples of useful media are LB media, M9 media (Miller et al. Exp. Mol. Genet., Cold Spring Harbor Laboratory, p. 431 (1972)), and so on. When the host is yeast, an example of medium is Burkhoter minimal medium (Bostian, Proc. Natl. Acad. Sci. USA, 77:4505 (1980)).

The selection of vectors in yeast may be accomplished by the use of yeast selectable markers. Examples include, but are not limited to, HIS3, TRP1, URA3, LEU2 and ADE markers. In some embodiments, a vector or segment thereof may comprise two or more selectable markers. Thus, in one embodiment, a segment of a vector may comprise an ADE marker to be lost upon homologous recombination with the target nucleic acid, and a HIS3 marker. The other segment may comprise a TRP1 marker. Selection is achieved by growing transformed cells on a suitable drop-out selection media (see e.g., Watson et al. (1992) Recombinant DNA, 2.sup.nd ed., Freeman and Co., New York, N.Y.). For example, HIS3 allows for selection of cells containing the first segment. TRP1 allows for selection of cells containing the second segment. ADE allows screening and selection of clones in which homologous recombination took place. ADE enables color selection (red).

Recombinant yeast cells may be selected using the selectable markers described herein according to methods well known in the art. Hence, one skilled in the art will appreciate that recombinant yeast cells harboring a gap-filled vector of the disclosure may be selected on the basis of the selectable markers included therein. For example, recombinant vectors carrying HIS3 and TRP1 may be selected by growing transformed yeast cells in the presence of drop-out selection media lacking histidine and tryptophan. Isolated positive clones may be purified further and analyzed to ascertain the presence and structure of the recombinant vector of the disclosure by, e.g., restriction analysis, electrophoresis, Southern blot analysis, polymerase chain reaction or the like. The disclosure further provides gap-filled vectors engineered according to the method of the disclosure. Such a vector is the product of homologous recombination between the segments or vectors of the disclosure and a target nucleic acid of choice. The disclosure also provides a prokaryotic cell and/or a eukaryotic host cell harboring the cloning system or vector disclosed herein. The organism can be unicellular or multicellular. The organism may be naturally photosynthetic or naturally non-photosynthetic. Other examples of organisms that can be transformed include vascular and non-vascular organisms. When hosts, such as plant, yeast, animal, algal, or insect cells are used, a vector of the present invention may contain, at least, a promoter, an initiation codon, the polynucleotide encoding a protein, and a termination codon. The vectors of the present invention may also contain, if required, a polynucleotide for gene amplification (marker) that is usually used.

Products

The vectors of the present invention may comprise sequences that result in production of a product naturally, or not naturally, produced in the organism comprising the vector. In some instances the product encoded by one or more sequences on a vector is a polypeptide, for example an enzyme. Enzymes utilized in practicing the present invention may be encoded by nucleotide sequences derived from any organism, including bacteria, plants, fungi and animals. Vectors may also comprise nucleotide sequences that affect the production or secretion of a product from the organism. In some instances, such nucleotide sequence(s) encode one or more enzymes that function in isoprenoid biosynthetic pathway. Examples of polypeptides in the isoprenoid biosynthetic pathway include synthases such as C5, C10, C15, C20, C30, and C40 synthases. In some instances, the enzymes are isoprenoid producing enzymes. In some instances, an isoprenoid producing enzyme produces isoprenoids with two phosphate groups (e.g., GPP synthase, FPP synthase, DMAPP synthase). In other instances, isoprenoid producing enzymes produce isoprenoids with zero, one, three or more phosphates or may produce isoprenoids with other functional groups. Polynucleotides encoding enzymes and other proteins useful in the present invention may be isolated and/or synthesized by any means known in the art, including, but not limited to cloning, sub-cloning, and PCR.

An isoprenoid producing enzyme for use in the present invention may also be botryococcene synthase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristolchene synthase, α-humulene, (E,E)-α-farnesene synthase, (−)-β-pinene synthase, γ-terpinene synthase, limonene cyclase, linalool synthase, (+)-bornyl diphosphate synthase, levopimaradiene synthase, isopimaradiene synthase, (E)-γ-bisabolene synthase, copalyl pyrophosphate synthase, kaurene synthase, longifolene synthase, γ-humulene synthase, δ-selinene synthase, β-phellandrene synthase, terpinolene synthase, (+)-3-carene synthase; syn-copalyl diphosphate synthase, α-terpineol synthase, syn-pimara-7,15-diene synthase, ent-sandaaracopimaradiene synthase, sterner-13-ene synthase, E-β-ocimene, S-linalool synthase, geraniol synthase, γ-terpinene synthase, linalool synthase, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, or manool synthase.

Other enzymes which may be produced by vectors of the present invention include biomass-degrading enzymes. Non-limiting examples of biomass-degrading enzymes include: cellulolytic enzymes, hemicellulolytic enzymes, pectinolytic enzymes, xylanases, ligninolytic enzymes, cellulases, cellobiases, softening enzymes (e.g., endopolygalacturonase), amylases, lipases, proteases, RNAses, DNAses, inulinase, lysing enzymes, phospholipases, pectinase, pullulanase, glucose isomerase, endoxylanase, beta-xylosidase, alpha-L-arabinofuranosidase, alpha-glucoronidase, alpha-galactosidase, acetylxylan esterase, and feruloyl esterase. Examples of genes that encode such enzymes include, but are not limited to, amylases, cellulases, hemicellulases, (e.g., β-glucosidase, endocellulase, exocellulase), exo-β-glucanase, endo-β-glucanase and xylanse (endoxylanase and exoxylanse). Examples of ligninolytic enzymes include, but are not limited to, lignin peroxidase and manganese peroxidase from Phanerochaete chryososporium. One of skill in the art will recognize that these enzymes are only a partial list of enzymes which could be used in the present invention.

The present invention contemplates making enzymes that contribute to the production of fatty acids, lipids or oils by transforming host cells (e.g., alga cells such as C. reinhardtii, D. salina, H. pluvalis and cyanobacterial cells) and/or organisms comprising host cells with nucleic acids encoding one or more different enzymes. In some embodiments the enzymes that contribute to the production of fatty acids, lipids or oils are anabolic enzymes. Some examples of anabolic enzymes that contribute to the synthesis of fatty acids include, but are not limited to, acetyl-CoA carboxylase, ketoreductase, thioesterase, malonyltransferase, dehydratase, acyl-CoA ligase, ketoacylsynthase, enoylreductase and a desaturase. In some embodiments the enzymes are catabolic or biodegrading enzymes. In some embodiments, a single enzyme is produced.

Some host cells may be transformed with multiple genes encoding one or more enzymes. For example, a single transformed cell may contain exogenous nucleic acids encoding enzymes that make up an entire fatty acid synthesis pathway. One example of a pathway might include genes encoding an acetyl CoA carboxylase, a malonyltransferase, a ketoacylsynthase, and a thioesterase. Cells transformed with entire pathways and/or enzymes extracted from them, can synthesize complete fatty acids or intermediates of the fatty acid synthesis pathway. In some embodiments constructs may contain multiple copies of the same gene, and/or multiple genes encoding the same enzyme from different organisms, and/or multiple genes with mutations in one or more parts of the coding sequences.

In some instances, a product (e.g. fuel, fragrance, insecticide) is a hydrocarbon-rich molecule, e.g. a terpene. A terpene (classified by the number of isoprene units) can be a hemiterpene, monoterpene, sesquiterpene, diterpene, triterpene, or tetraterpene. In specific embodiments the terpene is a terpenoid (aka isoprenoid), such as a steroid or carotenoid. Subclasses of carotenoids include carotenes and xanthophylls. In specific embodiments, a fuel product is limonene, 1,8-cineole, α-pinene, camphene, (+)-sabinene, myrcene, abietadiene, taxadiene, farnesyl pyrophosphate, amorphadiene, (E)-α-bisabolene, beta carotene, alpha carotene, lycopene, fusicoccadiene or diapophytoene. Some of these terpenes are pure hydrocarbons (e.g. limonene) and others are hydrocarbon derivatives (e.g. cineole).

Examples of fuel products include petrochemical products and their precursors and all other substances that may be useful in the petrochemical industry. Fuel products include, for example, petroleum products, and precursors of petroleum, as well as petrochemicals and precursors thereof. The fuel product may be used for generating substances, or materials, useful in the petrochemical industry, including petroleum products and petrochemicals. The fuel or fuel products may be used in a combustor such as a boiler, kiln, dryer or furnace. Other examples of combustors are internal combustion engines such as vehicle engines or generators, including gasoline engines, diesel engines, jet engines, and others. Fuel products may also be used to produce plastics, resins, fibers, elastomers, lubricants, and gels.

Examples of products contemplated herein include hydrocarbon products and hydrocarbon derivative products. A hydrocarbon product is one that consists of only hydrogen molecules and carbon molecules. A hydrocarbon derivative product is a hydrocarbon product with one or more heteroatoms, wherein the heteroatom is any atom that is not hydrogen or carbon. Examples of heteroatoms include, but not limited to, nitrogen, oxygen, sulfur, and phosphorus. Some products are hydrocarbon-rich, wherein as least 50%, 60%, 70%, 80%, 90%, or 95% of the product by weight is made up carbon and hydrogen.

Fuel products, such as hydrocarbons, may be precursors or products conventionally derived from crude oil, or petroleum, such as, but not limited to, liquid petroleum gas, naptha (ligroin), gasoline, kerosene, diesel, lubricating oil, heavy gas, coke, asphalt, tar, and waxes. For example, fuel products may include small alkanes (for example, 1 to approximately 4 carbons) such as methane, ethane, propane, or butane, which may be used for heating (such as in cooking) or making plastics. Fuel products may also include molecules with a carbon backbone of approximately 5 to approximately 9 carbon atoms, such as naptha or ligroin, or their precursors. Other fuel products may be about 5 to about 12 carbon atoms or cycloalkanes used as gasoline or motor fuel. Molecules and aromatics of approximately 10 to approximately 18 carbons, such as kerosene, or its precursors, may also be fuel products. Fuel products may also include molecules, or their precursors, with more than 12 carbons, such as used for lubricating oil. Other fuel products include heavy gas or fuel oil, or their precursors, typically containing alkanes, cycloalkanes, and aromatics of approximately 20 to approximately 70 carbons. Fuel products also includes other residuals from crude oil, such as coke, asphalt, tar, and waxes, generally containing multiple rings with about 70 or more carbons, and their precursors.

Host Cells and Organisms

Examples of organisms that can be transformed using the vectors and methods herein include vascular and non-vascular organisms. The organism can be prokaroytic or eukaroytic. The organism can be unicellular or multicellular.

Eukaryotic cells, such as a fungal cell (e.g., Saccharomyces cerevisiae, Schizosaccharomyces pombe or Ustilago maydis) may be transformed using the methods and compositions of the present invention. Methods for introducing nucleic acids in a fungal/yeast cells are well known in the art. Hence, such a step may be accomplished by conventional transformation methodologies. Non-limiting examples of suitable methodologies include electroporation, alkali cations protocols and spheroplast transformation.

Examples of non-vascular photosynthetic organisms include bryophtyes, such as marchantiophytes or anthocerotophytes. In some instances the organism is a cyanobacteria. In some instances, the organism is algae (e.g., macroalgae or microalgae). The algae can be unicellular or multicellular algae. In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

The methods of the present invention are exemplified using the microalgae, C. reinhardtii. The use of microalgae to express a polypeptide or protein complex according to a method of the invention provides the advantage that large populations of the microalgae can be grown, including commercially (Cyanotech Corp.; Kailua-Kona Hi.), thus allowing for production and, if desired, isolation of large amounts of a desired product. However, the ability to express, for example, functional polypeptides, including protein complexes, in the chloroplasts of any plant and/or modify the chloroplasts or any plant allows for production of crops of such plants and, therefore, the ability to conveniently produce large amounts of the polypeptides. Accordingly, the methods of the invention can be practiced using any plant having chloroplasts, including, for example, macroalgae, for example, marine algae and seaweeds, as well as plants that grow in soil, for example, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea ultilane), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugar cane (Saccharum spp.), oats, duckweed (Lemna), barley, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals such as azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum are also included. Additional ornamentals useful for practicing a method of the invention include impatiens, Begonia, Pelargonium, Viola, Cyclamen, Verbena, Vinca, Tagetes, Primula, Saint Paulia, Agertum, Amaranthus, Antihirrhinum, Aquilegia, Cineraria, Clover, Cosmo, Cowpea, Dahlia, Datura, Delphinium, Gerbera, Gladiolus, Gloxinia, Hippeastrum, Mesembryanthemum, Salpiglossos, and Zinnia. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga ultilane); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Leguminous plants useful for practicing a method of the invention include beans and peas. Beans include guar, locust bean, fenugreek, soybean, garden beans, cowpea, mung bean, lima bean, fava bean, lentils, chickpea, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Preferred forage and turf grass for use in the methods of the invention include alfalfa, orchard grass, tall fescue, perennial ryegrass, creeping bent grass, and redtop. Other plants useful in the invention include Acacia, aneth, artichoke, arugula, blackberry, canola, cilantro, clementines, escarole, eucalyptus, fennel, grapefruit, honey dew, jicama, kiwifruit, lemon, lime, mushroom, nut, okra, orange, parsley, persimmon, plantain, pomegranate, poplar, radiata pine, radicchio, Southern pine, sweetgum, tangerine, triticale, vine, yams, apple, pear, quince, cherry, apricot, melon, hemp, buckwheat, grape, raspberry, chenopodium, blueberry, nectarine, peach, plum, strawberry, watermelon, eggplant, pepper, cauliflower, Brassica, e.g., broccoli, cabbage, ultilan sprouts, onion, carrot, leek, beet, broad bean, celery, radish, pumpkin, endive, gourd, garlic, snapbean, spinach, squash, turnip, ultilane, chicory, groundnut and zucchini. Thus, the compositions contemplated herein include host organisms comprising any of the above nucleic acids. The host organism can be any chloroplast-containing organism.

The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, seeds, fruits, cuttings, seedlings, tubers, rootstocks, and the like.

Eukaryotic host cells may be a fungal cell (e.g., S. cerevisiae, Sz. pombe or U. maydis). Examples of prokaryotic host cells include E. coli and B. subtilis, cyanobacteria and photosynthetic bacteria (e.g. species of the genus Synechocystis or the genus Synechococcus or the genus Athrospira). Examples of non-vascular plants which may be a host organism (or the source of target DNA) include bryophtyes, such as marchantiophytes or anthocerotophytes. In some instances, the organism is algae (e.g., macroalgae or microalgae, such as Chlamydomonas reinhardtii, Chorella vulgaris, Dunaliella salina, Haematococcus pluvalis, Scenedesmus ssp.). The algae can be unicellular or multicellular algae. In some instances the organism is a rhodophyte, chlorophyte, heterokontophyte, tribophyte, glaucophyte, chlorarachniophyte, euglenoid, haptophyte, cryptomonad, dinoflagellum, or phytoplankton. In other instances One of skill in the art will recognize that these organisms are given merely as examples and other organisms may be substituted where appropriate positive and negative selectable markers are available.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLES

### Example 1

### DNA Purification and Analysis

DNA is isolated and analyzed according to methods known in the art.

To prepare DNA from Chlamydomonas reinhardtii to use as a template for PCR, 10⁶ algae cells (from agar plate or liquid culture) are suspended in 10 mM EDTA and heated to 95° C. for 10 minutes, then cooled to near 23° C. The solution is added to the PCR mixture directly.

To prepare purified chloroplast DNA from Chlamydomonas reinhardtii, 5×10⁸ algae cells are collected from liquid culture by centrifugation at 3000×g for 10 min, washed once with water, centrifuged at 3000×g for 10 min, resuspended in 10 mL of lysis solution (10 mM Tris pH=8.0, 10 mM EDTA, 150 mM NaCl, 2% SDS, 2% Sarkosyl, and 25 ug/mL Pronase (Roche)), and incubated at 37° C. for 1 hour. The lysate is then gently extracted with phenol/chloroform followed by two chloroform washes. Total DNA is isolated by ethanol precipitation and resuspension in resuspension buffer (10 mM Tris pH=7.4, 1 mM EDTA, and 0.1 mg/mL RNase). Chloroplast DNA can be purified by adding of denaturing solution (200 mM NaOH and 1% SDS (w/v)) is added to the resuspended DNA and inverted several times. Neutralizing solution (3.0 M potassium acetate, pH=5.5) is added, mixed, incubated on ice for 10 min and centrifuged at 15000 RPM for 30 min. The supernatant is decanted and applied to a QIAGEN-tip 500 and the DNA is isolated according to the QIAGEN Plasmid Maxi Kit.

An alternative method for preparing purified chloroplast DNA from Chlamydomonas reinhardtii involves embedding algae cells or purified chloroplasts in low-melt agarose plugs to prevent shearing of the DNA. Chloroplasts are isolated by lysing whole cells in a nitrogen decompression chamber and separating intact cells and debris from the chloroplasts by percoll density gradient centrifugation. To lyse the cells and/or chloroplasts, the plugs are incubated at 55° C. for 36 hours in lysis buffer (0.5 M EDTA, 1% Sarkosyl, 0.2 mg/mL proteinase K). When lysis is complete, plugs are washed 3 times with TE, and then stored in storage buffer (10 mM Tris pH=7.4, 1 mM EDTA). To release chloroplast DNA into solution, the plugs are washed 3 times with 30 mM NaCl, and melted at 65° C. for 10 min. The melted plugs are shifted to 42° C. and treated with β-agarase (New England Biolabs) for 1 hour. The solution of DNA can be used directly for downstream applications or ethanol precipitated to concentrate the sample.

To prepare DNA from yeast to use as a template for PCR, 10⁶ yeast cells (from agar plate or liquid culture) are suspended in lysis buffer (6 mM KHPO4, pH=7.5, 6 mM NaCl, 3% glycerol, 1 U/mL zymolyase) and heated to 37° C. for 30 min, 95° C. for 10 minutes, then cooled to near 23° C. The solution is added to the PCR mixture directly.

To prepare plasmid DNA from yeast, desired clones are grown in selective liquid media (e.g., CSM-Trp) to saturation at 30° C. Cells are collected by centrifugation at 3000×g for 10 minutes and resuspended in 150 uL of lysis buffer (1 M sorbitol, 0.1 M sodium citrate, 0.06 M EDTA pH=7.0, 100 mM beta-mercaptoethanol, and 2.5 mg/mL zymolyase). The solution is incubated for 1 hr at 37° C. 300 uL of denaturing solution (1% SDS and 0.2N NaOH) is added and solution is incubated at 60° C. for 15 min. 150 uL of neutralizing solution (3M potassium acetate, pH=4.8) is added and the solution is incubated on ice for 10 min. The solution is centrifuged at 14,000 RPM for 10 min and the supernatant is transferred to another tube. 1 mL of isopropanol is added, the mixture is gently mixed and centrifuged at 14,000 RPM for 10 min. The pellet is washed once with 1 mL of 70% ethanol and centrifuged at 14,000 RPM for 10 min. The DNA pellet is air-dried and resuspended in 60 uL of resuspension buffer (10 mM Tris pH=7.4, 1 mM EDTA, and 0.1 mg/mL RNase).

To prepare plasmid DNA from bacteria, cells are grown to saturation at 37° C. in LB containing the appropriate antibiotic (Kan or Amp). If the DNA of interest contains standard replication elements, cells are harvested by centrifugation. If the DNA of interest contains P1 replication elements, saturated cell cultures are diluted 1:20 in LB+Kan+IPTG and grown for 4 hours at 37° C., then harvested. The Plasmid Maxi kit (QIAGEN) is used to prepare plasmid DNA from the cell pellets.

For illustrative purposes, and without limiting the invention to the specific methods described, DNA samples prepared from algae, yeast, or bacteria (in plugs or in solution) are analyzed by pulse-field gel electrophoresis (PFGE), or digested with the appropriate restriction endonuclease (e.g., SmaI) and analyzed by PFGE, conventional agarose gel electrophoresis, and/or Southern blot. Standard protocols useful for these purposes are fully described in Gemmill et al. (in “Advances in Genome Biology”, Vol. 1, “Unfolding The Genome,” pp 217 251, edited by Ram S. Verma).

One of skill will appreciate that many other methods known in the art may be substituted in lieu of the ones specifically described or referenced.

### Example 2

### Transformation Methods

E. coli strains DH10B or Genehog are made electrocompetent by growing the cells to an OD 600 of 0.7, then collected and washed twice with ice-cold 10% glycerol, flash frozen in a dry-ice ethanol bath and kept at −80° C. Total yeast or algae DNA is prepared and electroporated into E. coli by using, for example, a 0.1 cm cuvette at 1,800 V, 200 ohms and 25 mF in a Bio-Rad Gene Pulsar Electroporator. Cells are allowed to recover and clones are selected on agar growth media containing one or more antibiotics, such as kanamycin (50 μg/mL), ampicillin (100 μg/mL), gentamycin (50 μg/mL), tetracycline (51 μg/mL), or chloramphenicol (34 μg/mL).

Yeast strains YPH857, YPH858 or AB1380 may be transformed by the lithium acetate method as described in Sheistl & Geitz (Curr. Genet. 16:339 346, 1989) and Sherman et al., “Laboratory Course Manual Methods in Yeast Genetics” (Cold Spring Harbor Laboratory Press, Cold Spring Harbor, 1986) or a spheroplast method such as the one described by Sipiczki et al., Curr. Microbiol., 12(3): 169-173 (1985). Yeast transformants are selected and screened on agar media lacking amino and/or nucleic acids, such as tryptophan, leucine, or uracil. Standard methods for yeast growth and phenotype testing are employed as described by Sherman et al., supra.

Algae strains csc137c (mt+), W1.1, or W1-1 may be transformed by particle bombardment. Cells are grown to late log phase (approximately 7 days) in TAP medium in the presence or absence of 0.5 mM 5-fluorodeoxyuridine (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells resuspended in 4 ml TAP medium for subsequent chloroplast transformation by particle bombardment (Cohen et al., supra, 1998).

One of skill will appreciate that many other transformation methods known in the art may be substituted in lieu of the ones specifically described or referenced herein.

### Example 3

### A Hybrid Gap-Filling Vector to Capture a Chloroplast Genome

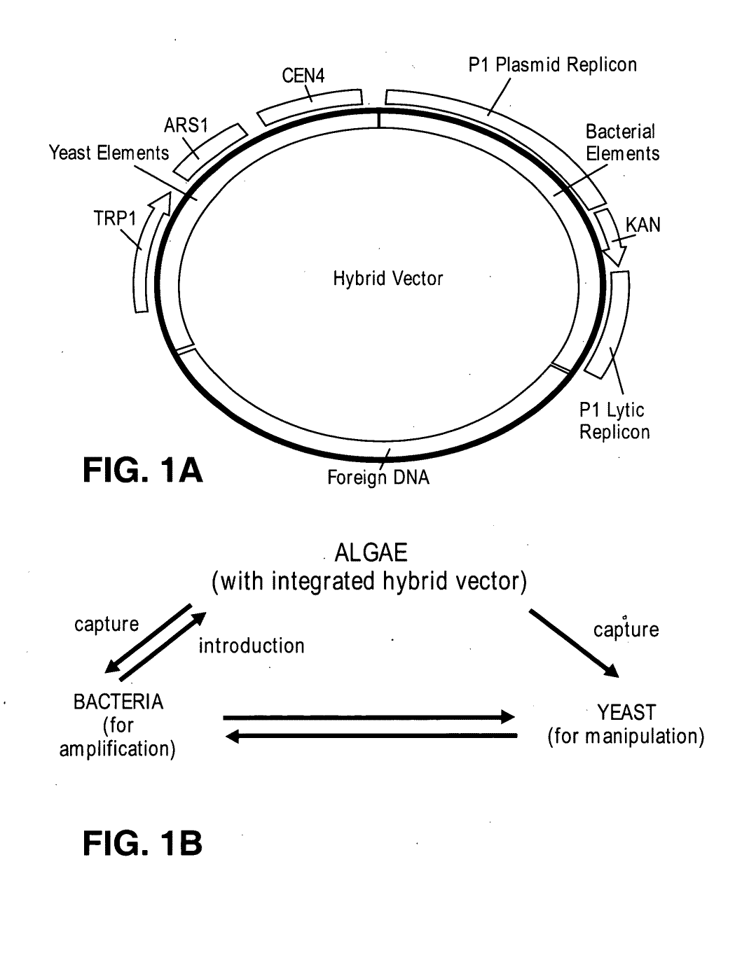

In this example, a system is established using a hybrid gap-filling vector to capture chloroplast DNA (FIG. 1). The hybrid gap filling vector backbone contains yeast elements that allow it to function as a yeast artificial plasmid (YAP) and bacterial elements that allow it to function as a plasmid artificial chromosome (PAC). The yeast elements include a yeast selection marker sequence (e.g. TRP1 or LEU2), a yeast centromere sequence (CEN), and a yeast autonomously replicating nucleotide sequence (ARS). Bacterial elements include a P1 or bacterial origin of replication sequence and a bacterial selection maker sequence (e.g. Kan′).

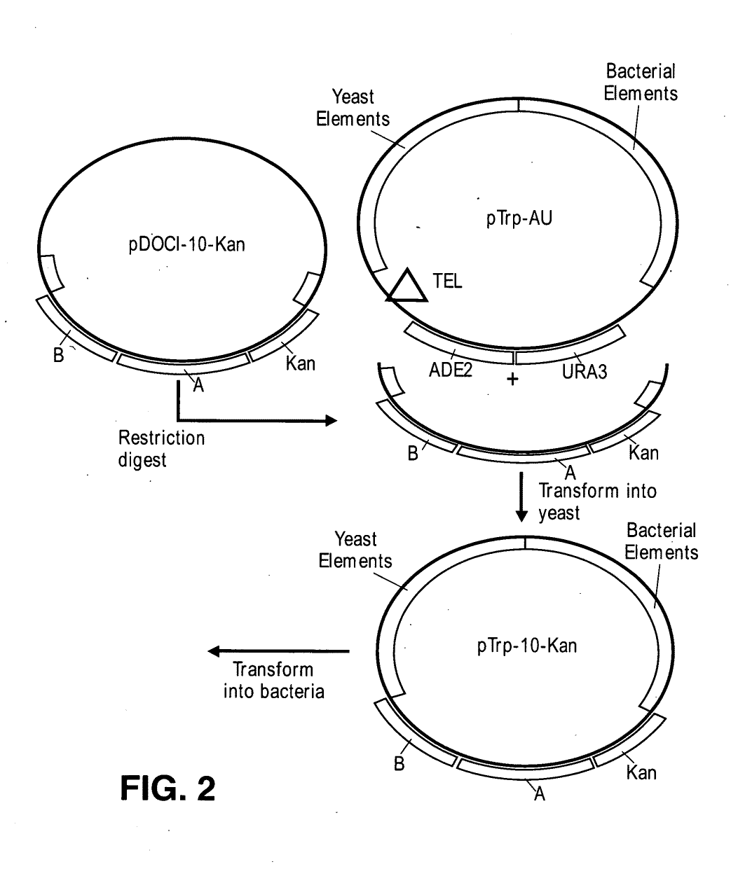

To manipulate the hybrid gap-filling vector, the vector pDOCI (SEQ ID NO. 1) was generated. Portions of pTRP-AU (FIG. 2) were amplified using PCR primer pairs that anneal to sites surrounding the region encompassing TEL, ADE2, and URA3. One pair amplifies a region within the yeast elements (SEQ ID NOs. 21 and 22) and the other pair amplifies a region within the bacterial elements (SEQ ID NOs. 23 and 24). The PCR products were assembled into a single DNA fragment by PCR assembly using a single primer pair (SEQ ID NOs. 21 and 24). The assembled product was digested with NotI and ligated to NotI-digested pUC-SE (SEQ ID NO. 2) to form pDOCI (SEQ ID NO. 1).

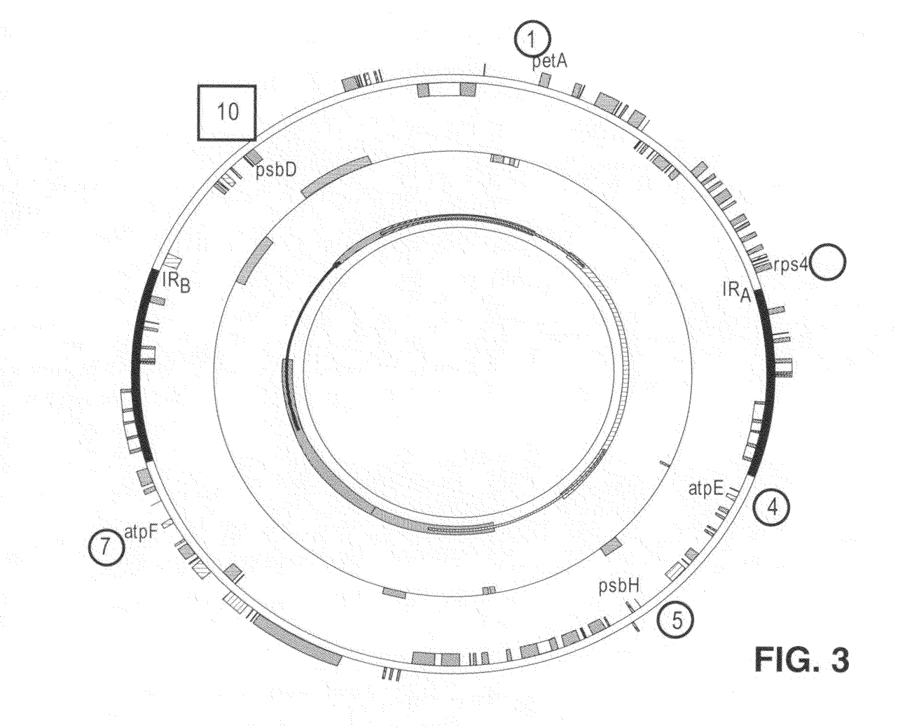

To adapt the hybrid gap-filling vector to capture chloroplast DNA, the vector pDOCI-10 (SEQ ID NO. 3) was generated. Portions of the C. reinhardtii chloroplast genome were PCR amplified using two primer pairs specific for two adjacent regions (SEQ ID NOs. 25 and 26 and SEQ ID NOs. 27 and 28) near the psbD locus. (FIG. 3; indicated by number 10 surrounded by a box). Each PCR product was digested with NotI and I-SceI and ligated to pDOCI (SEQ ID NO. 1) that was digested with I-SceI to form pDOCI-10 (SEQ ID NO. 3).

To adapt pDOCI-10 to confer antibiotic resistance in algae, a selection marker was cloned. pSE-3HB-Kan (SEQ ID NO. 4), which contains a kanamycin resistance encoding gene from bacteria, which is regulated by the 5′ UTR and promoter sequence for the atpA gene from C. reinhardtii and the 3′ UTR sequence for the rbcL gene from C. reinhardtii, was digested with SnaBI, which liberated the kanamycin resistance cassette. The cassette was ligated to SnaBI-digested pDOCI-10 to form pDOCI-10-Kan (SEQ ID NO. 5).

The hybrid gap-filling for capturing chloroplast DNA, pTRP-10-Kan (SEQ ID NO. 6), was constructed using recombination in yeast (FIG. 2). Briefly, pDOCI-10-Kan was digested with PacI and AscI to liberate the cassette that introduces chloroplast genome-specific elements into the hybrid gap-filling vector. This cassette was transformed along with pTRP-AU into the yeast strain YPH858 using the lithium acetate method. Homologous recombination takes place in vivo in the transformed yeast cells. Transformants that correctly integrated with cassette were isolated based on growth on CSM-Trp agar media containing 5-fluorooratic acid (5-FOA) and by red color. 5-FOA selects for clones that lack a functional URA3 gene and the red color results when the ADE2 gene is eliminated. Plasmid DNA was isolated from yeast clones that were grown in CSM-Trp liquid media and transformed into E. coli (DH10B). To generate large amounts of pTRP-10-Kan, DH10B cells harboring pTRP-10-Kan were grown to saturation at 37° C. in LB+Kan (50 ug/mL), and then diluted 1:20 in LB+Kan+IPTG and grown for 4 hours at 37° C. DNA was prepared from the bacterial culture using the Plasmid Maxi kit (QIAGEN).

The composition of the vector was verified by DNA sequencing of the entire plasmid.

### Example 4

### Vectors to Stabilize and/or Modify Chloroplast Genome DNA in an Exogenous Host

Often, large pieces of heterologous DNA are unstable in host organisms such as yeast or bacteria. This may be due to multiple factors, including, but not limited to, the presence of toxic gene products or codon bias and/or lack of selective pressure. Therefore, the target DNA within the shuttle vector may be altered within yeast or bacteria. For example, certain portions of a target DNA sequence (e.g., coding regions or promoters) may be deleted or moved by recombination within the host organism. In a similar way, when a shuttle vector carrying the target DNA is transferred back to the organism (or a closely related species) that donated the target DNA, the target DNA can become unstable.

Such sites of instability and susceptible sequences are readily discovered by determining which pieces of genomic DNA elude capture or disappear over time. These sites can be detected by comparing initially isolated gap-filling target DNA plasmids with plasmids isolated from transformed strains which have been sequentially passaged in the laboratory under conditions which select for the presence of the plasmid-encoded selectable marker (e.g., TRP1) or by comparison with native target DNA (e.g., C. reinhardtii chloroplast DNA). Such comparison may be performed, for example, by restriction fragment length polymorphism (RFLP) analysis or direct sequencing. One of skill in the art will recognize that there are multiple other protocols and methods to determine such differences.

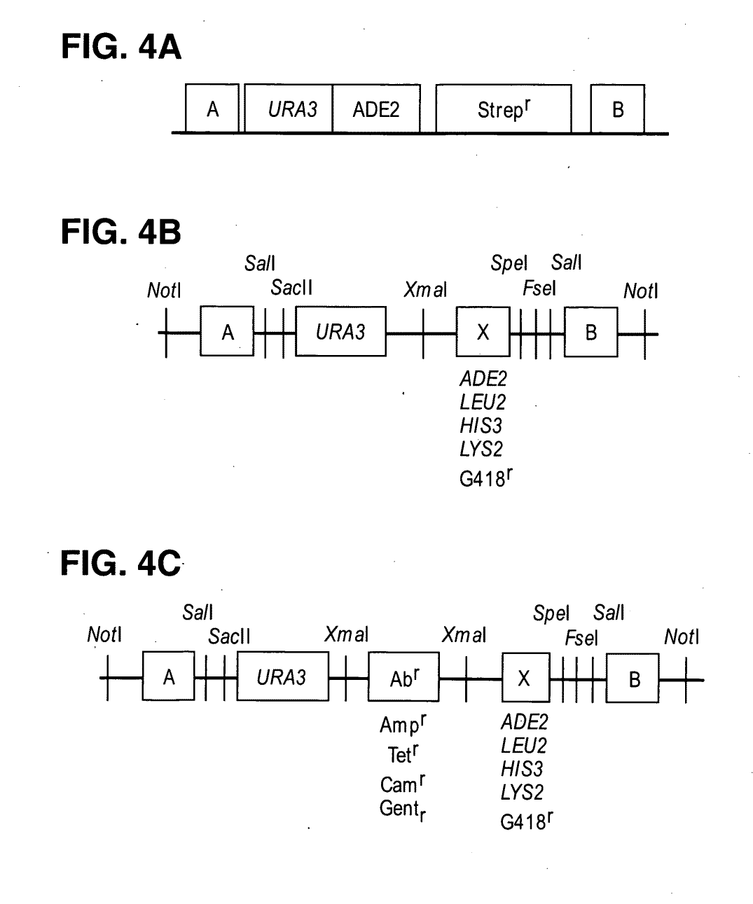

Once identified, such sites and sequences can be forced to remain through selection of markers. FIG. 4 shows examples of arrangements of selectable markers in the multiple cloning site of a vector.

To generate a stabilization vector containing yeast and algae stability elements, the region of DNA in pTrp-AU encompassing the ADE2 and URA3 genes was liberated by digestion with SfiI followed by gel purification of the desired fragment. The fragment was treated with Klenow fragment to create blunt ends and ligated to PmlI-degested pSE-3HB-Strep (SEQ ID NO. 7), creating pSE-3HB-Strep-AU (FIG. 4B). pSE-3HB-Strep is a vector that targets a streptomycin resistance encoding gene from bacteria, which is regulated by the 5′ UTR and promoter sequence for the atpA gene from C. reinhardtii and the 3′ UTR sequence for the rbcL gene from C. reinhardtii, to the 3HB locus of the C. reinhardtii chloroplast genome.

To generate vectors that target other regions of the chloroplast genome, 800-1000 bp regions were amplified using PCR primer pairs that anneal to 5′ and 3′ regions flanking the sites indicated by numbered circles in FIG. 3 (site 1-5′, SEQ ID NOs. 29 and 30; site 1-3′, SEQ ID NOs. 31 and 32; site 3-5′, SEQ ID NOs. 33 and 34; site 3-3′, SEQ ID NOs. 35 and 36; site 4-5′, SEQ ID NOs. 37 and 38; site 4-3′, SEQ ID NOs. 39 and 40; site 5-5′, SEQ ID NOs. 41 and 42; site 5-3′, SEQ ID NOs. 43 and 44; and site 7-5′, SEQ ID NOs. 45 and 46; site 7-3′, SEQ ID NOs. 47 and 48). Each pair of PCR products was digested with NotI and I-SceI, mixed, and ligated to NotI-digested pUC-SE (SEQ ID NO. 2), producing plasmids pUC1, pUC3, pUC4, pUC5 and pUC7 (named for their position of integration).

Pairs of yeast selection markers were constructed so that multiple stabilization sites could be employed simultaneously (FIG. 4B). Each marker pair contains the URA3 gene (SEQ ID NO. 8), which was PCR amplified from pRS416. Each marker pair also contains the LEU2 gene (SEQ ID NO. 9) amplified from pRS415, the HIS3 gene (SEQ ID NO. 10) amplified from pRS413, the ADE2 gene (SEQ ID NO. 11) amplified from pTrp-AU, the LYS2 gene (SEQ ID NO. 12) amplified from S. cerevisiae genomic DNA, or the kanMX6 gene (SEQ ID NO. 13) from pFA6a-kanMX6, which confers resistance to the antifungal agent G418. The primers used for the URA3 gene. add the XmaI restriction site to the 5′ end (SEQ ID NO. 49) and SalI and SacII to the 3′ end (SEQ ID NO. 50). The primers used for the LEU2, HIS3, ADE2, LYS2, and G418^{r} genes add the XmaI restriction site to the 5′ end (SEQ ID NO. 51 for LEU2, SEQ ID NO. 52 for HIS3, SEQ ID NO. 53 for ADE2, SEQ ID NO. 54 for LYS2, and SEQ ID NO. 55 for G418^{r}) and SalI, FseI, and SpeI sites to the 3′ end (SEQ ID NO. 56 for LEU2, SEQ ID NO. 57 for HIS3, SEQ ID NO. 58 for ADE2, SEQ ID NO. 59 for LYS2, and SEQ ID NO. 60 for G418^{r}). Each PCR product was digested with XmaI and SalI, mixed pairwise, and ligated into the desired integration vector, resulting in pUC1-URA3/ADE2, pUC3-URA3/LEU2, pUC4-URA3/HIS3, pUC5-URA3/ADE2, and pUC7-URA3/LYS2. URA3 is used in each case because it allows for positive and negative selection. Marker pairs can be introduced based on selection for either gene (in the case of a single modification), the non-URA3 gene in the case of two or more modification. Then the markers can be removed by introducing DNA with terminal sequences homologous to those surrounding the marker pairs and selecting for growth on minimal media containing 5-FOA.

To promote sequence stability in bacteria, antibiotic resistance markers were cloned into the yeast selection marker pairs. The bacterial stability markers include, but are not limited to, the ampicillin resistance gene (Amp^{r}, SEQ ID NO. 14) amplified from pET-21a, the tetracycline resistance gene (Tet^{r}, SEQ ID NO. 15) amplified from pBR322, the chloramphenicol resistance gene (Cam^{r}, SEQ ID NO. 16) amplified from pETcoco-1, and the gentamycin resistance gene (Gent^{r}, SEQ ID NO. 17) amplified from pJQ200. For each gene, primer pairs (SEQ ID NOs. 61 and 62 for Amp^{r}, SEQ ID NOs. 63 and 64 for Tet^{r}, SEQ ID NOs. 65 and 66 for Cam^{r}, and SEQ ID NOs. 67 and 68 for Gent) that add XmaI sites to both the 5′ and 3′ ends were used to PCR amplify the antibiotic resistance fragment. Each PCR product was digested with XmaI and ligated into XmaI-digested pUC1-URA3/ADE2, pUC3-URA3/LEU2, pUC4-URA3/HIS3, pUC5-URA3/ADE2, and pUC7-URA3/LYS2. FIG. 4C shows the arrangement of the yeast and bacterial stability markers.

### Example 5

### Introduction of Hybrid and Stabilization Vectors into a Chloroplast Genome

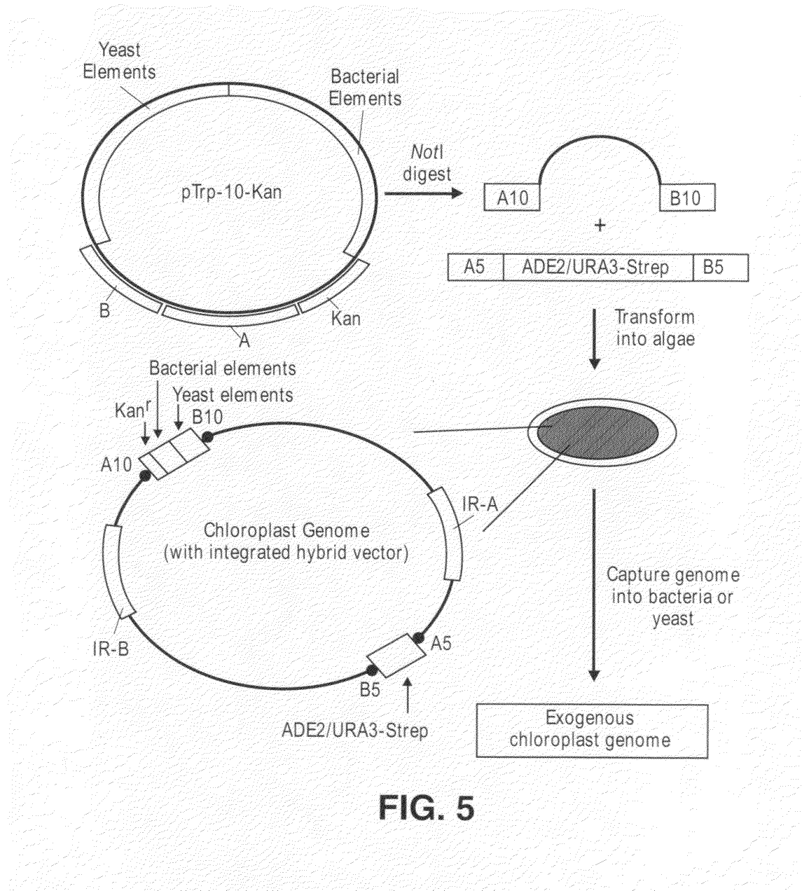

To generate a C. reinhardtii chloroplast genome that contains a hybrid vector with or without a stabilization vector, pTRP-10-Kan and pSE-3HB-Strep-AU were transformed into algae cells. pTRP-10-Kan was digested with NotI to linearize the vector such that the chloroplast targeting elements on are on each end (FIG. 5). pSE-3HB-Strep-AU was transformed as circular DNA.

For these experiments, all transformations are carried out on C. reinhardtii strain 137c (mt+). Cells are grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells resuspended in 4 ml TAP medium for subsequent chloroplast transformation by particle bombardment (Cohen et al., supra, 1998). All transformations are carried out under kanamycin selection (100 μg/ml) in which resistance is conferred by the gene encoded by the segment in FIG. 5 labeled “Kan.” (Chlamydomonas Stock Center, Duke University).

PCR is used to identify transformed strains. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl2, dNTPs, PCR primer pair(s), DNA polymerase, and water is prepared. Algae lysate in EDTA is added to provide a template for the reaction. The magnesium concentration is varied to compensate for amount and concentration of algae lysate and EDTA added. Annealing temperature gradients are employed to determine optimal annealing temperature for specific primer pairs.

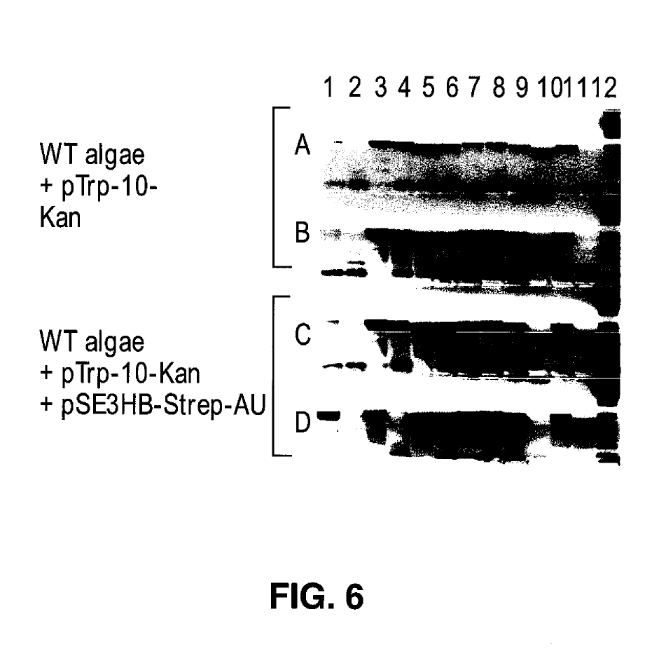

FIG. 6 shows examples of isolated algae strains that contain pTrp-10-Kan with and without pSE-3HB-Strep-AU. Strains that integrated pTRP-10-Kan were identified using any of a set of primer pairs that amplify different regions in the vector backbone (Lane 1, SEQ ID NOs. 69 and 70; Lane 2, SEQ ID NOs. 71 and 72; Lane 3, SEQ ID NOs. 73 and 74; Lane 4, SEQ ID NOs. 75 and 76; Lane 5, SEQ ID NOs. 77 and 78; Lane 6, SEQ ID NOs. 79 and 80; Lane 7, SEQ ID NOs. 81 and 82; Lane 8, SEQ ID NOs. 83 and 84; and Lane 9, SEQ ID NOs. 85 and 86) or primer pairs that span the junction between pTrp-10-Kan DNA and chloroplast genome DNA (Lane 10, SEQ ID NOs. 87 and 88; Lane 11, SEQ ID NOs. 89 and 90). To identify strains that have integrated pSE-3HB-Strep-AU, primer pairs (SEQ ID NOs. 91 and 92) were used which amplify regions within the ADE2 gene. Desired clones are those that yield PCR products of expected size.

### Example 6

### Capture of a Chloroplast Genome into an Exogenous Host

In this example, C. reinhardtii chloroplast DNA is isolated using standard techniques as described above. C. reinhardtii chloroplast DNA containing the pTRP-10-Kan vector with or without pSE-3HB-Strep-AU was used to transform bacteria. Electrocompetent E. coli strains DH 10B or Genehog were transformed and selected on LB agar growth medium with kanamycin (50 mg/l). DNA from individual clones was isolated by growing the cells to saturation at 37° C. in LB liquid growth medium with kanamycin (50 mg/l). Saturated cell cultures are diluted 1:20 in LB+Kan+IPTG and grown at 37° C. for 4 hours. The Plasmid Maxi kit (QIAGEN) is used to prepare plasmid DNA from the isolated clones.

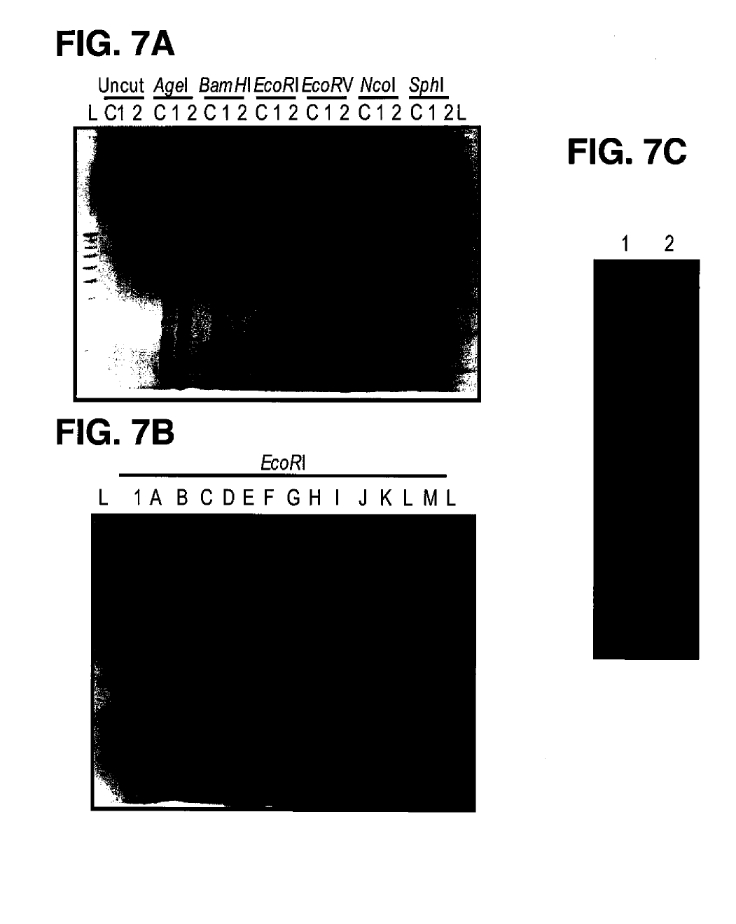

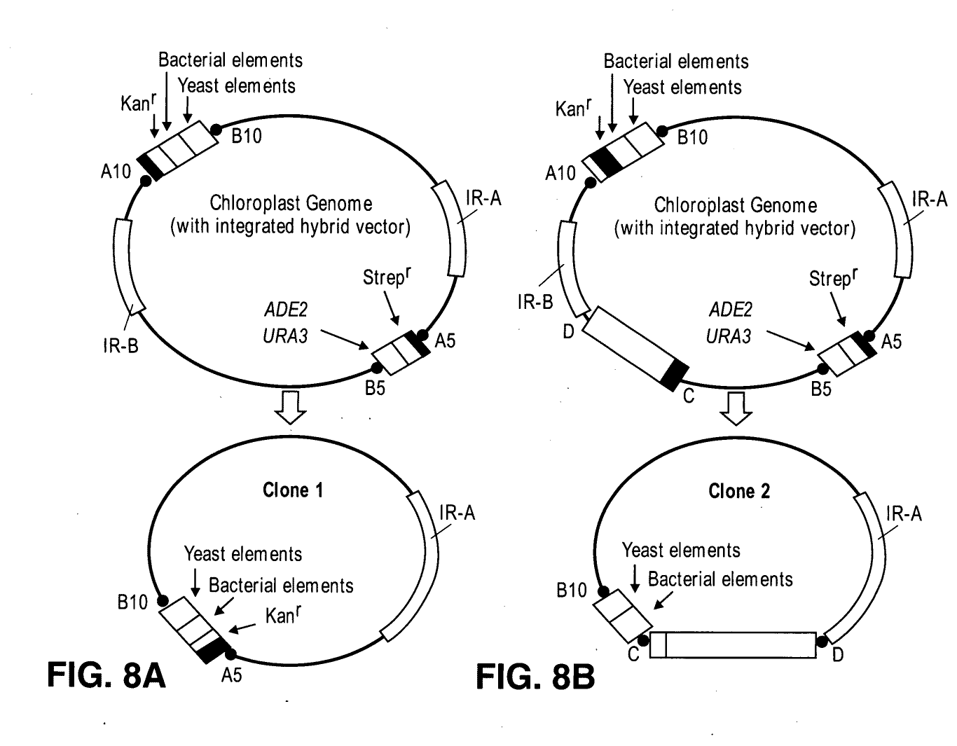

FIG. 7A shows the restriction analysis of two types of clones obtained from bacterial transformation (Clone 1 and Clone 2) compared to the parent hybrid vector (Clone C). Clones 1 and 2 are composed of >100 kb of DNA while the parent hybrid vector is composed of only 23 kb of DNA, demonstrating that large portions of DNA were indeed captured by the hybrid vector. Restriction mapping indicates that Clone I comprises approximately half of the chloroplast genome (FIG. 8A). DNA sequencing of the regions flanking the hybrid vector indicate that a recombination event occurred between the 3′ UTR of the C. reinhardtii kanamycin resistance cassette (which is the 3′ UTR from the rbcL gene in C. reinhardtii) and the 3′ UTR of the C. reinhardtii streptomycin resistance cassette (which is the 3′ UTR from the rbcL gene in C. reinhardtii). Clone 1 retained the C. reinhardtii kanamycin resistance cassette, but lost the C. reinhardtii streptomycin resistance cassette. Restriction mapping indicates that Clone 2 also comprises approximately half of the chloroplast genome (FIG. 8B). However, DNA sequencing of the regions flanking the hybrid vector indicate that a different recombination event occurred to give rise to Clone 2. The 5′ UTR of the C. reinhardtii kanamycin resistance cassette (which is the 5′ UTR from the atpA gene in C. reinhardtii) recombined with the 5′ UTR of the atpA gene in the C. reinhardtii chloroplast genome and the inverted repeat B (IR-B in FIG. 8) recombined with inverted repeat A (IR-A in FIG. 8). Clone 2 lost both the C. reinhardtii kanamycin resistance cassette and the C. reinhardtii streptomycin resistance cassette.

To demonstrate that the hybrid vector would support stable replication in yeast, Clone 1 was transformed into the yeast strain AB1380 by the lithium acetate method and transfomants were selected on CSM-Trp agar media. Transformants were PCR screened using a primer pair (SEQ ID NOs 97 and 98) that amplifies a region with the chloroplast genome DNA of Clone 1. Desired clones are those that give rise to a PCR product of expected size. Individual PCR-positive clones were streaked to generate multiple clones per isolate. DNA was prepared from the isolated yeast clones and transformed into bacteria. Bacteria were PCR screened using a primer pair (SEQ ID NOs 97 and 98) that amplifies a region within the chloroplast genome DNA of Clone 1. Desired clones are those that give PCR products of expected size. DNA was prepared from the isolated bacterial clones and analyzed by restriction digest with EcoRI. FIG. 7B shows that all isolated clones have restriction maps that are identical to the originally isolated Clone 1. Thus, the hybrid vector supports stable replication in yeast.

To determine if the captured DNA is indeed chloroplast genome DNA, a Southern blot was performed. Clones 1 and 2 prepared from bacteria were digested with EcoRI, separated by gel electrophoresis, transferred to a membrane, and probed with radioactive HindIII-digested total DNA from C. reinhardtii. FIG. 7C shows that the DNA in Clones 1 and 2 give rise to a signal, thus indicating that the captured DNA is chloroplast genome DNA.

### Example 7

### Reintroduction of Chloroplast Genome DNA into Algae

For these experiments, all transformations are carried out on either C. reinhardtii strain W1.1, which expresses SAA from the endogenous psbA loci, or W1-1, which expresses LuxAB from the endogenous psbA loci. Both W1.1 and W1-1 are resistant to spectinomycin by virtue of transformation of p228, which introduces a mutation in the 16S rRNA. Cells are grown to late log phase (approximately 7 days) in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells resuspended in 4 ml HSM medium for subsequent chloroplast transformation by particle bombardment (Cohen et al., supra, 1998). All transformations are carried out on HSM agar.

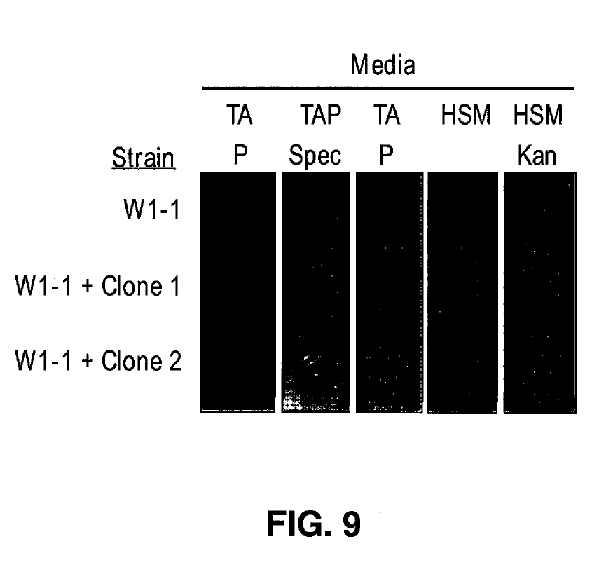

Transformants were selected by growth on HSM, indicating that function of the psbA gene locus was restored. Clones were also subsequently checked for growth on TAP, TAP with spectinomycin (150 μg/ml), TAP with kanamycin (100 μg/ml), HSM, and HSM with kanamycin (100 μg/ml). FIG. 9 shows that W1-1 transformed with Clone 1 is able to grow on all media types. FIG. 9 also shows that W1-1 transformed with Clone 2 is unable to grow on media containing kanamycin, which is expected since Clone 2 does not contain a Kan resistance marker.

### Example 8

### Modification of Chloroplasts for Producing Biomass-Degrading Enzymes

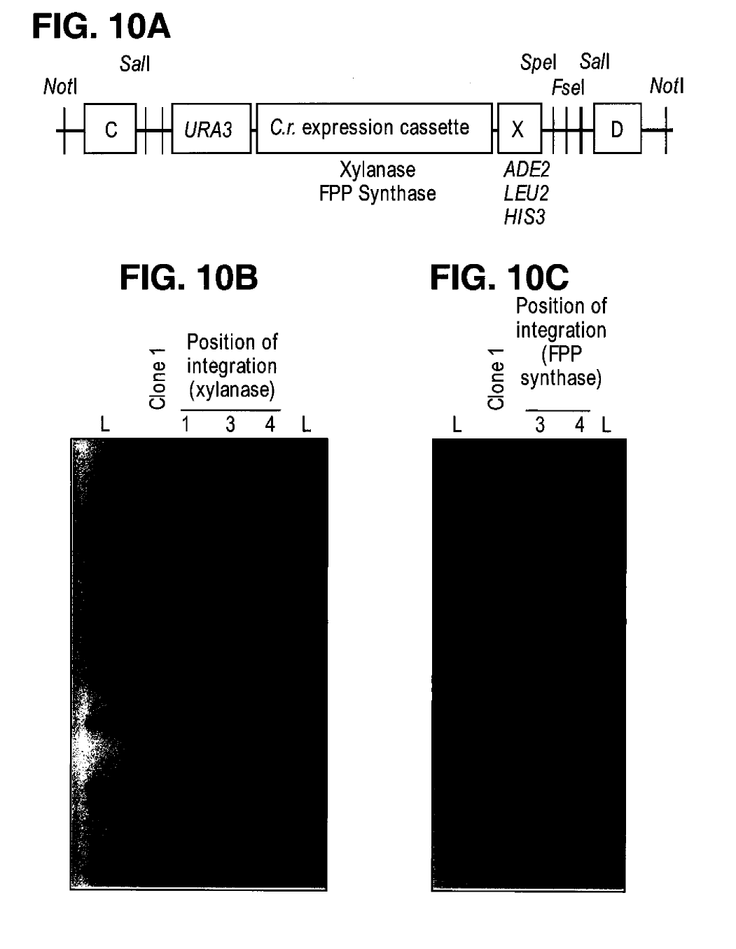

To modify captured chloroplast genome DNA for production of a xylanase, an algae expression cassette was cloned into the yeast marker vectors described in EXAMPLE 4. Briefly, a C. reinhardtii chloroplast expression vector was digested with SpeI to liberate a fragment of DNA (SEQ ID NO. 18) with xylanase from T. reesei regulated by the 5′ UTR for the psbD gene from C. reinhardtii and the 3′ UTR for the psbA gene from C. reinhardtii. The fragment was treated with Klenow fragment to create blunt ends and cloned into SmaI (XmaI) site between the yeast markers in pUC1-URA3/ADE2, pUC3-URA3/LEU2, and pUC4-URA3/HIS3. FIG. 10A shows the arrangement of the various elements in the new vectors.

Chloroplast genome DNA was modified by transforming the modification vector into yeast that harbored the captured DNA. For transformation, all modification vectors were linearized by digestion with NotI. Yeast harboring Clone 1 (from EXAMPLE 6) were grown to saturation in CSM-Trp medium at 30° C., then diluted 1:20 in YPAD medium and grown for 4 hours at 30° C. Yeast were transformed using the lithium-acetate method and transformants were selected for by growth on CSM-Ura medium. Transformants were propagated on CSM-Trp-Ura medium and then PCR screened using primers specific for the xylanase expression cassette (SEQ ID NOs 95 and 96) and a region within the chloroplast genome DNA (SEQ ID NOs 97 and 98). Desired clones are those that give PCR products of expected size for both reactions. DNA was prepared from the isolated yeast clones and transformed into bacteria. Bacteria were PCR screened using primers specific for the xylanase expression cassette (SEQ ID NOs 95 and 96) and a region within the chloroplast genome DNA (SEQ ID NOs 97 and 98). Desired clones are those that give PCR products of expected size for both reactions. DNA was prepared from the isolated bacterial clones and analyzed by restriction digest with EcoRI. FIG. 10B shows that clones were isolated that have restriction maps that are consistent with integration of the xylanase expression cassette in the desired position.

For these experiments, all transformations are carried out on either C. reinhardtii strain W1.1, which expresses SAA from the endogenous psbA loci, or W1-1, which expresses LuxAB from the endogenous psbA loci. Both W1.1 and W1-1 are resistant to spectinomycin by virtue of transformation of p228, which introduces a mutation in the 16S rRNA. Cells are grown to late log phase (approximately 7 days) in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells resuspended in 4 ml HSM medium for subsequent chloroplast transformation by particle bombardment (Cohen et al., supra, 1998). All transformations are carried out on HSM agar.

Transformants were identified by growth on HSM, indicating that function of the psbA gene locus was restored. PCR is used to identify transformants that also contain the endoxylanase expression cassette. For PCR analysis, 10⁶ algae cells (from agar plate or liquid culture) are suspended in 10 mM EDTA and heated to 95° C. for 10 minutes, then cooled to near 23° C. A PCR cocktail consisting of reaction buffer, MgCl2, dNTPs, PCR primer pair(s) (Table 2), DNA polymerase, and water is prepared. Algae lysate in EDTA is added to provide a template for the reaction. The magnesium concentration is varied to compensate for amount and concentration of algae lysate and EDTA added. Annealing temperature gradients are employed to determine optimal annealing temperature for specific primer pairs.

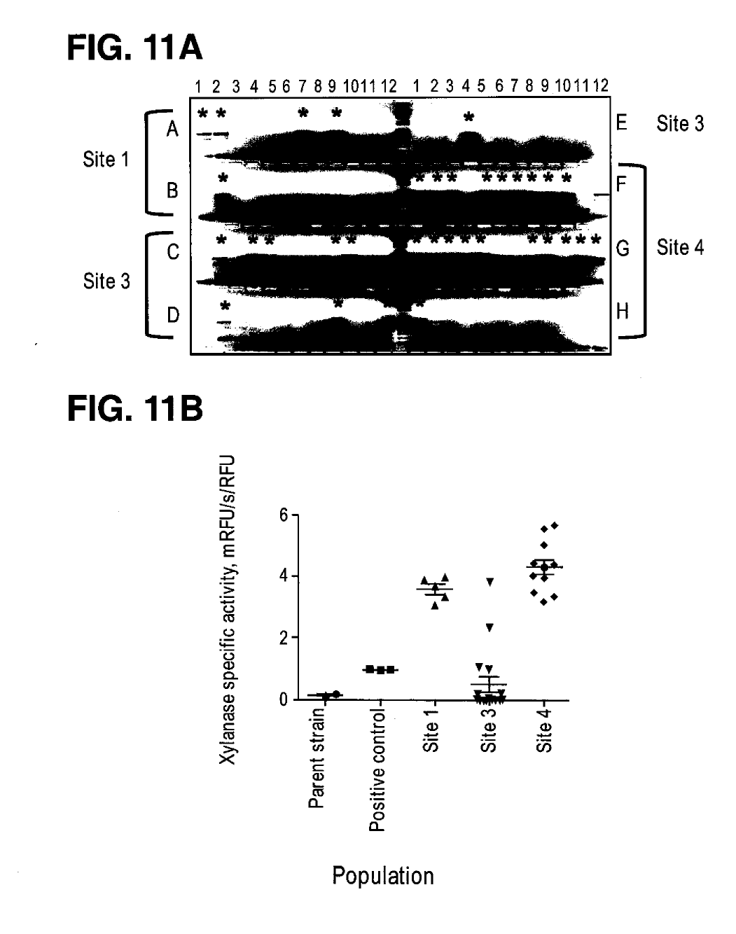

To identify strains that have the endoxylanase expression cassette, a primer pair (SEQ ID NOs 95 and 96) was used which amplifies a region within the endoxylanase expression cassette. Desired clones are those that yield PCR products of expected size. FIG. 11A shows that multiple algae strains were obtained that contain the endoxylanase expression cassette (PCR products indicated with asterisk).

To determine whether functional xylanase is expressed, enzyme activity is examined. Patches of cells (approximately 2 mg per patch) from TAP agar plates containing kanamycin (100 μg/1 mL) were resuspended in 50 ul of 100 mM sodium acetate pH 4.8 in a round bottom 96 well plate (Corning). Resuspended cells were lysed by addition of 20 ul of BugBuster Protein Extraction Reagent (Novagen) and shaken for five minutes at room temperature. Cell lysate (50 ul) was transferred to a black 96 well plate, and the chlorophyll fluorescence of the resulting wells was measured in a SpectraMax M2 microplate reader (Molecular Devices), with an excitation wavelength of 440 nm and an emission wavelength of 740 nm, with a 695 nm cutoff filter. The measured chlorophyll signal in RFUs (relative fluorescence units) was used to normalize the xylanase activity signal to the amount of biomass added to the reaction.

After measurement of the chlorophyll fluorescence, xylanase substrate was added. EnzCheck Ultra Xylanase substrate (Invitrogen) was dissolved at a concentration of 50 ug/ml in 100 mM sodium acetate pH 4.8, and 50 ul of substrate was added to each well of the microplate. The fluorescent signal was measured in a SpectraMax M2 microplate reader (Molecular Devices), with an excitation wavelength of 360 nm and an emission wavelength of 460 nm, without a cutoff filter and with the plate chamber set to 42 degrees Celsius. The fluorescence signal was measured for 15 minutes, and the enzyme velocity was calculated with Softmax Pro v5.2 (Molecular Devices). Enzyme velocities were recorded as RFU/minute. Enzyme specific activities were calculated as milliRFU per minute per RFU of chlorophyll fluorescence. FIG. 11B shows that multiple algae strains containing the xylanase expression cassette at site 1, 3, and 4 (see FIGS. 3 and 10) were obtained that produce functional xylanase enzyme.

### Example 9

### Modification of a Chloroplast Genome to Produce Terpenes

To modify captured chloroplast genome DNA for production of an FPP synthase, an algae expression cassette was cloned into the yeast marker vectors described in EXAMPLE 4. Briefly, a C. reinhardtii chloroplast expression vector was digested with SpeI to liberate a fragment of DNA (SEQ ID NO. 19) with FPP synthase from G. gallus regulated by the 5′ UTR for the psbD gene from C. reinhardtii and the 3′ UTR for the psbA gene from C. reinhardtii. The fragment was treated with Klenow fragment to create blunt ends and cloned into SmaI (XmaI) site between the yeast markers in pUC1-URA3/ADE2, pUC3-URA3/LEU2, and pUC4-URA3/HIS3. FIG. 10A shows the arrangement of the various elements in the new vectors.

Chloroplast genome DNA was modified by transforming the modification vector into yeast that harbored the captured DNA. For transformation, all modification vectors were linearized by digestion with NotI. Yeast harboring Clone 1 (from EXAMPLE 6) were grown to saturation in CSM-Trp media at 30° C., then diluted 1:20 in YPAD and grown for 4 hours at 30° C. Yeast were transformed using the lithium-acetate method and transformants were selected for by growth on CSM-Ura media. Transformants were propagated on CSM-Trp-Ura media and then PCR screened using primers specific for the FPP synthase expression cassette (SEQ ID NOs 95 and 99) and a region within the chloroplast genome DNA (SEQ ID NOs 97 and 98). Desired clones are those that gave PCR products of expected size for both reactions. DNA was prepared from the isolated yeast clones and transformed into bacteria. Bacteria were PCR screened using primers specific for the FPP synthase expression cassette (SEQ ID NOs 95 and 99) and a region within the chloroplast genome DNA (SEQ IDs 97 and 98). Desired clones are those that gave PCR products of expected size for both reactions. DNA was prepared from the isolated bacterial clones and analyzed by restriction digest. FIG. 10C shows that clones were isolated that have restriction maps that are consistent with integration of the FPP synthase expression cassette in the desired position.

For these experiments, all transformations are carried out on either C. reinhardtii strain W1.1, which expresses SAA from the endogenous psbA loci, or W1-1, which expresses LuxAB from the endogenous psbA loci. Both W1.1 and W1-1 are resistant to spectinomycin by virtue of transformation of p228, which introduces a mutation in the 16S rRNA. Cells are grown to late log phase (approximately 7 days) in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells are harvested by centrifugation at 4,000×g at 23° C. for 5 min. The supernatant is decanted and cells resuspended in 4 ml HSM medium for subsequent chloroplast transformation by particle bombardment (Cohen et al., supra, 1998). All transformations are carried out on HSM agar.

Transformants were identified by growth on HSM, indicating that function of the psbA gene locus was restored. PCR is used to identify transformants that also contain the FPP synthase expression cassette. For PCR analysis, 106 algae cells (from agar plate or liquid culture) are suspended in 10 mM EDTA and heated to 95° C. for 10 minutes, then cooled to near 23° C. A PCR cocktail consisting of reaction buffer, MgCl2, dNTPs, PCR primer pair(s), DNA polymerase, and water is prepared. Algae lysate in EDTA is added to provide a template for the reaction. The magnesium concentration is varied to compensate for amount and concentration of algae lysate and EDTA added. Annealing temperature gradients are employed to determine optimal annealing temperature for specific primer pairs.

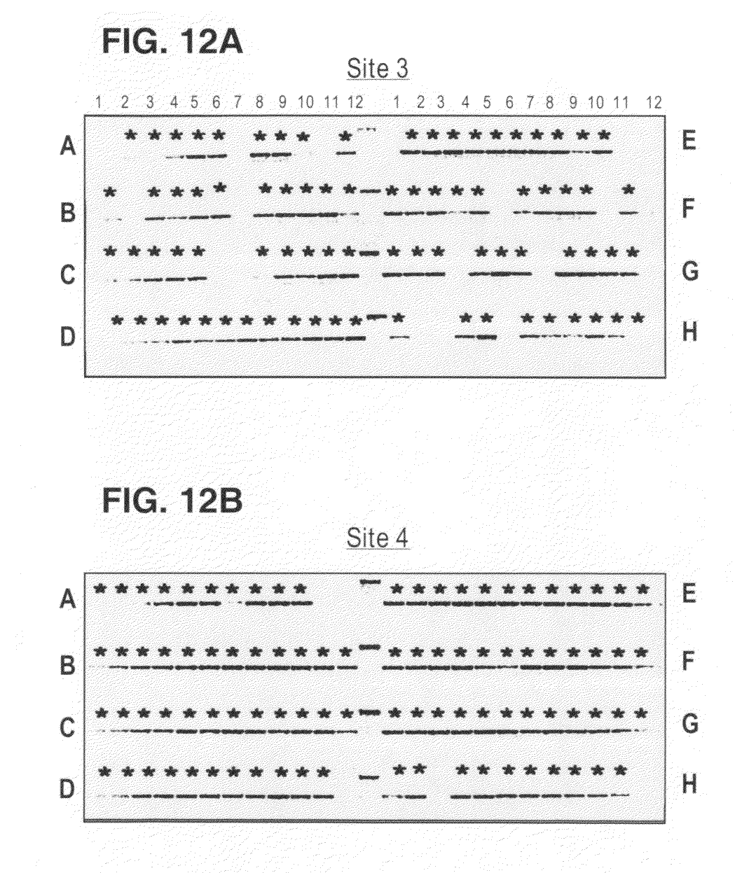

To identify strains that have the FPP synthase expression cassette, a primer pair was used which amplifies a region within the endoxylanase expression cassette. Desired clones are those that yield PCR products of expected size. Multiple algae strains were obtained that contain the FPP synthase expression cassette at (FIG. 12, PCR products indicated with asterisk).

### Example 10

### Gap-Filling a Partial Chloroplast Genome

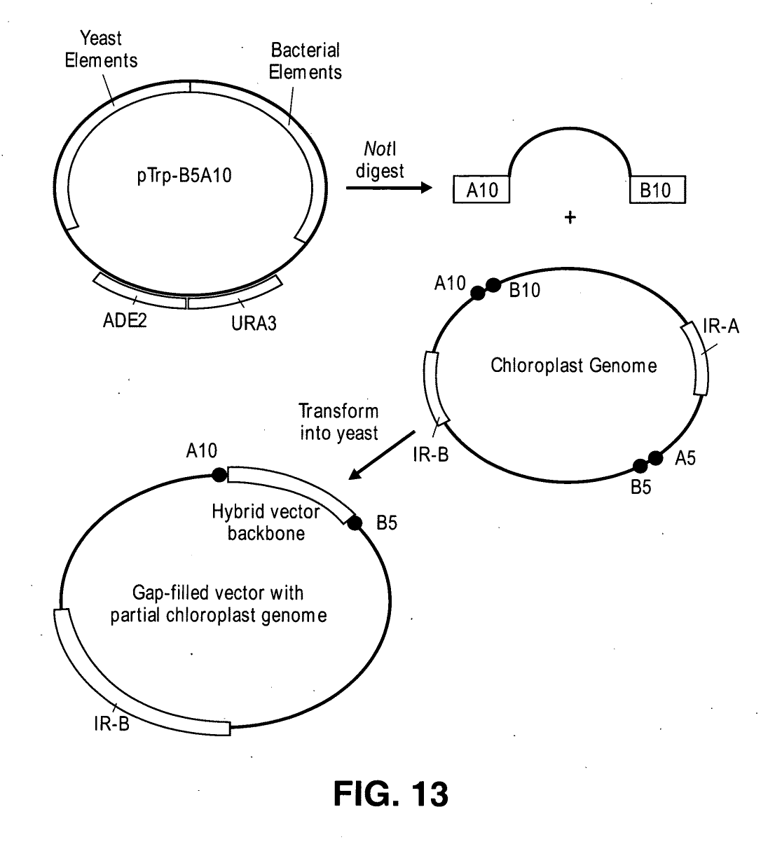

In this example, a system is established using a hybrid gap-filling vector to capture chloroplast DNA by recombination in yeast (FIG. 13). To adapt the hybrid gap-filling vector to capture chloroplast DNA, the vector pDOCI-B5A10 (SEQ ID NO. 20) was generated. Portions of the C. reinhardtii chloroplast genome were PCR amplified using two primer pairs specific for a region adjacent the psbD gene (A10, SEQ ID NOs. 27 and 28) and a region adjacent the near the psbH gene (B5, SEQ ID NOs. 43 and 44). Each PCR product was digested with NotI and I-SceI and ligated to pDOCI (SEQ ID NO. 1) that was digested with I-SceI to form pDOCI-B5A10 (SEQ ID NO. 20).

The hybrid gap-filling for capturing chloroplast DNA, pTRP-B5A10, was constructed using recombination in yeast. Briefly, pDOCI-B5A10 (SEQ ID NO. 20) was digested with PacI and AscI to liberate the cassette that introduces chloroplast genome-specific elements into the hybrid gap-filling vector. This cassette was transformed along with pTRP-AU into the yeast strain YPH858 using the lithium acetate method. Homologous recombination takes place in vivo in the transformed yeast cells. Transformants that correctly integrated with cassette were isolated based on growth on CSM-Trp agar media containing 5-fluorooratic acid (5-FOA) and by red color. 5-FOA selects for clones that lack a functional URA3 gene and the red color results when the ADE2 gene is eliminated. Plasmid DNA was isolated from yeast clones that were grown in CSM-Trp liquid media and transformed into E. coli (DH10B). To generate large amounts of pTRP-10-Kan, DH10B cells harboring pTRP-10-Kan were grown to saturation at 37° C. in LB+Kan (50 ug/mL), and then diluted 1:20 in LB+Kan+IPTG and grown for 4 hours at 37° C. DNA was prepared from the bacterial culture using the Plasmid Maxi kit (QIAGEN).

The desired genomic DNA is captured by using the sites in a gap filling vector that have high homology to the regions of the target genomic DNA (indicated by A10 and B5 in FIG. 13). Linearized gap filling vector DNA and chloroplast genome DNA are used to transform S. cerevisiae using the lithium acetate or spheroplast methods as described in EXAMPLE 2. Homologous recombination takes place in vivo in the transformed yeast cells. Once the target DNA is captured by the vector via homologous recombination, the DNA can be stably replicated in both yeast and bacterial systems. As indicated in FIG. 1, the gap filling vector contains the selectable marker TRP1. Therefore, the yeast cells are plated on tryptophan-free medium to screen for positive transformants. Transformed strains growing on the tryptophan-free medium are screened for the presence of target DNA inserts in the gap filling plasmid. Such screening can be by any known method in the art, for example, restriction enzyme digestion, PCR and/or gel electrophoresis.

Yeast transformants screened by PCR using primers specific for chloroplast genome DNA (SEQ IDs 89 and 90). Desired clones are those that give a PCR product of expected size. DNA is prepared from the isolated yeast clones and transformed into bacteria. Bacterial transformants are PCR screened using primers specific for chloroplast genome DNA (SEQ IDs 89 and 90). Desired clones are those that give a PCR product of expected size. DNA is prepared from the isolated bacterial clones and analyzed by restriction digest with EcoRI.

### Example 11

### Gap-Filling a Complete Chloroplast Genome

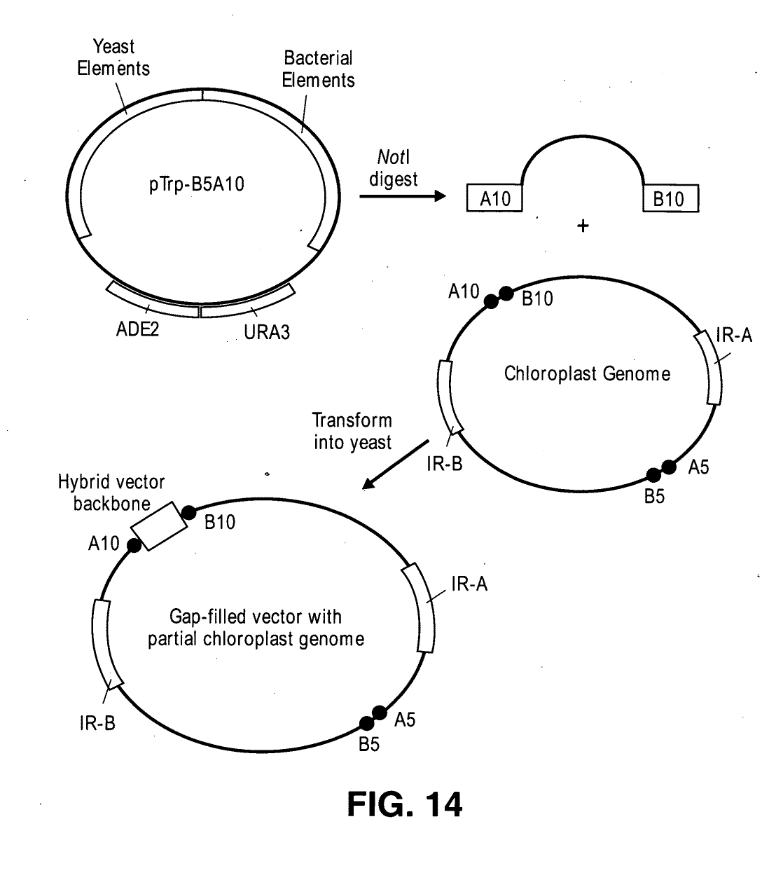

In this example, a system is established using a hybrid gap-filling vector to capture a complete chloroplast genome by recombination in yeast (FIG. 14). The hybrid gap-filling for capturing chloroplast DNA, pTRP-10, was constructed using recombination in yeast. Briefly, pDOCI-10 (described in EXAMPLE 3, SEQ ID NO. 3) was digested with PacI and AscI to liberate the cassette that introduces chloroplast genome-specific elements into the hybrid gap-filling vector. This cassette was transformed along with pTRP-AU into the yeast strain YPH858 using the lithium acetate method. Homologous recombination takes place in vivo in the transformed yeast cells. Transformants that correctly integrated with cassette were isolated based on growth on CSM-Trp agar media containing 5-fluorooratic acid (5-FOA) and by red color. 5-FOA selects for clones that lack a functional URA3 gene and the red color results when the ADE2 gene is eliminated. Plasmid DNA was isolated from yeast clones that were grown in CSM-Trp liquid media and transformed into E. coli (DH10B). To generate large amounts of pTRP-10-Kan, DH10B cells harboring pTRP-10-Kan were grown to saturation at 37° C. in LB+Kan (50 ug/mL), and then diluted 1:20 in LB+Kan+IPTG and grown for 4 hours at 37° C. DNA was prepared from the bacterial culture using the Plasmid Maxi kit (QIAGEN).

The desired genomic DNA is captured by using the sites in a gap filling vector that have high homology to the adjacent regions of the target genomic DNA (indicated by A10 and B 10 in FIG. 14). Linearized gap filling vector DNA and chloroplast genome DNA are used to transform S. cerevisiae using the lithium acetate or spheroplast methods as described in EXAMPLE 2. Homologous recombination takes place in vivo in the transformed yeast cells. Once the target DNA is captured by the vector via homologous recombination, the DNA can be stably replicated in both yeast and bacterial systems. As indicated in FIG. 1, the gap filling vector contains the selectable marker TRP1. Therefore, the yeast cells are plated on tryptophan-free medium to screen for positive transformants. Transformed strains growing on the tryptophan-free medium are screened for the presence of target DNA inserts in the gap filling plasmid. Such screening can be by any known method in the art, for example, restriction enzyme digestion, PCR and/or gel electrophoresis.

Yeast transformants screened by PCR using primers specific for chloroplast genome DNA (SEQ IDs 89 and 90). Desired clones are those that give a PCR product of expected size. DNA is prepared from the isolated yeast clones and transformed into bacteria. Bacterial transformants are PCR screened using primers specific for chloroplast genome DNA (SEQ IDs 89 and 90). Desired clones are those that give a PCR product of expected size. DNA is prepared from the isolated bacterial clones and analyzed by restriction digest with EcoRI.

### Example 12

### Reassembly on a Complete Chloroplast Genome

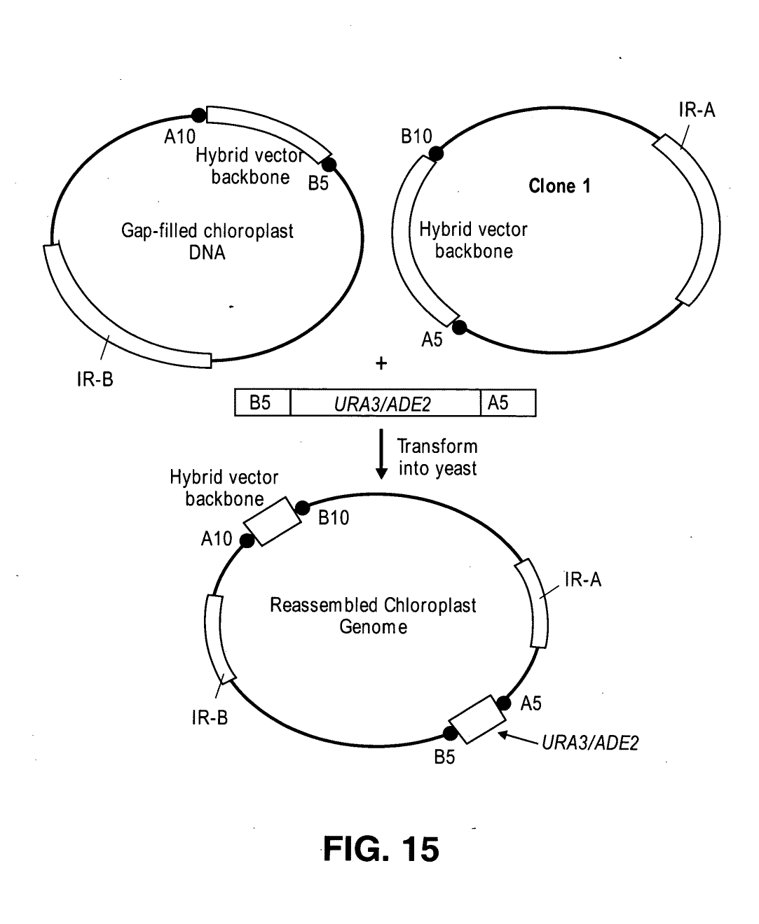

In some instances, the chloroplast genome may be divided into different plasmids, which, in total, comprise the entirety of the genome (FIG. 15). In such instances, capture of smaller portions may facilitate rapid and complex modification of multiple positions within the genome. The chloroplast genome fragments are then combined to reform a complete (and possibly modified) chloroplast genome.

In this example, the chloroplast is divided between two vectors. One vector consists of the chloroplast genome DNA from B5 to A10, or nucleotide 76,400 to 176,500 (according to the sequence available from NCBI, NC_{—}005353), respectively. This vector may be obtained by the procedure describe in EXAMPLE 10. The other vector consists of the chloroplast genome DNA from B10 to A5, or nucleotide 176,500 to 76,400 (according to the sequence available from NCBI, NC_{—}005353), respectively. This vector is the same one as Clone 1 described in may be obtained by the procedure described in EXAMPLE 6. Both vector share the hybrid vector sequence and can use that as a region of homology for recombination. In this example, the recombination event inserts the hybrid vector sequence between regions A10 and B10 (FIG. 5). However, there is no homology sufficient to facilitate recombination that joins regions A5 and B5 (FIG. 5). Thus, a third vector is added that has selectable markers between sequences homologous to A5 and B5.

The chloroplast genome is reassembled by combining all three vectors and transforming S. cerevisiae using the lithium acetate or spheroplast methods as described in EXAMPLE 2. Homologous recombination takes place in vivo in the transformed yeast cells. Once the target DNA is created via homologous recombination, the DNA can be stably replicated in both yeast and bacterial systems. As indicated in FIG. 15, the third vector contains the selectable markers URA3 and ADE2. Therefore, the yeast cells are plated on tryptophan-free medium to screen for positive transformants. Transformed strains growing on the uracil and/or adenine-free medium are screened for the presence of target DNA inserts in the gap filling plasmid. Such screening can be by any known method in the art, for example, restriction enzyme digestion, PCR and/or gel electrophoresis.

Yeast transformants screened by PCR using primers specific for chloroplast genome DNA (SEQ IDs 89 and 90). Desired clones are those that give a PCR product of expected size. DNA is prepared from the isolated yeast clones and transformed into bacteria. Bacterial transformants are PCR screened using primers specific for chloroplast genome DNA (SEQ IDs 89 and 90). Desired clones are those that give a PCR product of expected size. DNA is prepared from the isolated bacterial clones and analyzed by restriction digest with EcoRI.

### Example 13

### Vectors and Methods to Remove Regions Chloroplast Genome DNA in an Exogenous Host

To generate vectors that remove regions of chloroplast genome DNA, 800-1000 bp regions of chloroplast DNA are amplified using PCR primer pairs that anneal to 5′ and 3′ regions flanking the sites indicated in FIG. 4 (site 1-5′, SEQ ID NOs. 29 and 30; site 1-3′, SEQ ID NOs. 31 and 32; site 3-5′, SEQ ID NOs. 33 and 34; site 3-3′, SEQ ID NOs. 35 and 36; site 4-5′, SEQ ID NOs. 37 and 38; site 4-3′, SEQ ID NOs. 39 and 40; site 5-5′, SEQ ID NOs. 41 and 42; site 5-3′, SEQ ID NOs. 43 and 44; and site 7-5′, SEQ ID NOs. 45 and 46; site 7-3′, SEQ ID NOs. 47 and 48). Pairs of PCR products from the 5′ and 3′ regions for different sites are digested with NotI and I-SceI, mixed, and ligated to NotI-digested pUC-SE (SEQ ID NO. 2). Pairs of yeast selection markers (described in EXAMPLE 4) are cloned between the 5′ and 3′ fragments using Sail.

Regions of chloroplast genome DNA are removed by transforming the deletion vector into yeast that harbor the captured DNA. For transformation, all modification vectors were linearized by digestion with NotI. Yeast harboring the desired clone are grown to saturation in CSM-Trp media at 30° C., then diluted 1:20 in YPAD and grown for 4 hours at 30° C. Yeast were transformed using the lithium-acetate method and transformants were selected for by growth on CSM-Ura media. Transformants were propagated on CSM-Trp-Ura media and then PCR screened using primers specific for the targeted region and a region of the chloroplast genome DNA not targeted by the deletion vector. Desired clones are those that give not product for the targeted region, but do give a PCR product of expected size for the untargeted area. DNA is prepared from the isolated yeast clones and transformed into bacteria. Bacteria PCR screened using primers specific for the targeted region and a region of the chloroplast genome DNA not targeted by the deletion vector. Desired clones are those that give not product for the targeted region, but do give a PCR product of expected size for the untargeted area. DNA is prepared from the isolated bacterial clones and analyzed by restriction digest.

### Example 14

### Reassembly on a Complete Chloroplast Genome

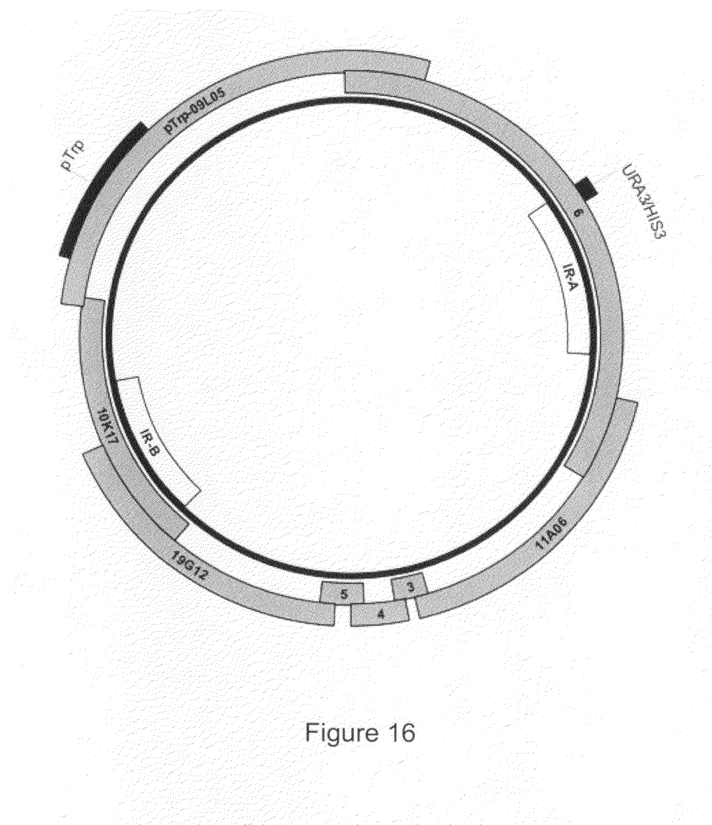

In some instances, the chloroplast genome may be divided into different plasmids, which, in total, comprise the entirety of the genome (FIG. 16). In such instances, capture of smaller portions may facilitate rapid and complex modification of multiple positions within the genome. The chloroplast genome fragments are then combined to reform a complete (and possibly modified) chloroplast genome.

In this example, the chloroplast genome is divided between eight plasmids. One plasmid is Clone 1 from EXAMPLE 6. Four of the plasmids are derived from clones in the C. reinhardtii bacterial artificial chromosome (BAC) library (available from the Clemson University Genomics Institute). The clones are 09L05, 10K17, 11A06, and 19G12, which correspond to chloroplast genome DNA from nucleotide 170,760 to 009,845 (wrapping through nucleotide 000,001), 135,513 to 171,996, 061,923 to 101,692, and 112,250 to 151,666, respectively (according to the sequence available from NCBI, NC_{—}005353). The remaining 3 plasmids were derived from plasmids in the Chlamydomonas Resource Center collection (hosted at Duke University). Clone 3 was generated by PCR amplifying chloroplast genome DNA fragments using primers specific for a 3.0 kb region in P-78 (SEQ ID NOs. 100 and 101) and a 1.7 kb region in P-585 (SEQ ID NOs. 102 and 103). These amplified fragments were mixed and PCR amplified to assemble a 4.7 kb fragment using primers specific for the 5′ end of the product derived from P-78 and the 3′ end of the product derived from P-585 (SEQ ID NOs. 104 and 105). The resulting product was digested with NotI and ligated to NotI-digested pUC-SE (SEQ ID NO. 2), producing Clone 3. The DNA sequence between the NotI restriction sites in Clone 3 correspond to chloroplast genome DNA from nucleotide 099,783 to 104,383 (according to the sequence available from NCBI, NC_{—}005353). Clone 5 was generated by PCR amplifying chloroplast genome DNA fragments using primers specific for a 1.9 kb region in P-586 (SEQ ID NOs. 106 and 107) and a 4.0 kb region in P-19 (SEQ ID NOs. 108 and 109). These amplified fragments were mixed and PCR amplified to assemble a 5.9 kb fragment using primers specific for the 5′ end of the product derived from P-586 and the 3′ end of the product derived from P-19 (SEQ ID NOs. 110 and 111). The resulting product was digested with NotI and ligated to NotI-digested pUC-SE (SEQ ID NO. 2), producing Clone 5. The DNA sequence between the NotI restriction sites in Clone 5 correspond to chloroplast genome DNA from nucleotide 108,427 to 114,337 (according to the sequence available from NCBI, NC_{—}005353). Clone 4 was obtained from S. Mayfield (The Scripps Research Institute, La Jolla, Calif.) and contains a DNA fragment that corresponds to chloroplast genome DNA from nucleotide 102,957 to 110,192 (according to the sequence available from NCBI, NC_{—}005353). The chloroplast genome DNA sequence in Clone 4 is flanked by NotI and XhoI restriction sites.

Two of the plasmids were modified to contain yeast elements, thereby reducing the number of DNA fragments required for efficient chloroplast genome assembly. First, the URA3 and HIS3 genes were introduced into the region adjacent to chloroplast genome nucleotide 034,410 (according to the sequence available from NCBI, NC_{—}005353) in Clone 1, creating Clone 6. Briefly, yeast harboring Clone 1 (from EXAMPLE 6) were grown to saturation in CSM-Trp media at 30° C., then diluted 1:20 in YPAD and grown for 4 hours at 30° C. Yeast were transformed with the NotI fragment from pUC3-URA3/HIS3 plasmid (described in EXAMPLE 4) using the lithium-acetate method and transformants were by growth on CSM-Ura-His agar medium. Transformants were propagated on CSM-Trp-Ura-His media and then PCR screened using primers specific for proper integration of the URA3/HIS3 cassette (SEQ ID NOs. 112 and 113 and SEQ ID NOs. 114 and 115). Desired clones are those that give PCR products of expected size for both reactions. DNA was prepared from the isolated yeast clones and transformed into bacteria. Bacteria were PCR screened using primers specific for proper integration of the URA3/HIS3 cassette (SEQ ID NOs. 112 and 113 and SEQ ID NOs. 114 and 115). Desired clones are those that give PCR products of expected size for both reactions. DNA was prepared from the isolated bacterial clones and used for chloroplast genome assembly. Second, the hybrid vector elements were inserted into the region adjacent to chloroplast genome nucleotide 176,500 (according to the sequence available from NCBI, NC_{—}005353) in 09L05, creating pTrp-09L05. Briefly, pTrp-10 was generated in the same fashion as pTrp-10-Kan (described in EXAMPLE 3), except that pDOCI-10 (SEQ ID NO. 3) was used instead of pDOCI-10-Kan. pTRP-10 that was digested with NotI to linearize the vector such that the targeting elements on are on each end (FIG. 5). Linearized pTrp-10 was mixed with the circular 09L05 plasmid and transformed into yeast using the lithium acetate method. Transformants were selected for by growth on CSM-Trp agar medium. Transformants were propagated on CSM-Trp-media and then PCR screened using primers specific for proper integration of the pTrp-10 (SEQ ID NOs. 116 and 117 and SEQ ID NOs. 118 and 119). Desired clones are those that give PCR products of expected size for both reactions. DNA was prepared from the isolated yeast clones and transformed into bacteria. Bacteria were PCR screened using primers specific for proper integration of pTrp-10 (SEQ ID NOs. 116 and 117 and SEQ ID NOs. 118 and 119). Desired clones are those that give PCR products of expected size for both reactions. DNA was prepared from the isolated bacterial clones and used for chloroplast genome assembly.

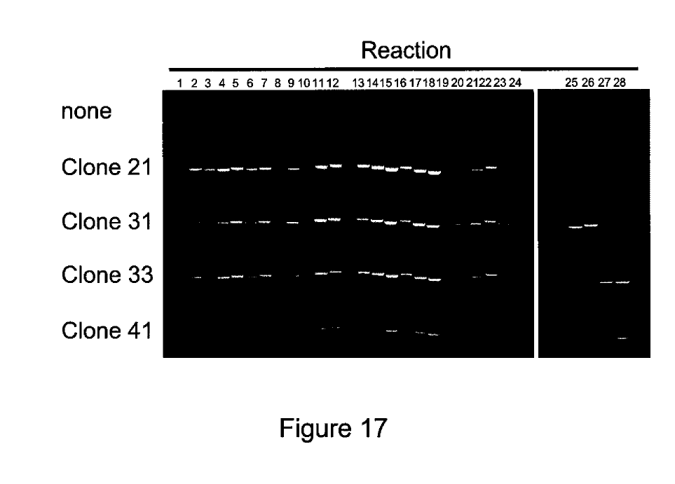

Each of the eight plasmids was digested to liberate the chloroplast genome DNA. pTrp-09L05, 10K17, 11A06, 19G12, and Clone 3 and Clone 5 were digested with NotI, which cuts twice in each plasmid; Clone 4 was digested with NotI and XhoI, and Clone 6 was digested with RsrII and PasI. To assemble the chloroplast genome by homologous recombination in yeast, the chloroplast genome DNA fragments were mixed in approximately equimolar ratios and transformed into yeast cells using the spheroplast method (described in EXAMPLE 2). Transformants were selected by growth on CSM-Ura-His agar medium, propagated on CSM-Trp-Ura-His media, and PCR screened using primers that amplify 24 different regions spread throughout the entire chloroplast genome (SEQ ID NOs. 122 and 123; 124 and 125; 126 and 127; 112 and 113; 114 and 115; 128 and 129; 130 and 131; 132 and 133; 134 and 135; 136 and 137; 138 and 139; 140 and 141; 142 and 143; 144 and 145; 146 and 147; 148 and 149; 150 and 151; 152 and 153; 154 and 155; 156 and 157; 116 and 117; 118 and 119; 158 and 159; and 160 and 161). FIG. 17 shows that PCR products are observed for all 24 reactions when Clone 21 is used as a template, but not when DNA prepared from wild type yeast (negative control).

Once assembled, clone 21 can be transformed into a suitable bacterial host such as E. coli for amplification using conventional methods. Briefly, the transformed bacterial host cells as identified by PCR are grown to near saturable levels of growth, the cells collected and lysed, and the amplified clone isolated. Various techniques for the isolation of non-chomrosomal DNA from bacteria are known in the art.

### Example 15

### One-Step PCR-Mediated Stabilization and/or Modification of Large DNA Molecules

Often, selection markers are inserted into heterologous DNA to stabilize fragile regions and/or modify target regions (described in EXAMPLE 4). As new target sites are identified, new vectors are required to mediate efficient insertion of the selection marker(s) into the target region. While these vectors are easily generated, cloning the DNA sequences requires multiple steps, thereby increasing the time required to insert the selection marker(s). A more rapid method of targeting stabilization elements is therefore desirable.

In this example, we describe a one-step PCR-mediated technique for introducing selection markers into target regions. Briefly, two PCR primers are designed such that the sequence of the first 40-42 nucleotides (5′->3′) of each primer are identical to the target sequences, and the final 18-20 nucleotides are identical to sequences within a vector containing a selection marker cassette. Thus, PCR amplification of the selection marker cassette adds flanking sequences that target the selection marker(s) to the desired region.

Define target at Clone 31. To introduce a selection marker cassette consisting of the yeast LYS2 and bacterial gentamycin resistance (Gent^{r}) genes into the region adjacent to chloroplast genome nucleotide 129,578 (according to the sequence available from NCBI, NC_{—}005353) in Clone 21 (described in EXAMPLE 14), PCR primers (SEQ ID NOs. 120 and 121) were used to amplify the 5.8 kbp Gent^{r}(LYS2 region from pUC7-URA3/Gent^{r}/LYS2 (described in EXAMPLE 4). The PCR product was then transformed in yeast harboring Clone 21 by the lithium acetate method (described in EXAMPLE 2). Transformants were selected by growth on CSM-Ura-His-Lys agar medium, propagated in CSM-Trp-Ura-His-Lys media, and PCR screened using primers that amplify 24 different regions spread throughout the entire chloroplast genome (SEQ ID NOs. 122 and 123; 124 and 125; 126 and 127; 112 and 113; 114 and 115; 128 and 129; 130 and 131; 132 and 133; 134 and 135; 136 and 137; 138 and 139; 140 and 141; 142 and 143; 144 and 145; 146 and 147; 148 and 149; 150 and 151; 152 and 153; 154 and 155; 156 and 157; 116 and 117; 118 and 119; 158 and 159; and 160 and 161). FIG. 17 shows that PCR products are observed for all 24 PCR reactions when Clone 21 is used as a template, but not when DNA prepared from wild type yeast. Clone 31 was also PCR screened using primers specific for proper integration of the Gent^{r}/LYS2 cassette (SEQ ID NOs. 162 and 163 and SEQ ID NOs. 164 and 165). FIG. 17 shows that PCR products are observed for both PCR reactions wherein Clone 31 is used as a template, but not when DNA prepared from wild type yeast or Clone 21.

Define target at Clone 33. To introduce a selection marker cassette consisting of the yeast LEU2 and bacterial chloramphenicol acetyltransferase (CAT) genes into the region adjacent to chloroplast genome nucleotide 057,987 (according to the sequence available from NCBI, NC_{—}005353) in Clone 21 (described in EXAMPLE 14), PCR primers (SEQ ID NO. 166 and 167) were used to amplify the 3.4 kbp CAT/LEU2 region from pUC3-URA3/CAT/LEU2 (described in EXAMPLE 4). The PCR product was then transformed in yeast harboring Clone 21 by the lithium acetate method (described in EXAMPLE 2). Transformants were selected by growth on CSM-Ura-His-Leu agar medium, propagated in CSM-Trp-Ura-His-Leu media, and PCR screened using primers that amplify 24 different regions spread throughout the entire chloroplast genome (SEQ ID NOs. 122 and 123; 124 and 125; 126 and 127; 112 and 113; 114 and 115; 128 and 129; 130 and 131; 132 and 133; 134 and 135; 136 and 137; 138 and 139; 140 and 141; 142 and 143; 144 and 145; 146 and 147; 148 and 149; 150 and 151; 152 and 153; 154 and 155; 156 and 157; 116 and 117; 118 and 119; 158 and 159; and 160 and 161). FIG. 17 shows that PCR products are observed for all 24 PCR reactions when Clone 33 is used as a template, but not when DNA prepared from wild type yeast. Clone 33 was also PCR screened using primers specific for proper integration of the CAT/LEU2 cassette (SEQ ID NOs. 168 and 169 and SEQ ID NOs. 170 and 171). FIG. 17 shows that PCR products are observed for both PCR reactions wherein Clone 33 is used as a template, but not when DNA prepared from wild type yeast or Clone 21.

Define target at Clone 41. To introduce a selection marker cassette consisting of the yeast LEU2 and bacterial chloramphenicol acetyltransferase (CAT) genes into the region adjacent to chloroplast genome nucleotide 057,987 (according to the sequence available from NCBI, NC_{—}005353) in Clone 21 (described in EXAMPLE 14), PCR primers (SEQ ID NO. 166 and 167) were used to amplify the 3.4 kbp CAT/LEU2 region from pUC3-URA3/CAT/LEU2 (described in EXAMPLE 4). The PCR product was then transformed in yeast harboring Clone 21 by the lithium acetate method (described in EXAMPLE 2). Transformants were selected by growth on CSM-Ura-His-Leu agar medium, propagated in CSM-Trp-Ura-His-Leu media, and PCR screened using primers that amplify 24 different regions spread throughout the entire chloroplast genome (SEQ ID NOs. 122 and 123; 124 and 125; 126 and 127; 112 and 113; 114 and 115; 128 and 129; 130 and 131; 132 and 133; 134 and 135; 136 and 137; 138 and 139; 140 and 141; 142 and 143; 144 and 145; 146 and 147; 148 and 149; 150 and 151; 152 and 153; 154 and 155; 156 and 157; 116 and 117; 118 and 119; 158 and 159; and 160 and 161). FIG. 17 shows that PCR products are observed for all 24 PCR reactions wherein Clone 41 is used as a template, but not when DNA prepared from wild type yeast. Clone 41 was also PCR screened using primers specific for proper integration of the Gent^{r}/LYS2 cassette (SEQ ID NOs. 162 and 163 and SEQ ID NOs. 164 and 165) and the CAT/LEU2 cassette (SEQ ID NOs. 168 and 169 and SEQ ID NOs. 170 and 171). FIG. 17 shows that PCR products are observed for all four PCR reactions wherein Clone 41 is used as a template, but not when DNA prepared from wild type yeast or Clone 21, Clone 31, or Clone 33.

FIG. 17 description. The template for each reaction was DNA prepared from yeast containing the indicated clone. The indicated reaction columns refer to the primer pairs used: 1) SEQ ID NOs. 122 and 123; 2) SEQ ID NOs. 124 and 125; 3) SEQ ID NOs. 126 and 127; 4) SEQ ID NOs. 112 and 113; 5) SEQ ID NOs. 114 and 115; 6) SEQ ID NOs. 128 and 129; 7) SEQ ID NOs. 130 and 131; 8) SEQ ID NOs. 132 and 133; 9) SEQ ID NOs. 134 and 135; 10) SEQ ID NOs. 136 and 137; 11) SEQ ID NOs. 138 and 139; 12) SEQ ID NOs. 140 and 141; 13) SEQ ID NOs. 142 and 143; 14) SEQ ID NOs. 144 and 145; 15) SEQ ID NOs. 146 and 147; 16) SEQ ID NOs. 148 and 149; 17) SEQ ID NOs. 150 and 151; 18) SEQ ID NOs. 152 and 153; 19) SEQ ID NOs. 154 and 155; 20) SEQ ID NOs. 156 and 157; 21) SEQ ID NOs. 116 and 117; 22) SEQ ID NOs. 118 and 119; 23) SEQ ID NOs. 158 and 159; 24) SEQ ID NOs. 160 and 161; 25) SEQ ID NOs. 162 and 163; 26) SEQ ID NOs. 164 and 165; 27) SEQ ID NOs. 168 and 169; and 28) SEQ ID NOs. 170 and 171.

Various modifications, processes, as well as numerous structures that may be applicable herein will be apparent. Various aspects, features or embodiments may have been explained or described in relation to understandings, beliefs, theories, underlying assumptions, and/or working or prophetic examples, although it will be understood that any particular understanding, belief, theory, underlying assumption, and/or working or prophetic example is not limiting. Although the various aspects and features may have been described with respect to various embodiments and specific examples herein, it will be understood that any of same is not limiting with respect to the full scope of the appended claims or other claims that may be associated with this application.

What is claimed is:

1. A method of producing a vector containing a reconstructed genome, comprising
(a) introducing two or more vectors into a host cell, wherein said vectors comprise fragments of a genome
(b) recombining said vectors into a single vector comprising at least about 90% of a genome, thereby producing a reconstructed genome.

2. The method of claim 1, wherein said reconstructed genome has at least one modification.

3. The method of claim 2, wherein said modification is made by homologous recombination in a yeast cell using a targeted integration vector.

4. The method of claim 3, wherein said targeted integration vector comprises a segment containing the modification to be introduced and two targeting segments of 40 to 1000 bases on each end of the integration vector.

5. The method of claim 4, wherein the targeted integration vector is produced using the polymerase chain reaction and said targeting segments are from about 40 bases to about 200 bases.

6. The method of claim 5, wherein said targeted integration vector is made using a set of primers, each primer comprising an area of 18 to 20 bases identical to the segment of modified DNA and an area of about 40 to 200 bases identical to the targeted integration site.

7. The method of claim 2, wherein said at least one modification comprises an addition, deletion, mutation or rearrangement.

8. The method of claim 1, wherein said fragments of the genome are obtained from a non vascular photosynthetic organism.

9. The method of claim 8, wherein said photosynthetic organism is a microalgae or a macroalgae.

10. The method of claim 8, wherein said photosynthetic organism is selected from the group consisting of Ch. Vulgaris, C. reinhardtii, D. salina, S. quadricanda and H. pluvalis.

11. The method of claim 2, wherein said at least one modification introduces an exogenous coding region.

12. The method of claim 11, wherein said exogenous coding region encodes an enzyme in a pathway for synthesis of a fatty acid, a terpene or a terpenoid.

13. A plant comprising a reconstituted genome of claim 1.

14. The plant of claim 13, wherein said plant is a non-vascular photosynthetic organism.

15. The plant of claim 14, wherein the non-vascular photosynthetic organisms is a microalgae or a macroalgae.

16. The plant of claim 14, wherein the non-vascular photosynthetic organisms is selected from the group consisting of Ch. vulgaris, C. reinhardtii D. salina, S. quadricanda and H. pluvalis.

17. The plant of claim 13, wherein said plant is non-photosynthetic.

18. The plant of claim 17, wherein said plant is non-photosynthetic due to a natural or induced mutation.

19. The plant of claim 17, wherein said plant is made photoautotrophic by the presence of the synthetic chloroplast genome.

20. A method for stabilizing and or modifying one or more target regions of DNA comprising:
obtaining a DNA cassette comprising a selection marker, a heterologous DNA sequence or both;
obtaining a set of primers, said primers comprising in a 5′ to 3′ order, approximately 40-200 nucleotides identical to a sequence in a target region to be modified and 18 to 20 nucleotides identical to a 3′ end sequence of the DNA cassette;
amplifying the DNA cassette by polymerase chain reaction to produce an insertion vector comprising the DNA cassette flanked by nucleotide sequences identical to the target region to be modified or stabilized; and
inserting said insertion vector into the target region of DNA by homologous recombination.

21. The method of claim 20, wherein said primers comprise, in a 5′ to 3′ order, approximately 40-60 nucleotides identical to a sequence in the target region to be modified and 18 to 20 nucleotides identical to a 3′ end sequence of the DNA cassette.
